SEQUENCE LISTING (119 sequences)

SEQ ID NO: 1 - DNA, 2829 bp, Homo sapiens
ccgccccactgcagtcgagaagctgtggggaggggcggcgtcggaggaagccgccagcccttatggggccagctccaagcccgtttccaccgcggcattggtcaggctgggccggacgaacgaggcggcgtcggcggtgcggggggtggtgggtgggtccccggctcgctgggggcggagcgcgggccggtccacctggcgggctccccggcgatgagcgcgccggccgctcgctcggcttccggggctgaggctgcggggggaaggtggggaaccaaacgcgcgtcaacgcgggcgcgggcccggggcagaccccgcccgggccggccctgcccgcacctcccccaagcgaagtcggcagtttcgtttgctcggttggttttggagtcttgagtccgtgggtgccgcgactcggtctgagacacggcgggggcggggcgggcgctcggagccgcggtgagtcagggctccgcgcccgccgactcatttctgccgccccggcccgggagcgcgatttgcaatgcaaagtcaccccgcctccagcaccccaatctgccccaggatccgccagcactagagacctcaacggcccgacggccgctcccctcccctcgtctacccctccctcgtcggcggctgagccgcgaggggaagttttgcaatcccggacaaacaaacgccggtcttgcacgggcttgaaaaactttgggggaaatgaagagtgagcgaaatcgaagccatcgctcgggcctggcgctcggctccgcgggctcctgggggcgcgacccgccgggcctgcccaccccgtccctccaccccggcccccggccctccctcctccctgcctcccggctgttacctcataggtcgagggcgctcagtagccccctaaccagctggagaagtcgagtagctcgcgctccgcaggactcagcgcgccttcgcagccgctgtcgtccgacgagtaggcggaacgcggggagccgggctccgagctgcccccgcggcccggggacgaagaagcgcgggagggcgaggcggcgaccggggtggtccctggcggcccgcggggcgcagacggccgcacggcctgcggcctcagccctcccgccagcgcgttgcgcacggcgtcgtgctcggccagcaggcgctgcagcgcgcggatgtactccacggctgagcgcagcgtctccaccttgctcagcttcttgctggcgccgccgtgcggcacgtgctgccgcagcgcctggaagcccaagttcaccagcttcacgcggttgcgctcgcgctcattgcgccgcgctacggccgctgcgccgcctccggtctctgcggtggccggtcgccgccgccggctgcagcgcaacagttccggggacgcgggtctccgccgggcagcgcagccgacagggacggggggcgcagggggcgcggacctgggcagtgtgccgccgtccatcgcgcctgcatccacccgcccgctccaggtcccggcgcgccgcaggaaggtgcaggcagaggaaccggaggcgacggggaaaactgtggcgccccaagggggcttctggcacggcgccgccaggcaactccccagggcacgcgtcctaggtcgtctggagcccggggataggaggcctagtggtggcaggccgtacgcgccagggagcgtgggacgctcgtgtcccgcgcgtgcggccggactctcccaggtctccgcaggcgcggcgcaggcggctggtttttaaatgtatagataaccctcctccgcgccgccgccgtcgcctttctcacgccctccttccttcgcctcgccctcccgccacgcttcgccctccccctcgcgcgatcacattctgtaaggcccaaagcgtgcgcatgtccccctagcccatcccccggacgcagtccacagatccccagtgcgcccaactggcgaaatctgcgagttcccggtgcgccccctgctcccggcaggtgcttagtgcgcccccaaagcaaggtacgcaggtcctgggttgagccttcccgtacccccaccctaaccccgcgcgcagccccgccagtcccaagagccgccagaccttcgcacgcgcagcgcgcgctgtgggagggaaggcgcggccctggcgacaacacggctgttcgggaggcgcgcaagatccccgggggcagcacgcgccgcgcagcccacacccacgccccaccctcctggggccgaggaggcgggggccagggtctcagccaatcgtgggccacccgtttggccaatcgcgcagggcgcggctccacgcccggccccattgaggaagcgcgtacgcgtggcgcgtggctcacggggagcatcgctaacaaagctgggttcctgctgggccccgccctgctcctcgcccccgcgactgggctgggcgcgctgtcccctagcgcagctatgtcccgagcgcgcccccacctgtgcgttaatctactgggaatgggggtggactgcgccttacctggggcggggtggggcttaaggagtggtcgagactgaggcggggtgggaggttcaggttcccggggcgccttccccaacccgccccgctttccccgtccctccacgcgcaccctgcctgtggtttccgtgcgcccccggcctgagggctctgggcggcaccttaacccggagggcctggaggtctgcacccgaccgccttgtgccaggacggtcaggtccacgccctcccccaccgtggctccctccatctgcagtatcccccacctccagcccgtcctgccctcctgttctccgtctcgcttcccgtcggtgcctccgggatctcacagccctcg

SEQ ID NO: 2 - DNA, 2829 bp, Artificial Sequence
  converted
tcgttttattgtagtcgagaagttgtggggaggggcggcgtcggaggaagtcgttagtttttatggggttagttttaagttcgtttttatcgcggtattggttaggttgggtcggacgaacgaggcggcgtcggcggtgcggggggtggtgggtgggttttcggttcgttgggggcggagcgcgggtcggtttatttggcgggtttttcggcgatgagcgcgtcggtcgttcgttcggttttcggggttgaggttgcggggggaaggtggggaattaaacgcgcgttaacgcgggcgcgggttcggggtagatttcgttcgggtcggttttgttcgtattttttttaagcgaagtcggtagtttcgtttgttcggttggttttggagttttgagttcgtgggtgtcgcgattcggtttgagatacggcgggggcggggcgggcgttcggagtcgcggtgagttagggtttcgcgttcgtcgatttatttttgtcgtttcggttcgggagcgcgatttgtaatgtaaagttatttcgtttttagtattttaatttgttttaggattcgttagtattagagattttaacggttcgacggtcgttttttttttttcgtttatttttttttcgtcggcggttgagtcgcgaggggaagttttgtaatttcggataaataaacgtcggttttgtacgggtttgaaaaattttgggggaaatgaagagtgagcgaaatcgaagttatcgttcgggtttggcgttcggtttcgcgggtttttgggggcgcgattcgtcgggtttgtttatttcgtttttttatttcggttttcggtttttttttttttttgtttttcggttgttattttataggtcgagggcgtttagtagttttttaattagttggagaagtcgagtagttcgcgtttcgtaggatttagcgcgttttcgtagtcgttgtcgttcgacgagtaggcggaacgcggggagtcgggtttcgagttgttttcgcggttcggggacgaagaagcgcgggagggcgaggcggcgatcggggtggtttttggcggttcgcggggcgtagacggtcgtacggtttgcggttttagttttttcgttagcgcgttgcgtacggcgtcgtgttcggttagtaggcgttgtagcgcgcggatgtattttacggttgagcgtagcgtttttattttgtttagttttttgttggcgtcgtcgtgcggtacgtgttgtcgtagcgtttggaagtttaagtttattagttttacgcggttgcgttcgcgtttattgcgtcgcgttacggtcgttgcgtcgttttcggtttttgcggtggtcggtcgtcgtcgtcggttgtagcgtaatagtttcggggacgcgggttttcgtcgggtagcgtagtcgatagggacggggggcgtagggggcgcggatttgggtagtgtgtcgtcgtttatcgcgtttgtatttattcgttcgttttaggtttcggcgcgtcgtaggaaggtgtaggtagaggaatcggaggcgacggggaaaattgtggcgttttaagggggtttttggtacggcgtcgttaggtaattttttagggtacgcgttttaggtcgtttggagttcggggataggaggtttagtggtggtaggtcgtacgcgttagggagcgtgggacgttcgtgtttcgcgcgtgcggtcggatttttttaggttttcgtaggcgcggcgtaggcggttggtttttaaatgtatagataattttttttcgcgtcgtcgtcgtcgttttttttacgttttttttttttcgtttcgttttttcgttacgtttcgttttttttttcgcgcgattatattttgtaaggtttaaagcgtgcgtatgtttttttagtttatttttcggacgtagtttatagatttttagtgcgtttaattggcgaaatttgcgagttttcggtgcgttttttgttttcggtaggtgtttagtgcgtttttaaagtaaggtacgtaggttttgggttgagttttttcgtatttttattttaatttcgcgcgtagtttcgttagttttaagagtcgttagattttcgtacgcgtagcgcgcgttgtgggagggaaggcgcggttttggcgataatacggttgttcgggaggcgcgtaagattttcgggggtagtacgcgtcgcgtagtttatatttacgttttatttttttggggtcgaggaggcgggggttagggttttagttaatcgtgggttattcgtttggttaatcgcgtagggcgcggttttacgttcggttttattgaggaagcgcgtacgcgtggcgcgtggtttacggggagtatcgttaataaagttgggtttttgttgggtttcgttttgtttttcgttttcgcgattgggttgggcgcgttgttttttagcgtagttatgtttcgagcgcgtttttatttgtgcgttaatttattgggaatgggggtggattgcgttttatttggggcggggtggggtttaaggagtggtcgagattgaggcggggtgggaggtttaggttttcggggcgttttttttaattcgtttcgtttttttcgtttttttacgcgtattttgtttgtggttttcgtgcgttttcggtttgagggttttgggcggtattttaattcggagggtttggaggtttgtattcgatcgttttgtgttaggacggttaggtttacgtttttttttatcgtggttttttttatttgtagtattttttatttttagttcgttttgtttttttgtttttcgtttcgtttttcgtcggtgttttcgggattttatagttttcg

SEQ ID NO: 3 - DNA, 2829 bp, Artificial Sequence
  converted
cgaaaactataaaatcccgaaaacaccgacgaaaaacgaaacgaaaaacaaaaaaacaaaacgaactaaaaataaaaaatactacaaataaaaaaaaccacgataaaaaaaaacgtaaacctaaccgtcctaacacaaaacgatcgaatacaaacctccaaaccctccgaattaaaataccgcccaaaaccctcaaaccgaaaacgcacgaaaaccacaaacaaaatacgcgtaaaaaaacgaaaaaaacgaaacgaattaaaaaaaacgccccgaaaacctaaacctcccaccccgcctcaatctcgaccactccttaaaccccaccccgccccaaataaaacgcaatccacccccattcccaataaattaacgcacaaataaaaacgcgctcgaaacataactacgctaaaaaacaacgcgcccaacccaatcgcgaaaacgaaaaacaaaacgaaacccaacaaaaacccaactttattaacgatactccccgtaaaccacgcgccacgcgtacgcgcttcctcaataaaaccgaacgtaaaaccgcgccctacgcgattaaccaaacgaataacccacgattaactaaaaccctaacccccgcctcctcgaccccaaaaaaataaaacgtaaatataaactacgcgacgcgtactacccccgaaaatcttacgcgcctcccgaacaaccgtattatcgccaaaaccgcgccttccctcccacaacgcgcgctacgcgtacgaaaatctaacgactcttaaaactaacgaaactacgcgcgaaattaaaataaaaatacgaaaaaactcaacccaaaacctacgtaccttactttaaaaacgcactaaacacctaccgaaaacaaaaaacgcaccgaaaactcgcaaatttcgccaattaaacgcactaaaaatctataaactacgtccgaaaaataaactaaaaaaacatacgcacgctttaaaccttacaaaatataatcgcgcgaaaaaaaaaacgaaacgtaacgaaaaaacgaaacgaaaaaaaaaaaacgtaaaaaaaacgacgacgacgacgcgaaaaaaaattatctatacatttaaaaaccaaccgcctacgccgcgcctacgaaaacctaaaaaaatccgaccgcacgcgcgaaacacgaacgtcccacgctccctaacgcgtacgacctaccaccactaaacctcctatccccgaactccaaacgacctaaaacgcgtaccctaaaaaattacctaacgacgccgtaccaaaaacccccttaaaacgccacaattttccccgtcgcctccgattcctctacctacaccttcctacgacgcgccgaaacctaaaacgaacgaataaatacaaacgcgataaacgacgacacactacccaaatccgcgccccctacgccccccgtccctatcgactacgctacccgacgaaaacccgcgtccccgaaactattacgctacaaccgacgacgacgaccgaccaccgcaaaaaccgaaaacgacgcaacgaccgtaacgcgacgcaataaacgcgaacgcaaccgcgtaaaactaataaacttaaacttccaaacgctacgacaacacgtaccgcacgacgacgccaacaaaaaactaaacaaaataaaaacgctacgctcaaccgtaaaatacatccgcgcgctacaacgcctactaaccgaacacgacgccgtacgcaacgcgctaacgaaaaaactaaaaccgcaaaccgtacgaccgtctacgccccgcgaaccgccaaaaaccaccccgatcgccgcctcgccctcccgcgcttcttcgtccccgaaccgcgaaaacaactcgaaacccgactccccgcgttccgcctactcgtcgaacgacaacgactacgaaaacgcgctaaatcctacgaaacgcgaactactcgacttctccaactaattaaaaaactactaaacgccctcgacctataaaataacaaccgaaaaacaaaaaaaaaaaaaaaccgaaaaccgaaataaaaaaacgaaataaacaaacccgacgaatcgcgcccccaaaaacccgcgaaaccgaacgccaaacccgaacgataacttcgatttcgctcactcttcatttcccccaaaatttttcaaacccgtacaaaaccgacgtttatttatccgaaattacaaaacttcccctcgcgactcaaccgccgacgaaaaaaaaataaacgaaaaaaaaaaaacgaccgtcgaaccgttaaaatctctaatactaacgaatcctaaaacaaattaaaatactaaaaacgaaataactttacattacaaatcgcgctcccgaaccgaaacgacaaaaataaatcgacgaacgcgaaaccctaactcaccgcgactccgaacgcccgccccgcccccgccgtatctcaaaccgaatcgcgacacccacgaactcaaaactccaaaaccaaccgaacaaacgaaactaccgacttcgcttaaaaaaaatacgaacaaaaccgacccgaacgaaatctaccccgaacccgcgcccgcgttaacgcgcgtttaattccccaccttccccccgcaacctcaaccccgaaaaccgaacgaacgaccgacgcgctcatcgccgaaaaacccgccaaataaaccgacccgcgctccgcccccaacgaaccgaaaacccacccaccaccccccgcaccgccgacgccgcctcgttcgtccgacccaacctaaccaataccgcgataaaaacgaacttaaaactaaccccataaaaactaacgacttcctccgacgccgcccctccccacaacttctcgactacaataaaacga

SEQ ID NO: 4 - DNA, 2829 bp, Artificial Sequence
  converted
ccgccccactacaatcgaaaaactataaaaaaaaacgacgtcgaaaaaaaccgccaacccttataaaaccaactccaaacccgtttccaccgcgacattaatcaaactaaaccgaacgaacgaaacgacgtcgacgatacgaaaaataataaataaatccccgactcgctaaaaacgaaacgcgaaccgatccacctaacgaactccccgacgataaacgcgccgaccgctcgctcgacttccgaaactaaaactacgaaaaaaaaataaaaaaccaaacgcgcgtcaacgcgaacgcgaacccgaaacaaaccccgcccgaaccgaccctacccgcacctcccccaaacgaaatcgacaatttcgtttactcgattaattttaaaatcttaaatccgtaaataccgcgactcgatctaaaacacgacgaaaacgaaacgaacgctcgaaaccgcgataaatcaaaactccgcgcccgccgactcatttctaccgccccgacccgaaaacgcgatttacaatacaaaatcaccccgcctccaacaccccaatctaccccaaaatccgccaacactaaaaacctcaacgacccgacgaccgctcccctcccctcgtctacccctccctcgtcgacgactaaaccgcgaaaaaaaattttacaatcccgaacaaacaaacgccgatcttacacgaacttaaaaaactttaaaaaaaataaaaaataaacgaaatcgaaaccatcgctcgaacctaacgctcgactccgcgaactcctaaaaacgcgacccgccgaacctacccaccccgtccctccaccccgacccccgaccctccctcctccctacctcccgactattacctcataaatcgaaaacgctcaataaccccctaaccaactaaaaaaatcgaataactcgcgctccgcaaaactcaacgcgccttcgcaaccgctatcgtccgacgaataaacgaaacgcgaaaaaccgaactccgaactacccccgcgacccgaaaacgaaaaaacgcgaaaaaacgaaacgacgaccgaaataatccctaacgacccgcgaaacgcaaacgaccgcacgacctacgacctcaaccctcccgccaacgcgttacgcacgacgtcgtactcgaccaacaaacgctacaacgcgcgaatatactccacgactaaacgcaacgtctccaccttactcaacttcttactaacgccgccgtacgacacgtactaccgcaacgcctaaaaacccaaattcaccaacttcacgcgattacgctcgcgctcattacgccgcgctacgaccgctacgccgcctccgatctctacgataaccgatcgccgccgccgactacaacgcaacaattccgaaaacgcgaatctccgccgaacaacgcaaccgacaaaaacgaaaaacgcaaaaaacgcgaacctaaacaatataccgccgtccatcgcgcctacatccacccgcccgctccaaatcccgacgcgccgcaaaaaaatacaaacaaaaaaaccgaaaacgacgaaaaaaactataacgccccaaaaaaacttctaacacgacgccgccaaacaactccccaaaacacgcgtcctaaatcgtctaaaacccgaaaataaaaaacctaataataacaaaccgtacgcgccaaaaaacgtaaaacgctcgtatcccgcgcgtacgaccgaactctcccaaatctccgcaaacgcgacgcaaacgactaatttttaaatatataaataaccctcctccgcgccgccgccgtcgcctttctcacgccctccttccttcgcctcgccctcccgccacgcttcgccctccccctcgcgcgatcacattctataaaacccaaaacgtacgcatatccccctaacccatcccccgaacgcaatccacaaatccccaatacgcccaactaacgaaatctacgaattcccgatacgccccctactcccgacaaatacttaatacgcccccaaaacaaaatacgcaaatcctaaattaaaccttcccgtacccccaccctaaccccgcgcgcaaccccgccaatcccaaaaaccgccaaaccttcgcacgcgcaacgcgcgctataaaaaaaaaaacgcgaccctaacgacaacacgactattcgaaaaacgcgcaaaatccccgaaaacaacacgcgccgcgcaacccacacccacgccccaccctcctaaaaccgaaaaaacgaaaaccaaaatctcaaccaatcgtaaaccacccgtttaaccaatcgcgcaaaacgcgactccacgcccgaccccattaaaaaaacgcgtacgcgtaacgcgtaactcacgaaaaacatcgctaacaaaactaaattcctactaaaccccgccctactcctcgcccccgcgactaaactaaacgcgctatcccctaacgcaactatatcccgaacgcgcccccacctatacgttaatctactaaaaataaaaataaactacgccttacctaaaacgaaataaaacttaaaaaataatcgaaactaaaacgaaataaaaaattcaaattcccgaaacgccttccccaacccgccccgctttccccgtccctccacgcgcaccctacctataatttccgtacgcccccgacctaaaaactctaaacgacaccttaacccgaaaaacctaaaaatctacacccgaccgccttataccaaaacgatcaaatccacgccctcccccaccgtaactccctccatctacaatatcccccacctccaacccgtcctaccctcctattctccgtctcgcttcccgtcgatacctccgaaatctcacaaccctcg

SEQ ID NO: 5 - DNA, 2829 bp, Artificial Sequence
  converted
cgagggttgtgagatttcggaggtatcgacgggaagcgagacggagaataggagggtaggacgggttggaggtgggggatattgtagatggagggagttacggtgggggagggcgtggatttgatcgttttggtataaggcggtcgggtgtagatttttaggtttttcgggttaaggtgtcgtttagagtttttaggtcgggggcgtacggaaattataggtagggtgcgcgtggagggacggggaaagcggggcgggttggggaaggcgtttcgggaatttgaattttttatttcgttttagtttcgattattttttaagttttatttcgttttaggtaaggcgtagtttatttttatttttagtagattaacgtataggtgggggcgcgttcgggatatagttgcgttaggggatagcgcgtttagtttagtcgcgggggcgaggagtagggcggggtttagtaggaatttagttttgttagcgatgtttttcgtgagttacgcgttacgcgtacgcgtttttttaatggggtcgggcgtggagtcgcgttttgcgcgattggttaaacgggtggtttacgattggttgagattttggttttcgttttttcggttttaggagggtggggcgtgggtgtgggttgcgcggcgcgtgttgttttcggggattttgcgcgtttttcgaatagtcgtgttgtcgttagggtcgcgtttttttttttatagcgcgcgttgcgcgtgcgaaggtttggcggtttttgggattggcggggttgcgcgcggggttagggtgggggtacgggaaggtttaatttaggatttgcgtattttgttttgggggcgtattaagtatttgtcgggagtagggggcgtatcgggaattcgtagatttcgttagttgggcgtattggggatttgtggattgcgttcgggggatgggttagggggatatgcgtacgttttgggttttatagaatgtgatcgcgcgagggggagggcgaagcgtggcgggagggcgaggcgaaggaaggagggcgtgagaaaggcgacggcggcggcgcggaggagggttatttatatatttaaaaattagtcgtttgcgtcgcgtttgcggagatttgggagagttcggtcgtacgcgcgggatacgagcgttttacgttttttggcgcgtacggtttgttattattaggttttttattttcgggttttagacgatttaggacgcgtgttttggggagttgtttggcggcgtcgtgttagaagtttttttggggcgttatagtttttttcgtcgttttcggtttttttgtttgtatttttttgcggcgcgtcgggatttggagcgggcgggtggatgtaggcgcgatggacggcggtatattgtttaggttcgcgttttttgcgtttttcgtttttgtcggttgcgttgttcggcggagattcgcgttttcggaattgttgcgttgtagtcggcggcggcgatcggttatcgtagagatcggaggcggcgtagcggtcgtagcgcggcgtaatgagcgcgagcgtaatcgcgtgaagttggtgaatttgggtttttaggcgttgcggtagtacgtgtcgtacggcggcgttagtaagaagttgagtaaggtggagacgttgcgtttagtcgtggagtatattcgcgcgttgtagcgtttgttggtcgagtacgacgtcgtgcgtaacgcgttggcgggagggttgaggtcgtaggtcgtgcggtcgtttgcgtttcgcgggtcgttagggattatttcggtcgtcgtttcgttttttcgcgttttttcgttttcgggtcgcgggggtagttcggagttcggtttttcgcgtttcgtttattcgtcggacgatagcggttgcgaaggcgcgttgagttttgcggagcgcgagttattcgatttttttagttggttagggggttattgagcgttttcgatttatgaggtaatagtcgggaggtagggaggagggagggtcgggggtcggggtggagggacggggtgggtaggttcggcgggtcgcgtttttaggagttcgcggagtcgagcgttaggttcgagcgatggtttcgatttcgtttattttttattttttttaaagttttttaagttcgtgtaagatcggcgtttgtttgttcgggattgtaaaattttttttcgcggtttagtcgtcgacgagggaggggtagacgaggggaggggagcggtcgtcgggtcgttgaggtttttagtgttggcggattttggggtagattggggtgttggaggcggggtgattttgtattgtaaatcgcgttttcgggtcggggcggtagaaatgagtcggcgggcgcggagttttgatttatcgcggtttcgagcgttcgtttcgttttcgtcgtgttttagatcgagtcgcggtatttacggatttaagattttaaaattaatcgagtaaacgaaattgtcgatttcgtttgggggaggtgcgggtagggtcggttcgggcggggtttgtttcgggttcgcgttcgcgttgacgcgcgtttggttttttatttttttttcgtagttttagtttcggaagtcgagcgagcggtcggcgcgtttatcgtcggggagttcgttaggtggatcggttcgcgtttcgtttttagcgagtcggggatttatttattatttttcgtatcgtcgacgtcgtttcgttcgttcggtttagtttgattaatgtcgcggtggaaacgggtttggagttggttttataagggttggcggtttttttcgacgtcgtttttttttatagtttttcgattgtagtggggcgg

SEQ ID NO: 6 - DNA, 1066 bp, Homo sapiens
gaaaaactttgggggaaatgaagagtgagcgaaatcgaagccatcgctcgggcctggcgctcggctccgcgggctcctgggggcgcgacccgccgggcctgcccaccccgtccctccaccccggcccccggccctccctcctccctgcctcccggctgttacctcataggtcgagggcgctcagtagccccctaaccagctggagaagtcgagtagctcgcgctccgcaggactcagcgcgccttcgcagccgctgtcgtccgacgagtaggcggaacgcggggagccgggctccgagctgcccccgcggcccggggacgaagaagcgcgggagggcgaggcggcgaccggggtggtccctggcggcccgcggggcgcagacggccgcacggcctgcggcctcagccctcccgccagcgcgttgcgcacggcgtcgtgctcggccagcaggcgctgcagcgcgcggatgtactccacggctgagcgcagcgtctccaccttgctcagcttcttgctggcgccgccgtgcggcacgtgctgccgcagcgcctggaagcccaagttcaccagcttcacgcggttgcgctcgcgctcattgcgccgcgctacggccgctgcgccgcctccggtctctgcggtggccggtcgccgccgccggctgcagcgcaacagttccggggacgcgggtctccgccgggcagcgcagccgacagggacggggggcgcagggggcgcggacctgggcagtgtgccgccgtccatcgcgcctgcatccacccgcccgctccaggtcccggcgcgccgcaggaaggtgcaggcagaggaaccggaggcgacggggaaaactgtggcgccccaagggggcttctggcacggcgccgccaggcaactccccagggcacgcgtcctaggtcgtctggagcccggggataggaggcctagtggtggcaggccgtacgcgccagggagcgtgggacgctcgtgtcccgcgcgtgcggccggactctcccaggtctccgcaggcgcggcgcaggcggctggtttttaaatgtatagataaccctcc

SEQ ID NO: 7 - DNA, 1066 bp, Artificial Sequence
  converted
gaaaaattttgggggaaatgaagagtgagcgaaatcgaagttatcgttcgggtttggcgttcggtttcgcgggtttttgggggcgcgattcgtcgggtttgtttatttcgtttttttatttcggttttcggtttttttttttttttgtttttcggttgttattttataggtcgagggcgtttagtagttttttaattagttggagaagtcgagtagttcgcgtttcgtaggatttagcgcgttttcgtagtcgttgtcgttcgacgagtaggcggaacgcggggagtcgggtttcgagttgttttcgcggttcggggacgaagaagcgcgggagggcgaggcggcgatcggggtggtttttggcggttcgcggggcgtagacggtcgtacggtttgcggttttagttttttcgttagcgcgttgcgtacggcgtcgtgttcggttagtaggcgttgtagcgcgcggatgtattttacggttgagcgtagcgtttttattttgtttagttttttgttggcgtcgtcgtgcggtacgtgttgtcgtagcgtttggaagtttaagtttattagttttacgcggttgcgttcgcgtttattgcgtcgcgttacggtcgttgcgtcgttttcggtttttgcggtggtcggtcgtcgtcgtcggttgtagcgtaatagtttcggggacgcgggttttcgtcgggtagcgtagtcgatagggacggggggcgtagggggcgcggatttgggtagtgtgtcgtcgtttatcgcgtttgtatttattcgttcgttttaggtttcggcgcgtcgtaggaaggtgtaggtagaggaatcggaggcgacggggaaaattgtggcgttttaagggggtttttggtacggcgtcgttaggtaattttttagggtacgcgttttaggtcgtttggagttcggggataggaggtttagtggtggtaggtcgtacgcgttagggagcgtgggacgttcgtgtttcgcgcgtgcggtcggatttttttaggttttcgtaggcgcggcgtaggcggttggtttttaaatgtatagataatttttt

SEQ ID NO: 8 - DNA, 1066 bp, Artificial Sequence
  converted
aaaaaattatctatacatttaaaaaccaaccgcctacgccgcgcctacgaaaacctaaaaaaatccgaccgcacgcgcgaaacacgaacgtcccacgctccctaacgcgtacgacctaccaccactaaacctcctatccccgaactccaaacgacctaaaacgcgtaccctaaaaaattacctaacgacgccgtaccaaaaacccccttaaaacgccacaattttccccgtcgcctccgattcctctacctacaccttcctacgacgcgccgaaacctaaaacgaacgaataaatacaaacgcgataaacgacgacacactacccaaatccgcgccccctacgccccccgtccctatcgactacgctacccgacgaaaacccgcgtccccgaaactattacgctacaaccgacgacgacgaccgaccaccgcaaaaaccgaaaacgacgcaacgaccgtaacgcgacgcaataaacgcgaacgcaaccgcgtaaaactaataaacttaaacttccaaacgctacgacaacacgtaccgcacgacgacgccaacaaaaaactaaacaaaataaaaacgctacgctcaaccgtaaaatacatccgcgcgctacaacgcctactaaccgaacacgacgccgtacgcaacgcgctaacgaaaaaactaaaaccgcaaaccgtacgaccgtctacgccccgcgaaccgccaaaaaccaccccgatcgccgcctcgccctcccgcgcttcttcgtccccgaaccgcgaaaacaactcgaaacccgactccccgcgttccgcctactcgtcgaacgacaacgactacgaaaacgcgctaaatcctacgaaacgcgaactactcgacttctccaactaattaaaaaactactaaacgccctcgacctataaaataacaaccgaaaaacaaaaaaaaaaaaaaaccgaaaaccgaaataaaaaaacgaaataaacaaacccgacgaatcgcgcccccaaaaacccgcgaaaccgaacgccaaacccgaacgataacttcgatttcgctcactcttcatttcccccaaaatttttc

SEQ ID NO: 9 - DNA, 1066 bp, Artificial Sequence
  converted
aaaaaactttaaaaaaaataaaaaataaacgaaatcgaaaccatcgctcgaacctaacgctcgactccgcgaactcctaaaaacgcgacccgccgaacctacccaccccgtccctccaccccgacccccgaccctccctcctccctacctcccgactattacctcataaatcgaaaacgctcaataaccccctaaccaactaaaaaaatcgaataactcgcgctccgcaaaactcaacgcgccttcgcaaccgctatcgtccgacgaataaacgaaacgcgaaaaaccgaactccgaactacccccgcgacccgaaaacgaaaaaacgcgaaaaaacgaaacgacgaccgaaataatccctaacgacccgcgaaacgcaaacgaccgcacgacctacgacctcaaccctcccgccaacgcgttacgcacgacgtcgtactcgaccaacaaacgctacaacgcgcgaatatactccacgactaaacgcaacgtctccaccttactcaacttcttactaacgccgccgtacgacacgtactaccgcaacgcctaaaaacccaaattcaccaacttcacgcgattacgctcgcgctcattacgccgcgctacgaccgctacgccgcctccgatctctacgataaccgatcgccgccgccgactacaacgcaacaattccgaaaacgcgaatctccgccgaacaacgcaaccgacaaaaacgaaaaacgcaaaaaacgcgaacctaaacaatataccgccgtccatcgcgcctacatccacccgcccgctccaaatcccgacgcgccgcaaaaaaatacaaacaaaaaaaccgaaaacgacgaaaaaaactataacgccccaaaaaaacttctaacacgacgccgccaaacaactccccaaaacacgcgtcctaaatcgtctaaaacccgaaaataaaaaacctaataataacaaaccgtacgcgccaaaaaacgtaaaacgctcgtatcccgcgcgtacgaccgaactctcccaaatctccgcaaacgcgacgcaaacgactaatttttaaatatataaataaccctcc

SEQ ID NO: 10 - DNA, 1066 bp, Artificial Sequence
  converted
ggagggttatttatatatttaaaaattagtcgtttgcgtcgcgtttgcggagatttgggagagttcggtcgtacgcgcgggatacgagcgttttacgttttttggcgcgtacggtttgttattattaggttttttattttcgggttttagacgatttaggacgcgtgttttggggagttgtttggcggcgtcgtgttagaagtttttttggggcgttatagtttttttcgtcgttttcggtttttttgtttgtatttttttgcggcgcgtcgggatttggagcgggcgggtggatgtaggcgcgatggacggcggtatattgtttaggttcgcgttttttgcgtttttcgtttttgtcggttgcgttgttcggcggagattcgcgttttcggaattgttgcgttgtagtcggcggcggcgatcggttatcgtagagatcggaggcggcgtagcggtcgtagcgcggcgtaatgagcgcgagcgtaatcgcgtgaagttggtgaatttgggtttttaggcgttgcggtagtacgtgtcgtacggcggcgttagtaagaagttgagtaaggtggagacgttgcgtttagtcgtggagtatattcgcgcgttgtagcgtttgttggtcgagtacgacgtcgtgcgtaacgcgttggcgggagggttgaggtcgtaggtcgtgcggtcgtttgcgtttcgcgggtcgttagggattatttcggtcgtcgtttcgttttttcgcgttttttcgttttcgggtcgcgggggtagttcggagttcggtttttcgcgtttcgtttattcgtcggacgatagcggttgcgaaggcgcgttgagttttgcggagcgcgagttattcgatttttttagttggttagggggttattgagcgttttcgatttatgaggtaatagtcgggaggtagggaggagggagggtcgggggtcggggtggagggacggggtgggtaggttcggcgggtcgcgtttttaggagttcgcggagtcgagcgttaggttcgagcgatggtttcgatttcgtttattttttattttttttaaagtttttt

SEQ ID NO: 11 - DNA, 66 bp, Homo sapiens
tgctcagcttcttgctggcgccgccgtgcggcacgtgctgccgcagcgcctggaagcccaagttca

SEQ ID NO: 12 - DNA, 66 bp, Artificial Sequence
  converted
tgtttagttttttgttggcgtcgtcgtgcggtacgtgttgtcgtagcgtttggaagtttaagttta

SEQ ID NO: 13 - DNA, 66 bp, Artificial Sequence
  converted
taaacttaaacttccaaacgctacgacaacacgtaccgcacgacgacgccaacaaaaaactaaaca

SEQ ID NO: 14 - DNA, 66 bp, Artificial Sequence
  converted
tactcaacttcttactaacgccgccgtacgacacgtactaccgcaacgcctaaaaacccaaattca

SEQ ID NO: 15 - DNA, 66 bp, Artificial Sequence
  converted
tgaatttgggtttttaggcgttgcggtagtacgtgtcgtacggcggcgttagtaagaagttgagta

SEQ ID NO: 16 - DNA, 294 bp, Homo sapiens
ccgccagggcacggccccccctgcgccccaaactgagcggcaaagtcagggcccgcggccggatgctcagagctaaaggccgcggaggacagatgtgcttcttcctccttcccgcgtctccccatacaagtactaccccgcacgtcccatcaggcttgcctgtgggccaggattcagggtcctgagccgaaacctaccaggagagagaaggctctggagacctctgtaacagtcgtgcggagaagacaaagtcagctgcgtgcgtctcctccggcgccggctctgcaaggaggg

SEQ ID NO: 17 - DNA, 294 bp, Artificial Sequence
  converted
tcgttagggtacggttttttttgcgttttaaattgagcggtaaagttagggttcgcggtcggatgtttagagttaaaggtcgcggaggatagatgtgtttttttttttttttcgcgtttttttatataagtattatttcgtacgttttattaggtttgtttgtgggttaggatttagggttttgagtcgaaatttattaggagagagaaggttttggagatttttgtaatagtcgtgcggagaagataaagttagttgcgtgcgtttttttcggcgtcggttttgtaaggaggg

SEQ ID NO: 18 - DNA, 294 bp, Artificial Sequence
  converted
ccctccttacaaaaccgacgccgaaaaaaacgcacgcaactaactttatcttctccgcacgactattacaaaaatctccaaaaccttctctctcctaataaatttcgactcaaaaccctaaatcctaacccacaaacaaacctaataaaacgtacgaaataatacttatataaaaaaacgcgaaaaaaaaaaaaaaacacatctatcctccgcgacctttaactctaaacatccgaccgcgaaccctaactttaccgctcaatttaaaacgcaaaaaaaaccgtaccctaacga

SEQ ID NO: 19 - DNA, 294 bp, Artificial Sequence
  converted
ccgccaaaacacgaccccccctacgccccaaactaaacgacaaaatcaaaacccgcgaccgaatactcaaaactaaaaaccgcgaaaaacaaatatacttcttcctccttcccgcgtctccccatacaaatactaccccgcacgtcccatcaaacttacctataaaccaaaattcaaaatcctaaaccgaaacctaccaaaaaaaaaaaaactctaaaaacctctataacaatcgtacgaaaaaaacaaaatcaactacgtacgtctcctccgacgccgactctacaaaaaaaa

SEQ ID NO: 20 - DNA, 294 bp, Artificial Sequence
  converted
ttttttttgtagagtcggcgtcggaggagacgtacgtagttgattttgtttttttcgtacgattgttatagaggtttttagagtttttttttttttggtaggtttcggtttaggattttgaattttggtttataggtaagtttgatgggacgtgcggggtagtatttgtatggggagacgcgggaaggaggaagaagtatatttgtttttcgcggtttttagttttgagtattcggtcgcgggttttgattttgtcgtttagtttggggcgtagggggggtcgtgttttggcgg

SEQ ID NO: 21 - DNA, 1080 bp, Homo sapiens
tcatctaggtgcctttctcctcatcccagcgagtgcaagggcatttctctattaccgtggcaatatattcaaatattcgaggtaaagatcgtccagccttcataggccacctggttccgtccccctcccccagctactcgggcacgtgtcccgactcccgccagcaggccgtgggggtgatgtggccgccatgaacccaccaaggacaagtagggcctggttttagctgcaaatgcaacaagtctctccgctgagccgcagctgctaccctctgccttctgctcctccgccagggcacggccccccctgcgccccaaactgagcggcaaagtcagggcccgcggccggatgctcagagctaaaggccgcggaggacagatgtgcttcttcctccttcccgcgtctccccatacaagtactaccccgcacgtcccatcaggcttgcctgtgggccaggattcagggtcctgagccgaaacctaccaggagagagaaggctctggagacctctgtaacagtcgtgcggagaagacaaagtcagctgcgtgcgtctcctccggcgccggctctgcaaggagggaggagggggcggggccccacaaccaccgcagcctcttaggtttctccattgtgattggaaatccgagtaccgcctacccctcccctcggctcgctcaatccccttcaaggacaagggctcaagtatgcacactccctcccccaccctccagatgaggaggtctccttccagaggcttccgctctcagctggtctcacatctcgcaggctcttcaatgcggaggccaagttatttataaaacttgcccatccccgactcttctggactttaacgctcttgttctattagatttagtacaaatagaaataaatttctttccttcttttttttattttgagacggagtctcactctgtcgcccaggctggagtgcagtggcgcgatctcggctcactgcaagctccgcctcccgggctcacgacattctcctgcctcagcctcccgagtagctgggactacaggcgcccgccacctcgcccggctaattttttgtatttttag

SEQ ID NO: 22 - DNA, 1080 bp, Artificial Sequence
  converted
ttatttaggtgtttttttttttattttagcgagtgtaagggtatttttttattatcgtggtaatatatttaaatattcgaggtaaagatcgtttagtttttataggttatttggtttcgttttttttttttagttattcgggtacgtgtttcgattttcgttagtaggtcgtgggggtgatgtggtcgttatgaatttattaaggataagtagggtttggttttagttgtaaatgtaataagttttttcgttgagtcgtagttgttattttttgttttttgttttttcgttagggtacggttttttttgcgttttaaattgagcggtaaagttagggttcgcggtcggatgtttagagttaaaggtcgcggaggatagatgtgtttttttttttttttcgcgtttttttatataagtattatttcgtacgttttattaggtttgtttgtgggttaggatttagggttttgagtcgaaatttattaggagagagaaggttttggagatttttgtaatagtcgtgcggagaagataaagttagttgcgtgcgtttttttcggcgtcggttttgtaaggagggaggagggggcggggttttataattatcgtagttttttaggtttttttattgtgattggaaattcgagtatcgtttattttttttttcggttcgtttaattttttttaaggataagggtttaagtatgtatatttttttttttattttttagatgaggaggtttttttttagaggttttcgtttttagttggttttatatttcgtaggttttttaatgcggaggttaagttatttataaaatttgtttattttcgatttttttggattttaacgtttttgttttattagatttagtataaatagaaataaattttttttttttttttttttattttgagacggagttttattttgtcgtttaggttggagtgtagtggcgcgatttcggtttattgtaagtttcgtttttcgggtttacgatatttttttgttttagtttttcgagtagttgggattataggcgttcgttatttcgttcggttaattttttgtatttttag

SEQ ID NO: 23 - DNA, 1080 bp, Artificial Sequence
  converted
ctaaaaatacaaaaaattaaccgaacgaaataacgaacgcctataatcccaactactcgaaaaactaaaacaaaaaaatatcgtaaacccgaaaaacgaaacttacaataaaccgaaatcgcgccactacactccaacctaaacgacaaaataaaactccgtctcaaaataaaaaaaaaaaaaaaaaaaatttatttctatttatactaaatctaataaaacaaaaacgttaaaatccaaaaaaatcgaaaataaacaaattttataaataacttaacctccgcattaaaaaacctacgaaatataaaaccaactaaaaacgaaaacctctaaaaaaaaacctcctcatctaaaaaataaaaaaaaaaatatacatacttaaacccttatccttaaaaaaaattaaacgaaccgaaaaaaaaaataaacgatactcgaatttccaatcacaataaaaaaacctaaaaaactacgataattataaaaccccgccccctcctccctccttacaaaaccgacgccgaaaaaaacgcacgcaactaactttatcttctccgcacgactattacaaaaatctccaaaaccttctctctcctaataaatttcgactcaaaaccctaaatcctaacccacaaacaaacctaataaaacgtacgaaataatacttatataaaaaaacgcgaaaaaaaaaaaaaaacacatctatcctccgcgacctttaactctaaacatccgaccgcgaaccctaactttaccgctcaatttaaaacgcaaaaaaaaccgtaccctaacgaaaaaacaaaaaacaaaaaataacaactacgactcaacgaaaaaacttattacatttacaactaaaaccaaaccctacttatccttaataaattcataacgaccacatcacccccacgacctactaacgaaaatcgaaacacgtacccgaataactaaaaaaaaaaaacgaaaccaaataacctataaaaactaaacgatctttacctcgaatatttaaatatattaccacgataataaaaaaatacccttacactcgctaaaataaaaaaaaaaacacctaaataa

SEQ ID NO: 24 - DNA, 1080 bp, Artificial Sequence
  converted
tcatctaaatacctttctcctcatcccaacgaatacaaaaacatttctctattaccgtaacaatatattcaaatattcgaaataaaaatcgtccaaccttcataaaccacctaattccgtccccctcccccaactactcgaacacgtatcccgactcccgccaacaaaccgtaaaaataatataaccgccataaacccaccaaaaacaaataaaacctaattttaactacaaatacaacaaatctctccgctaaaccgcaactactaccctctaccttctactcctccgccaaaacacgaccccccctacgccccaaactaaacgacaaaatcaaaacccgcgaccgaatactcaaaactaaaaaccgcgaaaaacaaatatacttcttcctccttcccgcgtctccccatacaaatactaccccgcacgtcccatcaaacttacctataaaccaaaattcaaaatcctaaaccgaaacctaccaaaaaaaaaaaaactctaaaaacctctataacaatcgtacgaaaaaaacaaaatcaactacgtacgtctcctccgacgccgactctacaaaaaaaaaaaaaaaaacgaaaccccacaaccaccgcaacctcttaaatttctccattataattaaaaatccgaataccgcctacccctcccctcgactcgctcaatccccttcaaaaacaaaaactcaaatatacacactccctcccccaccctccaaataaaaaaatctccttccaaaaacttccgctctcaactaatctcacatctcgcaaactcttcaatacgaaaaccaaattatttataaaacttacccatccccgactcttctaaactttaacgctcttattctattaaatttaatacaaataaaaataaatttctttccttcttttttttattttaaaacgaaatctcactctatcgcccaaactaaaatacaataacgcgatctcgactcactacaaactccgcctcccgaactcacgacattctcctacctcaacctcccgaataactaaaactacaaacgcccgccacctcgcccgactaattttttatatttttaa

SEQ ID NO: 25 - DNA, 1080 bp, Artificial Sequence
  converted
ttaaaaatataaaaaattagtcgggcgaggtggcgggcgtttgtagttttagttattcgggaggttgaggtaggagaatgtcgtgagttcgggaggcggagtttgtagtgagtcgagatcgcgttattgtattttagtttgggcgatagagtgagatttcgttttaaaataaaaaaaagaaggaaagaaatttatttttatttgtattaaatttaatagaataagagcgttaaagtttagaagagtcggggatgggtaagttttataaataatttggttttcgtattgaagagtttgcgagatgtgagattagttgagagcggaagtttttggaaggagatttttttatttggagggtgggggagggagtgtgtatatttgagtttttgtttttgaaggggattgagcgagtcgaggggaggggtaggcggtattcggatttttaattataatggagaaatttaagaggttgcggtggttgtggggtttcgtttttttttttttttttgtagagtcggcgtcggaggagacgtacgtagttgattttgtttttttcgtacgattgttatagaggtttttagagtttttttttttttggtaggtttcggtttaggattttgaattttggtttataggtaagtttgatgggacgtgcggggtagtatttgtatggggagacgcgggaaggaggaagaagtatatttgtttttcgcggtttttagttttgagtattcggtcgcgggttttgattttgtcgtttagtttggggcgtagggggggtcgtgttttggcggaggagtagaaggtagagggtagtagttgcggtttagcggagagatttgttgtatttgtagttaaaattaggttttatttgtttttggtgggtttatggcggttatattatttttacggtttgttggcgggagtcgggatacgtgttcgagtagttgggggagggggacggaattaggtggtttatgaaggttggacgatttttatttcgaatatttgaatatattgttacggtaatagagaaatgtttttgtattcgttgggatgaggagaaaggtatttagatga

SEQ ID NO: 26 - DNA, 80 bp, Homo sapiens
tggagacctctgtaacagtcgtgcggagaagacaaagtcagctgcgtgcgtctcctccggcgccggctctgcaaggaggg

SEQ ID NO: 27 - DNA, 80 bp, Artificial Sequence
  converted
tggagatttttgtaatagtcgtgcggagaagataaagttagttgcgtgcgtttttttcggcgtcggttttgtaaggaggg

SEQ ID NO: 28 - DNA, 80 bp, Artificial Sequence
  converted
ccctccttacaaaaccgacgccgaaaaaaacgcacgcaactaactttatcttctccgcacgactattacaaaaatctcca

SEQ ID NO: 29 - DNA, 80 bp, Artificial Sequence
  converted
taaaaacctctataacaatcgtacgaaaaaaacaaaatcaactacgtacgtctcctccgacgccgactctacaaaaaaaa

SEQ ID NO: 30 - DNA, 80 bp, Artificial Sequence
  converted
ttttttttgtagagtcggcgtcggaggagacgtacgtagttgattttgtttttttcgtacgattgttatagaggttttta

SEQ ID NO: 31 - DNA, 996 bp, Homo sapiens
gcgggggacagacgcggccgcagcccaggtcccgggagcgccacggaacctaacggtggcagcggaggtcgcgcccctcagtgcccgcgctctccccgtcgggagcttcctggtcgcccctgcggcggcggctcggggtgtctggccggcgcggggctcgcccagcctggtccggggagaggactggctgggcaggggcgccgccccgcctcgggagaggcgggccgggcggggctgggagtatttgaggctcggagccaccgccccgccggcgcccgcagcacctcctcgccagcagccgtccggagccagccaacgagcggtgagctgcgcggggcgcgggggacgcggctccggccgggcaggggagagggcgcccgggcgctgcttggggcgcggtccggagagggttcggctccccgggaccgggccggggcgcgcgcggagagccccacagcctgtgctctgccctccaggagcggggcggcgggcagcgatctgggcccggggcagtcgcctttgattatcgagggcgctggcgttcggggaaggttggcagcaccttacgagacccacacacgtccccggggcggcacgggccaccttctgcggagcctcgtgggcttcgccgccgtcgcacctccgccgcctgcgctctgcggccccagagtaagccccatccggtgacgagccgcagtctggtcaccccagtcccaccaggtcccgctgcgaggggaggcggaggggctcgctcagcaaaccagacggccgctccagtttctctaattggggttggagccccgtcacccttccccagatcacggccgcgggggagcgaagggacttcccaggactgcatagggcgcctcccgggacccacagcttggctggcccggccccttttgagcatcagctgaggctggacttggaggagatctcgctgtggagcgcgaatgagttcttccaccgggaccagctgcggccgggctgcagccg

SEQ ID NO: 32 - DNA, 996 bp, Artificial Sequence
  converted
gcgggggatagacgcggtcgtagtttaggtttcgggagcgttacggaatttaacggtggtagcggaggtcgcgttttttagtgttcgcgtttttttcgtcgggagttttttggtcgtttttgcggcggcggttcggggtgtttggtcggcgcggggttcgtttagtttggttcggggagaggattggttgggtaggggcgtcgtttcgtttcgggagaggcgggtcgggcggggttgggagtatttgaggttcggagttatcgtttcgtcggcgttcgtagtattttttcgttagtagtcgttcggagttagttaacgagcggtgagttgcgcggggcgcgggggacgcggtttcggtcgggtaggggagagggcgttcgggcgttgtttggggcgcggttcggagagggttcggtttttcgggatcgggtcggggcgcgcgcggagagttttatagtttgtgttttgttttttaggagcggggcggcgggtagcgatttgggttcggggtagtcgtttttgattatcgagggcgttggcgttcggggaaggttggtagtattttacgagatttatatacgttttcggggcggtacgggttattttttgcggagtttcgtgggtttcgtcgtcgtcgtattttcgtcgtttgcgttttgcggttttagagtaagttttattcggtgacgagtcgtagtttggttattttagttttattaggtttcgttgcgaggggaggcggaggggttcgtttagtaaattagacggtcgttttagtttttttaattggggttggagtttcgttatttttttttagattacggtcgcgggggagcgaagggattttttaggattgtatagggcgtttttcgggatttatagtttggttggttcggttttttttgagtattagttgaggttggatttggaggagatttcgttgtggagcgcgaatgagtttttttatcgggattagttgcggtcgggttgtagtcg

SEQ ID NO: 33 - DNA, 996 bp, Artificial Sequence
  converted
cgactacaacccgaccgcaactaatcccgataaaaaaactcattcgcgctccacaacgaaatctcctccaaatccaacctcaactaatactcaaaaaaaaccgaaccaaccaaactataaatcccgaaaaacgccctatacaatcctaaaaaatcccttcgctcccccgcgaccgtaatctaaaaaaaaataacgaaactccaaccccaattaaaaaaactaaaacgaccgtctaatttactaaacgaacccctccgcctcccctcgcaacgaaacctaataaaactaaaataaccaaactacgactcgtcaccgaataaaacttactctaaaaccgcaaaacgcaaacgacgaaaatacgacgacgacgaaacccacgaaactccgcaaaaaataacccgtaccgccccgaaaacgtatataaatctcgtaaaatactaccaaccttccccgaacgccaacgccctcgataatcaaaaacgactaccccgaacccaaatcgctacccgccgccccgctcctaaaaaacaaaacacaaactataaaactctccgcgcgcgccccgacccgatcccgaaaaaccgaaccctctccgaaccgcgccccaaacaacgcccgaacgccctctcccctacccgaccgaaaccgcgtcccccgcgccccgcgcaactcaccgctcgttaactaactccgaacgactactaacgaaaaaatactacgaacgccgacgaaacgataactccgaacctcaaatactcccaaccccgcccgacccgcctctcccgaaacgaaacgacgcccctacccaaccaatcctctccccgaaccaaactaaacgaaccccgcgccgaccaaacaccccgaaccgccgccgcaaaaacgaccaaaaaactcccgacgaaaaaaacgcgaacactaaaaaacgcgacctccgctaccaccgttaaattccgtaacgctcccgaaacctaaactacgaccgcgtctatcccccgc

SEQ ID NO: 34 - DNA, 996 bp, Artificial Sequence
  converted
acgaaaaacaaacgcgaccgcaacccaaatcccgaaaacgccacgaaacctaacgataacaacgaaaatcgcgcccctcaatacccgcgctctccccgtcgaaaacttcctaatcgcccctacgacgacgactcgaaatatctaaccgacgcgaaactcgcccaacctaatccgaaaaaaaaactaactaaacaaaaacgccgccccgcctcgaaaaaaacgaaccgaacgaaactaaaaatatttaaaactcgaaaccaccgccccgccgacgcccgcaacacctcctcgccaacaaccgtccgaaaccaaccaacgaacgataaactacgcgaaacgcgaaaaacgcgactccgaccgaacaaaaaaaaaaacgcccgaacgctacttaaaacgcgatccgaaaaaaattcgactccccgaaaccgaaccgaaacgcgcgcgaaaaaccccacaacctatactctaccctccaaaaacgaaacgacgaacaacgatctaaacccgaaacaatcgcctttaattatcgaaaacgctaacgttcgaaaaaaattaacaacaccttacgaaacccacacacgtccccgaaacgacacgaaccaccttctacgaaacctcgtaaacttcgccgccgtcgcacctccgccgcctacgctctacgaccccaaaataaaccccatccgataacgaaccgcaatctaatcaccccaatcccaccaaatcccgctacgaaaaaaaacgaaaaaactcgctcaacaaaccaaacgaccgctccaatttctctaattaaaattaaaaccccgtcacccttccccaaatcacgaccgcgaaaaaacgaaaaaacttcccaaaactacataaaacgcctcccgaaacccacaacttaactaacccgaccccttttaaacatcaactaaaactaaacttaaaaaaaatctcgctataaaacgcgaataaattcttccaccgaaaccaactacgaccgaactacaaccg

SEQ ID NO: 35 - DNA, 996 bp, Artificial Sequence
  converted
cggttgtagttcggtcgtagttggtttcggtggaagaatttattcgcgttttatagcgagattttttttaagtttagttttagttgatgtttaaaaggggtcgggttagttaagttgtgggtttcgggaggcgttttatgtagttttgggaagttttttcgttttttcgcggtcgtgatttggggaagggtgacggggttttaattttaattagagaaattggagcggtcgtttggtttgttgagcgagttttttcgttttttttcgtagcgggatttggtgggattggggtgattagattgcggttcgttatcggatggggtttattttggggtcgtagagcgtaggcggcggaggtgcgacggcggcgaagtttacgaggtttcgtagaaggtggttcgtgtcgtttcggggacgtgtgtgggtttcgtaaggtgttgttaatttttttcgaacgttagcgttttcgataattaaaggcgattgtttcgggtttagatcgttgttcgtcgtttcgtttttggagggtagagtataggttgtggggtttttcgcgcgcgtttcggttcggtttcggggagtcgaatttttttcggatcgcgttttaagtagcgttcgggcgtttttttttttgttcggtcggagtcgcgtttttcgcgtttcgcgtagtttatcgttcgttggttggtttcggacggttgttggcgaggaggtgttgcgggcgtcggcggggcggtggtttcgagttttaaatatttttagtttcgttcggttcgtttttttcgaggcggggcggcgtttttgtttagttagtttttttttcggattaggttgggcgagtttcgcgtcggttagatatttcgagtcgtcgtcgtaggggcgattaggaagttttcgacggggagagcgcgggtattgaggggcgcgattttcgttgttatcgttaggtttcgtggcgttttcgggatttgggttgcggtcgcgtttgtttttcgt

SEQ ID NO: 36 - DNA, 1099 bp, Homo sapiens
cctctcagcgaattcttcactcaggtgattctggagagggcgggggacagacgcggccgcagcccaggtcccgggagcgccacggaacctaacggtggcagcggaggtcgcgcccctcagtgcccgcgctctccccgtcgggagcttcctggtcgcccctgcggcggcggctcggggtgtctggccggcgcggggctcgcccagcctggtccggggagaggactggctgggcaggggcgccgccccgcctcgggagaggcgggccgggcggggctgggagtatttgaggctcggagccaccgccccgccggcgcccgcagcacctcctcgccagcagccgtccggagccagccaacgagcggtgagctgcgcggggcgcgggggacgcggctccggccgggcaggggagagggcgcccgggcgctgcttggggcgcggtccggagagggttcggctccccgggaccgggccggggcgcgcgcggagagccccacagcctgtgctctgccctccaggagcggggcggcgggcagcgatctgggcccggggcagtcgcctttgattatcgagggcgctggcgttcggggaaggttggcagcaccttacgagacccacacacgtccccggggcggcacgggccaccttctgcggagcctcgtgggcttcgccgccgtcgcacctccgccgcctgcgctctgcggccccagagtaagccccatccggtgacgagccgcagtctggtcaccccagtcccaccaggtcccgctgcgaggggaggcggaggggctcgctcagcaaaccagacggccgctccagtttctctaattggggttggagccccgtcacccttccccagatcacggccgcgggggagcgaagggacttcccaggactgcatagggcgcctcccgggacccacagcttggctggcccggccccttttgagcatcagctgaggctggacttggaggagatctcgctgtggagcgcgaatgagttcttccaccgggaccagctgcggccgggctgcagccgctgtctggttgctgatcccacagaaaatggtgcttgccccatggcctgagtgggcatgacccgg

SEQ ID NO: 37 - DNA, 1099 bp, Artificial Sequence
  converted
ttttttagcgaattttttatttaggtgattttggagagggcgggggatagacgcggtcgtagtttaggtttcgggagcgttacggaatttaacggtggtagcggaggtcgcgttttttagtgttcgcgtttttttcgtcgggagttttttggtcgtttttgcggcggcggttcggggtgtttggtcggcgcggggttcgtttagtttggttcggggagaggattggttgggtaggggcgtcgtttcgtttcgggagaggcgggtcgggcggggttgggagtatttgaggttcggagttatcgtttcgtcggcgttcgtagtattttttcgttagtagtcgttcggagttagttaacgagcggtgagttgcgcggggcgcgggggacgcggtttcggtcgggtaggggagagggcgttcgggcgttgtttggggcgcggttcggagagggttcggtttttcgggatcgggtcggggcgcgcgcggagagttttatagtttgtgttttgttttttaggagcggggcggcgggtagcgatttgggttcggggtagtcgtttttgattatcgagggcgttggcgttcggggaaggttggtagtattttacgagatttatatacgttttcggggcggtacgggttattttttgcggagtttcgtgggtttcgtcgtcgtcgtattttcgtcgtttgcgttttgcggttttagagtaagttttattcggtgacgagtcgtagtttggttattttagttttattaggtttcgttgcgaggggaggcggaggggttcgtttagtaaattagacggtcgttttagtttttttaattggggttggagtttcgttatttttttttagattacggtcgcgggggagcgaagggattttttaggattgtatagggcgtttttcgggatttatagtttggttggttcggttttttttgagtattagttgaggttggatttggaggagatttcgttgtggagcgcgaatgagtttttttatcgggattagttgcggtcgggttgtagtcgttgtttggttgttgattttatagaaaatggtgtttgttttatggtttgagtgggtatgattcgg

SEQ ID NO: 38 - DNA, 1099 bp, Artificial Sequence
  converted
ccgaatcatacccactcaaaccataaaacaaacaccattttctataaaatcaacaaccaaacaacgactacaacccgaccgcaactaatcccgataaaaaaactcattcgcgctccacaacgaaatctcctccaaatccaacctcaactaatactcaaaaaaaaccgaaccaaccaaactataaatcccgaaaaacgccctatacaatcctaaaaaatcccttcgctcccccgcgaccgtaatctaaaaaaaaataacgaaactccaaccccaattaaaaaaactaaaacgaccgtctaatttactaaacgaacccctccgcctcccctcgcaacgaaacctaataaaactaaaataaccaaactacgactcgtcaccgaataaaacttactctaaaaccgcaaaacgcaaacgacgaaaatacgacgacgacgaaacccacgaaactccgcaaaaaataacccgtaccgccccgaaaacgtatataaatctcgtaaaatactaccaaccttccccgaacgccaacgccctcgataatcaaaaacgactaccccgaacccaaatcgctacccgccgccccgctcctaaaaaacaaaacacaaactataaaactctccgcgcgcgccccgacccgatcccgaaaaaccgaaccctctccgaaccgcgccccaaacaacgcccgaacgccctctcccctacccgaccgaaaccgcgtcccccgcgccccgcgcaactcaccgctcgttaactaactccgaacgactactaacgaaaaaatactacgaacgccgacgaaacgataactccgaacctcaaatactcccaaccccgcccgacccgcctctcccgaaacgaaacgacgcccctacccaaccaatcctctccccgaaccaaactaaacgaaccccgcgccgaccaaacaccccgaaccgccgccgcaaaaacgaccaaaaaactcccgacgaaaaaaacgcgaacactaaaaaacgcgacctccgctaccaccgttaaattccgtaacgctcccgaaacctaaactacgaccgcgtctatcccccgccctctccaaaatcacctaaataaaaaattcgctaaaaaa

SEQ ID NO: 39 - DNA, 1099 bp, Artificial Sequence
  converted
cctctcaacgaattcttcactcaaataattctaaaaaaaacgaaaaacaaacgcgaccgcaacccaaatcccgaaaacgccacgaaacctaacgataacaacgaaaatcgcgcccctcaatacccgcgctctccccgtcgaaaacttcctaatcgcccctacgacgacgactcgaaatatctaaccgacgcgaaactcgcccaacctaatccgaaaaaaaaactaactaaacaaaaacgccgccccgcctcgaaaaaaacgaaccgaacgaaactaaaaatatttaaaactcgaaaccaccgccccgccgacgcccgcaacacctcctcgccaacaaccgtccgaaaccaaccaacgaacgataaactacgcgaaacgcgaaaaacgcgactccgaccgaacaaaaaaaaaaacgcccgaacgctacttaaaacgcgatccgaaaaaaattcgactccccgaaaccgaaccgaaacgcgcgcgaaaaaccccacaacctatactctaccctccaaaaacgaaacgacgaacaacgatctaaacccgaaacaatcgcctttaattatcgaaaacgctaacgttcgaaaaaaattaacaacaccttacgaaacccacacacgtccccgaaacgacacgaaccaccttctacgaaacctcgtaaacttcgccgccgtcgcacctccgccgcctacgctctacgaccccaaaataaaccccatccgataacgaaccgcaatctaatcaccccaatcccaccaaatcccgctacgaaaaaaaacgaaaaaactcgctcaacaaaccaaacgaccgctccaatttctctaattaaaattaaaaccccgtcacccttccccaaatcacgaccgcgaaaaaacgaaaaaacttcccaaaactacataaaacgcctcccgaaacccacaacttaactaacccgaccccttttaaacatcaactaaaactaaacttaaaaaaaatctcgctataaaacgcgaataaattcttccaccgaaaccaactacgaccgaactacaaccgctatctaattactaatcccacaaaaaataatacttaccccataacctaaataaacataacccga

SEQ ID NO: 40 - DNA, 1099 bp, Artificial Sequence
  converted
tcgggttatgtttatttaggttatggggtaagtattattttttgtgggattagtaattagatagcggttgtagttcggtcgtagttggtttcggtggaagaatttattcgcgttttatagcgagattttttttaagtttagttttagttgatgtttaaaaggggtcgggttagttaagttgtgggtttcgggaggcgttttatgtagttttgggaagttttttcgttttttcgcggtcgtgatttggggaagggtgacggggttttaattttaattagagaaattggagcggtcgtttggtttgttgagcgagttttttcgttttttttcgtagcgggatttggtgggattggggtgattagattgcggttcgttatcggatggggtttattttggggtcgtagagcgtaggcggcggaggtgcgacggcggcgaagtttacgaggtttcgtagaaggtggttcgtgtcgtttcggggacgtgtgtgggtttcgtaaggtgttgttaatttttttcgaacgttagcgttttcgataattaaaggcgattgtttcgggtttagatcgttgttcgtcgtttcgtttttggagggtagagtataggttgtggggtttttcgcgcgcgtttcggttcggtttcggggagtcgaatttttttcggatcgcgttttaagtagcgttcgggcgtttttttttttgttcggtcggagtcgcgtttttcgcgtttcgcgtagtttatcgttcgttggttggtttcggacggttgttggcgaggaggtgttgcgggcgtcggcggggcggtggtttcgagttttaaatatttttagtttcgttcggttcgtttttttcgaggcggggcggcgtttttgtttagttagtttttttttcggattaggttgggcgagtttcgcgtcggttagatatttcgagtcgtcgtcgtaggggcgattaggaagttttcgacggggagagcgcgggtattgaggggcgcgattttcgttgttatcgttaggtttcgtggcgttttcgggatttgggttgcggtcgcgtttgtttttcgttttttttagaattatttgagtgaagaattcgttgagagg

SEQ ID NO: 41 - DNA, 99 bp, Homo sapiens
tgctctgccctccaggagcggggcggcgggcagcgatctgggcccggggcagtcgcctttgattatcgagggcgctggcgttcggggaaggttggcagc

SEQ ID NO: 42 - DNA, 99 bp, Artificial Sequence
  converted
tgttttgttttttaggagcggggcggcgggtagcgatttgggttcggggtagtcgtttttgattatcgagggcgttggcgttcggggaaggttggtagt

SEQ ID NO: 43 - DNA, 99 bp, Artificial Sequence
  converted
actaccaaccttccccgaacgccaacgccctcgataatcaaaaacgactaccccgaacccaaatcgctacccgccgccccgctcctaaaaaacaaaaca

SEQ ID NO: 44 - DNA, 99 bp, Artificial Sequence
  converted
tactctaccctccaaaaacgaaacgacgaacaacgatctaaacccgaaacaatcgcctttaattatcgaaaacgctaacgttcgaaaaaaattaacaac

SEQ ID NO: 45 - DNA, 99 bp, Artificial Sequence
  converted
gttgttaatttttttcgaacgttagcgttttcgataattaaaggcgattgtttcgggtttagatcgttgttcgtcgtttcgtttttggagggtagagta

SEQ ID NO: 46 - DNA, 1271 bp, Homo sapiens
ccgagtcccgggccagcggagtctaaacagcccggctcgggcggggctgggggcgggcccggccagggtaaataaagccccggaggcccgtgtgggtctctccggtgcccggggcgctttcggggtgatgtcatggctttcactctgacgtcaccttgggcatatgacgtcacgcgcagccaccacgcccagaggccagggccctcgcgccaaaaccgaggactttcggtggagccactggcgcgcccccacgacggcgggtaggggcctggggtgctgcagctcccggctaatccgggggtctccaccattccttctcgctcctctccctcaccccaccgcgacccccccagcggctgccggcgggggccggggaggggcggaaactcctccagggaacccggccccgcctcccgccccgcctcccgccgcagggccaggaagcgcggaaggaaccgccgggggccatggacggagcagtgatggaagggccgctttttttgcagagtcagcgctttgggaccaaggtagtctggcgcatggatgccgaaccttatccgggctagtagtgggtgagagagcccagaaacagggagaggtgggcagcgggctggagagcggcgccgggagttggagagcctgtgactttcccgtgaagttgctttgcacactcccgggaagcgtctgtagtctaggggttctggtcctggggagacggagtggcatcgtccttgggaaacttcgcccccaaccccgtttggaagccccagatcccaaatcgacttgcgccgcaacctccttccccgtcgggacccgggccgcctgcgcacgccactccctctcgagcactctctctctctccctagaggtggaggaagacctgggccgtgctctacccggccagtccccacggcgtagcgcggctcgagttctttgaccataaggggtcgagctctgggggtggccgagggagctcgcgccgcctggactgcaaagtgatccgtctggctgagtgtgtgagtgtggcccccgtcaccgtggagaccccccctgagcccggcgccactgccttccgcctggacactgctcagcgctcgcacctgctggcggccgacgcgccgtccagtgcagcctgggtgcagacgctgtgccgaaacgcctttccggtgaggagctgcggcgatgcggggtgggggcagttacagaggcagagaaatggggctgcctcagaccgacccccgctccccgctgaggaaattacgggtttctcg

SEQ ID NO: 47 - DNA, 1271 bp, Artificial Sequence
  converted
tcgagtttcgggttagcggagtttaaatagttcggttcgggcggggttgggggcgggttcggttagggtaaataaagtttcggaggttcgtgtgggttttttcggtgttcggggcgttttcggggtgatgttatggtttttattttgacgttattttgggtatatgacgttacgcgtagttattacgtttagaggttagggttttcgcgttaaaatcgaggattttcggtggagttattggcgcgtttttacgacggcgggtaggggtttggggtgttgtagttttcggttaattcgggggtttttattattttttttcgttttttttttttattttatcgcgatttttttagcggttgtcggcgggggtcggggaggggcggaaatttttttagggaattcggtttcgtttttcgtttcgtttttcgtcgtagggttaggaagcgcggaaggaatcgtcgggggttatggacggagtagtgatggaagggtcgttttttttgtagagttagcgttttgggattaaggtagtttggcgtatggatgtcgaattttattcgggttagtagtgggtgagagagtttagaaatagggagaggtgggtagcgggttggagagcggcgtcgggagttggagagtttgtgattttttcgtgaagttgttttgtatattttcgggaagcgtttgtagtttaggggttttggttttggggagacggagtggtatcgtttttgggaaatttcgtttttaatttcgtttggaagttttagattttaaatcgatttgcgtcgtaattttttttttcgtcgggattcgggtcgtttgcgtacgttattttttttcgagtattttttttttttttttagaggtggaggaagatttgggtcgtgttttattcggttagtttttacggcgtagcgcggttcgagttttttgattataaggggtcgagttttgggggtggtcgagggagttcgcgtcgtttggattgtaaagtgattcgtttggttgagtgtgtgagtgtggttttcgttatcgtggagattttttttgagttcggcgttattgtttttcgtttggatattgtttagcgttcgtatttgttggcggtcgacgcgtcgtttagtgtagtttgggtgtagacgttgtgtcgaaacgttttttcggtgaggagttgcggcgatgcggggtgggggtagttatagaggtagagaaatggggttgttttagatcgattttcgtttttcgttgaggaaattacgggtttttcg

SEQ ID NO: 48 - DNA, 1271 bp, Artificial Sequence
  converted
cgaaaaacccgtaatttcctcaacgaaaaacgaaaatcgatctaaaacaaccccatttctctacctctataactacccccaccccgcatcgccgcaactcctcaccgaaaaaacgtttcgacacaacgtctacacccaaactacactaaacgacgcgtcgaccgccaacaaatacgaacgctaaacaatatccaaacgaaaaacaataacgccgaactcaaaaaaaatctccacgataacgaaaaccacactcacacactcaaccaaacgaatcactttacaatccaaacgacgcgaactccctcgaccacccccaaaactcgaccccttataatcaaaaaactcgaaccgcgctacgccgtaaaaactaaccgaataaaacacgacccaaatcttcctccacctctaaaaaaaaaaaaaaaatactcgaaaaaaaataacgtacgcaaacgacccgaatcccgacgaaaaaaaaaattacgacgcaaatcgatttaaaatctaaaacttccaaacgaaattaaaaacgaaatttcccaaaaacgataccactccgtctccccaaaaccaaaacccctaaactacaaacgcttcccgaaaatatacaaaacaacttcacgaaaaaatcacaaactctccaactcccgacgccgctctccaacccgctacccacctctccctatttctaaactctctcacccactactaacccgaataaaattcgacatccatacgccaaactaccttaatcccaaaacgctaactctacaaaaaaaacgacccttccatcactactccgtccataacccccgacgattccttccgcgcttcctaaccctacgacgaaaaacgaaacgaaaaacgaaaccgaattccctaaaaaaatttccgcccctccccgacccccgccgacaaccgctaaaaaaatcgcgataaaataaaaaaaaaaaacgaaaaaaaataataaaaacccccgaattaaccgaaaactacaacaccccaaacccctacccgccgtcgtaaaaacgcgccaataactccaccgaaaatcctcgattttaacgcgaaaaccctaacctctaaacgtaataactacgcgtaacgtcatatacccaaaataacgtcaaaataaaaaccataacatcaccccgaaaacgccccgaacaccgaaaaaacccacacgaacctccgaaactttatttaccctaaccgaacccgcccccaaccccgcccgaaccgaactatttaaactccgctaacccgaaactcga

SEQ ID NO: 49 - DNA, 1271 bp, Artificial Sequence
  converted
ccgaatcccgaaccaacgaaatctaaacaacccgactcgaacgaaactaaaaacgaacccgaccaaaataaataaaaccccgaaaacccgtataaatctctccgatacccgaaacgctttcgaaataatatcataactttcactctaacgtcaccttaaacatataacgtcacgcgcaaccaccacgcccaaaaaccaaaaccctcgcgccaaaaccgaaaactttcgataaaaccactaacgcgcccccacgacgacgaataaaaacctaaaatactacaactcccgactaatccgaaaatctccaccattccttctcgctcctctccctcaccccaccgcgacccccccaacgactaccgacgaaaaccgaaaaaaaacgaaaactcctccaaaaaacccgaccccgcctcccgccccgcctcccgccgcaaaaccaaaaaacgcgaaaaaaaccgccgaaaaccataaacgaaacaataataaaaaaaccgctttttttacaaaatcaacgctttaaaaccaaaataatctaacgcataaataccgaaccttatccgaactaataataaataaaaaaacccaaaaacaaaaaaaaataaacaacgaactaaaaaacgacgccgaaaattaaaaaacctataactttcccgtaaaattactttacacactcccgaaaaacgtctataatctaaaaattctaatcctaaaaaaacgaaataacatcgtccttaaaaaacttcgcccccaaccccgtttaaaaaccccaaatcccaaatcgacttacgccgcaacctccttccccgtcgaaacccgaaccgcctacgcacgccactccctctcgaacactctctctctctccctaaaaataaaaaaaaacctaaaccgtactctacccgaccaatccccacgacgtaacgcgactcgaattctttaaccataaaaaatcgaactctaaaaataaccgaaaaaactcgcgccgcctaaactacaaaataatccgtctaactaaatatataaatataacccccgtcaccgtaaaaaccccccctaaacccgacgccactaccttccgcctaaacactactcaacgctcgcacctactaacgaccgacgcgccgtccaatacaacctaaatacaaacgctataccgaaacgcctttccgataaaaaactacgacgatacgaaataaaaacaattacaaaaacaaaaaaataaaactacctcaaaccgacccccgctccccgctaaaaaaattacgaatttctcg

SEQ ID NO: 50 - DNA, 1271 bp, Artificial Sequence
  converted
cgagaaattcgtaatttttttagcggggagcgggggtcggtttgaggtagttttatttttttgtttttgtaattgtttttatttcgtatcgtcgtagttttttatcggaaaggcgtttcggtatagcgtttgtatttaggttgtattggacggcgcgtcggtcgttagtaggtgcgagcgttgagtagtgtttaggcggaaggtagtggcgtcgggtttagggggggtttttacggtgacgggggttatatttatatatttagttagacggattattttgtagtttaggcggcgcgagttttttcggttatttttagagttcgattttttatggttaaagaattcgagtcgcgttacgtcgtggggattggtcgggtagagtacggtttaggtttttttttatttttagggagagagagagagtgttcgagagggagtggcgtgcgtaggcggttcgggtttcgacggggaaggaggttgcggcgtaagtcgatttgggatttggggtttttaaacggggttgggggcgaagttttttaaggacgatgttatttcgtttttttaggattagaatttttagattatagacgtttttcgggagtgtgtaaagtaattttacgggaaagttataggttttttaattttcggcgtcgttttttagttcgttgtttatttttttttgtttttgggtttttttatttattattagttcggataaggttcggtatttatgcgttagattattttggttttaaagcgttgattttgtaaaaaaagcggtttttttattattgtttcgtttatggttttcggcggtttttttcgcgttttttggttttgcggcgggaggcggggcgggaggcggggtcgggttttttggaggagttttcgttttttttcggttttcgtcggtagtcgttgggggggtcgcggtggggtgagggagaggagcgagaaggaatggtggagattttcggattagtcgggagttgtagtattttaggtttttattcgtcgtcgtgggggcgcgttagtggttttatcgaaagttttcggttttggcgcgagggttttggtttttgggcgtggtggttgcgcgtgacgttatatgtttaaggtgacgttagagtgaaagttatgatattatttcgaaagcgtttcgggtatcggagagatttatacgggttttcggggttttatttattttggtcgggttcgtttttagtttcgttcgagtcgggttgtttagatttcgttggttcgggattcgg

SEQ ID NO: 51 - DNA, 1082 bp, Homo sapiens
gtgtgggtctctccggtgcccggggcgctttcggggtgatgtcatggctttcactctgacgtcaccttgggcatatgacgtcacgcgcagccaccacgcccagaggccagggccctcgcgccaaaaccgaggactttcggtggagccactggcgcgcccccacgacggcgggtaggggcctggggtgctgcagctcccggctaatccgggggtctccaccattccttctcgctcctctccctcaccccaccgcgacccccccagcggctgccggcgggggccggggaggggcggaaactcctccagggaacccggccccgcctcccgccccgcctcccgccgcagggccaggaagcgcggaaggaaccgccgggggccatggacggagcagtgatggaagggccgctttttttgcagagtcagcgctttgggaccaaggtagtctggcgcatggatgccgaaccttatccgggctagtagtgggtgagagagcccagaaacagggagaggtgggcagcgggctggagagcggcgccgggagttggagagcctgtgactttcccgtgaagttgctttgcacactcccgggaagcgtctgtagtctaggggttctggtcctggggagacggagtggcatcgtccttgggaaacttcgcccccaaccccgtttggaagccccagatcccaaatcgacttgcgccgcaacctccttccccgtcgggacccgggccgcctgcgcacgccactccctctcgagcactctctctctctccctagaggtggaggaagacctgggccgtgctctacccggccagtccccacggcgtagcgcggctcgagttctttgaccataaggggtcgagctctgggggtggccgagggagctcgcgccgcctggactgcaaagtgatccgtctggctgagtgtgtgagtgtggcccccgtcaccgtggagaccccccctgagcccggcgccactgccttccgcctggacactgctcagcgctcgcacctgctggcggccgacgcgccgtccagtgcagcctgggtgcagacgctgtgccgaaacgcctttccggtgag

SEQ ID NO: 52 - DNA, 1082 bp, Artificial Sequence
  converted
gtgtgggttttttcggtgttcggggcgttttcggggtgatgttatggtttttattttgacgttattttgggtatatgacgttacgcgtagttattacgtttagaggttagggttttcgcgttaaaatcgaggattttcggtggagttattggcgcgtttttacgacggcgggtaggggtttggggtgttgtagttttcggttaattcgggggtttttattattttttttcgttttttttttttattttatcgcgatttttttagcggttgtcggcgggggtcggggaggggcggaaatttttttagggaattcggtttcgtttttcgtttcgtttttcgtcgtagggttaggaagcgcggaaggaatcgtcgggggttatggacggagtagtgatggaagggtcgttttttttgtagagttagcgttttgggattaaggtagtttggcgtatggatgtcgaattttattcgggttagtagtgggtgagagagtttagaaatagggagaggtgggtagcgggttggagagcggcgtcgggagttggagagtttgtgattttttcgtgaagttgttttgtatattttcgggaagcgtttgtagtttaggggttttggttttggggagacggagtggtatcgtttttgggaaatttcgtttttaatttcgtttggaagttttagattttaaatcgatttgcgtcgtaattttttttttcgtcgggattcgggtcgtttgcgtacgttattttttttcgagtattttttttttttttttagaggtggaggaagatttgggtcgtgttttattcggttagtttttacggcgtagcgcggttcgagttttttgattataaggggtcgagttttgggggtggtcgagggagttcgcgtcgtttggattgtaaagtgattcgtttggttgagtgtgtgagtgtggttttcgttatcgtggagattttttttgagttcggcgttattgtttttcgtttggatattgtttagcgttcgtatttgttggcggtcgacgcgtcgtttagtgtagtttgggtgtagacgttgtgtcgaaacgttttttcggtgag

SEQ ID NO: 53 - DNA, 1082 bp, Artificial Sequence
  converted
ctcaccgaaaaaacgtttcgacacaacgtctacacccaaactacactaaacgacgcgtcgaccgccaacaaatacgaacgctaaacaatatccaaacgaaaaacaataacgccgaactcaaaaaaaatctccacgataacgaaaaccacactcacacactcaaccaaacgaatcactttacaatccaaacgacgcgaactccctcgaccacccccaaaactcgaccccttataatcaaaaaactcgaaccgcgctacgccgtaaaaactaaccgaataaaacacgacccaaatcttcctccacctctaaaaaaaaaaaaaaaatactcgaaaaaaaataacgtacgcaaacgacccgaatcccgacgaaaaaaaaaattacgacgcaaatcgatttaaaatctaaaacttccaaacgaaattaaaaacgaaatttcccaaaaacgataccactccgtctccccaaaaccaaaacccctaaactacaaacgcttcccgaaaatatacaaaacaacttcacgaaaaaatcacaaactctccaactcccgacgccgctctccaacccgctacccacctctccctatttctaaactctctcacccactactaacccgaataaaattcgacatccatacgccaaactaccttaatcccaaaacgctaactctacaaaaaaaacgacccttccatcactactccgtccataacccccgacgattccttccgcgcttcctaaccctacgacgaaaaacgaaacgaaaaacgaaaccgaattccctaaaaaaatttccgcccctccccgacccccgccgacaaccgctaaaaaaatcgcgataaaataaaaaaaaaaaacgaaaaaaaataataaaaacccccgaattaaccgaaaactacaacaccccaaacccctacccgccgtcgtaaaaacgcgccaataactccaccgaaaatcctcgattttaacgcgaaaaccctaacctctaaacgtaataactacgcgtaacgtcatatacccaaaataacgtcaaaataaaaaccataacatcaccccgaaaacgccccgaacaccgaaaaaacccacac

SEQ ID NO: 54 - DNA, 1082 bp, Artificial Sequence
  converted
atataaatctctccgatacccgaaacgctttcgaaataatatcataactttcactctaacgtcaccttaaacatataacgtcacgcgcaaccaccacgcccaaaaaccaaaaccctcgcgccaaaaccgaaaactttcgataaaaccactaacgcgcccccacgacgacgaataaaaacctaaaatactacaactcccgactaatccgaaaatctccaccattccttctcgctcctctccctcaccccaccgcgacccccccaacgactaccgacgaaaaccgaaaaaaaacgaaaactcctccaaaaaacccgaccccgcctcccgccccgcctcccgccgcaaaaccaaaaaacgcgaaaaaaaccgccgaaaaccataaacgaaacaataataaaaaaaccgctttttttacaaaatcaacgctttaaaaccaaaataatctaacgcataaataccgaaccttatccgaactaataataaataaaaaaacccaaaaacaaaaaaaaataaacaacgaactaaaaaacgacgccgaaaattaaaaaacctataactttcccgtaaaattactttacacactcccgaaaaacgtctataatctaaaaattctaatcctaaaaaaacgaaataacatcgtccttaaaaaacttcgcccccaaccccgtttaaaaaccccaaatcccaaatcgacttacgccgcaacctccttccccgtcgaaacccgaaccgcctacgcacgccactccctctcgaacactctctctctctccctaaaaataaaaaaaaacctaaaccgtactctacccgaccaatccccacgacgtaacgcgactcgaattctttaaccataaaaaatcgaactctaaaaataaccgaaaaaactcgcgccgcctaaactacaaaataatccgtctaactaaatatataaatataacccccgtcaccgtaaaaaccccccctaaacccgacgccactaccttccgcctaaacactactcaacgctcgcacctactaacgaccgacgcgccgtccaatacaacctaaatacaaacgctataccgaaacgcctttccgataaa

SEQ ID NO: 55 - DNA, 1082 bp, Artificial Sequence
  converted
tttatcggaaaggcgtttcggtatagcgtttgtatttaggttgtattggacggcgcgtcggtcgttagtaggtgcgagcgttgagtagtgtttaggcggaaggtagtggcgtcgggtttagggggggtttttacggtgacgggggttatatttatatatttagttagacggattattttgtagtttaggcggcgcgagttttttcggttatttttagagttcgattttttatggttaaagaattcgagtcgcgttacgtcgtggggattggtcgggtagagtacggtttaggtttttttttatttttagggagagagagagagtgttcgagagggagtggcgtgcgtaggcggttcgggtttcgacggggaaggaggttgcggcgtaagtcgatttgggatttggggtttttaaacggggttgggggcgaagttttttaaggacgatgttatttcgtttttttaggattagaatttttagattatagacgtttttcgggagtgtgtaaagtaattttacgggaaagttataggttttttaattttcggcgtcgttttttagttcgttgtttatttttttttgtttttgggtttttttatttattattagttcggataaggttcggtatttatgcgttagattattttggttttaaagcgttgattttgtaaaaaaagcggtttttttattattgtttcgtttatggttttcggcggtttttttcgcgttttttggttttgcggcgggaggcggggcgggaggcggggtcgggttttttggaggagttttcgttttttttcggttttcgtcggtagtcgttgggggggtcgcggtggggtgagggagaggagcgagaaggaatggtggagattttcggattagtcgggagttgtagtattttaggtttttattcgtcgtcgtgggggcgcgttagtggttttatcgaaagttttcggttttggcgcgagggttttggtttttgggcgtggtggttgcgcgtgacgttatatgtttaaggtgacgttagagtgaaagttatgatattatttcgaaagcgtttcgggtatcggagagatttatat

SEQ ID NO: 56 - DNA, 82 bp, Homo sapiens
cagggagaggtgggcagcgggctggagagcggcgccgggagttggagagcctgtgactttcccgtgaagttgctttgcacac

SEQ ID NO: 57 - DNA, 82 bp, Artificial Sequence
  converted
tagggagaggtgggtagcgggttggagagcggcgtcgggagttggagagtttgtgattttttcgtgaagttgttttgtatat

SEQ ID NO: 58 - DNA, 82 bp, Artificial Sequence
  converted
atatacaaaacaacttcacgaaaaaatcacaaactctccaactcccgacgccgctctccaacccgctacccacctctcccta

SEQ ID NO: 59 - DNA, 82 bp, Artificial Sequence
  converted
caaaaaaaaataaacaacgaactaaaaaacgacgccgaaaattaaaaaacctataactttcccgtaaaattactttacacac

SEQ ID NO: 60 - DNA, 82 bp, Artificial Sequence
  converted
gtgtgtaaagtaattttacgggaaagttataggttttttaattttcggcgtcgttttttagttcgttgtttatttttttttg

SEQ ID NO: 61 - DNA, 2615 bp, Homo sapiens
ccgggtcccggaccccgctgggcgccgcggaggcctccccctgctcctctgggtgcttggcctatccaaggccaagccagcggccgtggctgtgggactctggcctgcacactgtcccgaacccgctccgtgcctgggtgggagaaaccaccaacctcgctaggcctttcctcgccttcttccttagagccaagaccggagccgtcttgggctccgtagggggtgccgggaagggggagtgcttgcagctataatggttcctgggagccaacctttccgggctatctgcccgatcttgttttccccaacactacatttattttcttccgcggcccagcccgttccttgttttctgaagcattcggaagccagtgctccccaggctcccgccaacgactttccccaggacgaaattcattcgaaacgtggctctttacactagtatcaagaatgggccgaaagcacgatccggtttcaggaggtcggttaagagaaaaacacagtctcaccctagcctctccagccagaaaggatgagtgagcccccggctcctccggctccggtttcccagaccgcggaaccccagggcgaatactttcgatctttaaacacaggatggaaaacccttccccaggctaggcacccattcctcaaacagcttgggcctcaggacctgcgggaaagaataaggggacccgaccacgcacagcagacgcaattcgcgctcgggatcccgagtccctgcgcagtgcgggcactcgcgtccctcgcgcggtggagcgccaatcccaggtctgcggccagtcctatgctgggcattaatgaagtgtgcagagtctattaaagtggtttattcggggctaattgagcgtgagcaagttaactgcttgcattaatgagaacgggagcgaactccacgagtttgcgcctgggggagcccagcagcaacccaagaaaatcagccttgacatcgaatctccaacgagtggtgacaggcgtccggacccccgtgaagaggactgaccggcaccggatacttctatagcattctcccaacaaacgagatctaacgaacccattggcaaggcggtcatccggctgcacttaaatgtccgctgcgtcctcggtgatccattccccaatcttaataaaacagcaattacctcgaggagcctgggatggaacatctacacgccgccggtcgctgctagtcccctccagcgcttctcttccctagggggttgtgaacctgggacacctagccttgcacgtggttttgttccgcagagccaatgcgcagctcttagcctgggtgaaatttaccaaattgtggcaacaaagaaacccttgcggctactttacacattgagaacccaacccgctactgcctgagctgctgaaaaaggactaaacgtggtttttcattcttctccgagacatttccgaggagaaattagttcagcaggcagcccttcacccctttcccccttctttctctcctgacggctggattagcggacagtcaggggagtaacagaacttttcctgtccccagcccggagaccctagggctccacagagtttccactagtgctgtgtgtgggtctcgaattggaaagcagtgcttgcgcccactgcattgcctccctgcaccaggacaatcaagggttccgctccaggccttgacgacacagagcaatcatcctatggagaatatccctaagttagagcgcgagtgcaaggcggggttcagactcgcagccctgcgctctcgggtctaggcggcctcatactagagcgcaactcctcaaaagacaaacttgaacgaaagcgctaccgagctggggcatgcacctgtccctggcgcggtcggctgcggctgtggccattcactccctctcccttccttcttcgtcaacctgggcgtcagccagagctaggagcgcgtctcaggaaagtttgtgcccgctgaagttgctctggtttcttaaagggggcccacagattgactttcaaagtccgtgggcacctcgcccgtgattccgcagagccgggcgggctggccgcagtagcggaggcccgccccccttaatccccagcggtcagaggccgaggaccccgcgcaggaagtcctgaggcagcacccccaaccaccctgctctcactttcacaaaagtcctacagcattcgtttggcaagagcttccttcaggggcattgagagagaggaggcacccgccgagcagtgacaaggaacctgggagtcctgcccgcattcgctttgctgagcccaggcgcccaggactgcaattaccttgttccgcagaggtcctggggctgagcacgtctctggggctttagctgaaggggactggggaagccggaggccctggagcccacaggcgcccctggagctctagtgtcccgactcctctcctgccgcggggactccaagcgccggacacgcgggagcgagcgctcagcagccccggatcctgcacgccggggacggtgagcctcgctcgcggctccctcgctgggtctgaaccaggagcagctgagaacgtgccggggagaggccgagctccgactcactcatcccttagcaccgagccg

SEQ ID NO: 62 - DNA, 2615 bp, Artificial Sequence
  converted
tcgggtttcggatttcgttgggcgtcgcggaggtttttttttgtttttttgggtgtttggtttatttaaggttaagttagcggtcgtggttgtgggattttggtttgtatattgtttcgaattcgtttcgtgtttgggtgggagaaattattaatttcgttaggttttttttcgtttttttttttagagttaagatcggagtcgttttgggtttcgtagggggtgtcgggaagggggagtgtttgtagttataatggtttttgggagttaattttttcgggttatttgttcgattttgttttttttaatattatatttattttttttcgcggtttagttcgttttttgttttttgaagtattcggaagttagtgttttttaggttttcgttaacgattttttttaggacgaaatttattcgaaacgtggttttttatattagtattaagaatgggtcgaaagtacgattcggttttaggaggtcggttaagagaaaaatatagttttattttagtttttttagttagaaaggatgagtgagttttcggttttttcggtttcggttttttagatcgcggaattttagggcgaatattttcgatttttaaatataggatggaaaatttttttttaggttaggtatttattttttaaatagtttgggttttaggatttgcgggaaagaataaggggattcgattacgtatagtagacgtaattcgcgttcgggatttcgagtttttgcgtagtgcgggtattcgcgtttttcgcgcggtggagcgttaattttaggtttgcggttagttttatgttgggtattaatgaagtgtgtagagtttattaaagtggtttattcggggttaattgagcgtgagtaagttaattgtttgtattaatgagaacgggagcgaattttacgagtttgcgtttgggggagtttagtagtaatttaagaaaattagttttgatatcgaatttttaacgagtggtgataggcgttcggattttcgtgaagaggattgatcggtatcggatatttttatagtatttttttaataaacgagatttaacgaatttattggtaaggcggttattcggttgtatttaaatgttcgttgcgttttcggtgatttattttttaattttaataaaatagtaattatttcgaggagtttgggatggaatatttatacgtcgtcggtcgttgttagttttttttagcgttttttttttttagggggttgtgaatttgggatatttagttttgtacgtggttttgtttcgtagagttaatgcgtagtttttagtttgggtgaaatttattaaattgtggtaataaagaaatttttgcggttattttatatattgagaatttaattcgttattgtttgagttgttgaaaaaggattaaacgtggttttttattttttttcgagatattttcgaggagaaattagtttagtaggtagttttttatttttttttttttttttttttttttgacggttggattagcggatagttaggggagtaatagaattttttttgtttttagttcggagattttagggttttatagagtttttattagtgttgtgtgtgggtttcgaattggaaagtagtgtttgcgtttattgtattgtttttttgtattaggataattaagggtttcgttttaggttttgacgatatagagtaattattttatggagaatatttttaagttagagcgcgagtgtaaggcggggtttagattcgtagttttgcgttttcgggtttaggcggttttatattagagcgtaattttttaaaagataaatttgaacgaaagcgttatcgagttggggtatgtatttgtttttggcgcggtcggttgcggttgtggttatttattttttttttttttttttttcgttaatttgggcgttagttagagttaggagcgcgttttaggaaagtttgtgttcgttgaagttgttttggttttttaaagggggtttatagattgatttttaaagttcgtgggtatttcgttcgtgatttcgtagagtcgggcgggttggtcgtagtagcggaggttcgttttttttaatttttagcggttagaggtcgaggatttcgcgtaggaagttttgaggtagtatttttaattattttgtttttatttttataaaagttttatagtattcgtttggtaagagttttttttaggggtattgagagagaggaggtattcgtcgagtagtgataaggaatttgggagttttgttcgtattcgttttgttgagtttaggcgtttaggattgtaattattttgtttcgtagaggttttggggttgagtacgtttttggggttttagttgaaggggattggggaagtcggaggttttggagtttataggcgtttttggagttttagtgtttcgattttttttttgtcgcggggattttaagcgtcggatacgcgggagcgagcgtttagtagtttcggattttgtacgtcggggacggtgagtttcgttcgcggttttttcgttgggtttgaattaggagtagttgagaacgtgtcggggagaggtcgagtttcgatttatttattttttagtatcgagtcg

SEQ ID NO: 63 - DNA, 2615 bp, Artificial Sequence
  converted
cgactcgatactaaaaaataaataaatcgaaactcgacctctccccgacacgttctcaactactcctaattcaaacccaacgaaaaaaccgcgaacgaaactcaccgtccccgacgtacaaaatccgaaactactaaacgctcgctcccgcgtatccgacgcttaaaatccccgcgacaaaaaaaaaatcgaaacactaaaactccaaaaacgcctataaactccaaaacctccgacttccccaatccccttcaactaaaaccccaaaaacgtactcaaccccaaaacctctacgaaacaaaataattacaatcctaaacgcctaaactcaacaaaacgaatacgaacaaaactcccaaattccttatcactactcgacgaatacctcctctctctcaatacccctaaaaaaaactcttaccaaacgaatactataaaacttttataaaaataaaaacaaaataattaaaaatactacctcaaaacttcctacgcgaaatcctcgacctctaaccgctaaaaattaaaaaaaacgaacctccgctactacgaccaacccgcccgactctacgaaatcacgaacgaaatacccacgaactttaaaaatcaatctataaaccccctttaaaaaaccaaaacaacttcaacgaacacaaactttcctaaaacgcgctcctaactctaactaacgcccaaattaacgaaaaaaaaaaaaaaaaaaaataaataaccacaaccgcaaccgaccgcgccaaaaacaaatacataccccaactcgataacgctttcgttcaaatttatcttttaaaaaattacgctctaatataaaaccgcctaaacccgaaaacgcaaaactacgaatctaaaccccgccttacactcgcgctctaacttaaaaatattctccataaaataattactctatatcgtcaaaacctaaaacgaaacccttaattatcctaatacaaaaaaacaatacaataaacgcaaacactactttccaattcgaaacccacacacaacactaataaaaactctataaaaccctaaaatctccgaactaaaaacaaaaaaaattctattactcccctaactatccgctaatccaaccgtcaaaaaaaaaaaaaaaaaaaaaaaaataaaaaactacctactaaactaatttctcctcgaaaatatctcgaaaaaaaataaaaaaccacgtttaatcctttttcaacaactcaaacaataacgaattaaattctcaatatataaaataaccgcaaaaatttctttattaccacaatttaataaatttcacccaaactaaaaactacgcattaactctacgaaacaaaaccacgtacaaaactaaatatcccaaattcacaaccccctaaaaaaaaaaaacgctaaaaaaaactaacaacgaccgacgacgtataaatattccatcccaaactcctcgaaataattactattttattaaaattaaaaaataaatcaccgaaaacgcaacgaacatttaaatacaaccgaataaccgccttaccaataaattcgttaaatctcgtttattaaaaaaatactataaaaatatccgataccgatcaatcctcttcacgaaaatccgaacgcctatcaccactcgttaaaaattcgatatcaaaactaattttcttaaattactactaaactcccccaaacgcaaactcgtaaaattcgctcccgttctcattaatacaaacaattaacttactcacgctcaattaaccccgaataaaccactttaataaactctacacacttcattaatacccaacataaaactaaccgcaaacctaaaattaacgctccaccgcgcgaaaaacgcgaatacccgcactacgcaaaaactcgaaatcccgaacgcgaattacgtctactatacgtaatcgaatccccttattctttcccgcaaatcctaaaacccaaactatttaaaaaataaatacctaacctaaaaaaaaattttccatcctatatttaaaaatcgaaaatattcgccctaaaattccgcgatctaaaaaaccgaaaccgaaaaaaccgaaaactcactcatcctttctaactaaaaaaactaaaataaaactatatttttctcttaaccgacctcctaaaaccgaatcgtactttcgacccattcttaatactaatataaaaaaccacgtttcgaataaatttcgtcctaaaaaaaatcgttaacgaaaacctaaaaaacactaacttccgaatacttcaaaaaacaaaaaacgaactaaaccgcgaaaaaaaataaatataatattaaaaaaaacaaaatcgaacaaataacccgaaaaaattaactcccaaaaaccattataactacaaacactcccccttcccgacaccccctacgaaacccaaaacgactccgatcttaactctaaaaaaaaaaacgaaaaaaaacctaacgaaattaataatttctcccacccaaacacgaaacgaattcgaaacaatatacaaaccaaaatcccacaaccacgaccgctaacttaaccttaaataaaccaaacacccaaaaaaacaaaaaaaaacctccgcgacgcccaacgaaatccgaaacccga

SEQ ID NO: 64 - DNA, 2615 bp, Artificial Sequence
  converted
ccgaatcccgaaccccgctaaacgccgcgaaaacctccccctactcctctaaatacttaacctatccaaaaccaaaccaacgaccgtaactataaaactctaacctacacactatcccgaacccgctccgtacctaaataaaaaaaaccaccaacctcgctaaacctttcctcgccttcttccttaaaaccaaaaccgaaaccgtcttaaactccgtaaaaaataccgaaaaaaaaaaatacttacaactataataattcctaaaaaccaacctttccgaactatctacccgatcttattttccccaacactacatttattttcttccgcgacccaacccgttccttattttctaaaacattcgaaaaccaatactccccaaactcccgccaacgactttccccaaaacgaaattcattcgaaacgtaactctttacactaatatcaaaaataaaccgaaaacacgatccgatttcaaaaaatcgattaaaaaaaaaacacaatctcaccctaacctctccaaccaaaaaaaataaataaacccccgactcctccgactccgatttcccaaaccgcgaaaccccaaaacgaatactttcgatctttaaacacaaaataaaaaacccttccccaaactaaacacccattcctcaaacaacttaaacctcaaaacctacgaaaaaaaataaaaaaacccgaccacgcacaacaaacgcaattcgcgctcgaaatcccgaatccctacgcaatacgaacactcgcgtccctcgcgcgataaaacgccaatcccaaatctacgaccaatcctatactaaacattaataaaatatacaaaatctattaaaataatttattcgaaactaattaaacgtaaacaaattaactacttacattaataaaaacgaaaacgaactccacgaatttacgcctaaaaaaacccaacaacaacccaaaaaaatcaaccttaacatcgaatctccaacgaataataacaaacgtccgaacccccgtaaaaaaaactaaccgacaccgaatacttctataacattctcccaacaaacgaaatctaacgaacccattaacaaaacgatcatccgactacacttaaatatccgctacgtcctcgataatccattccccaatcttaataaaacaacaattacctcgaaaaacctaaaataaaacatctacacgccgccgatcgctactaatcccctccaacgcttctcttccctaaaaaattataaacctaaaacacctaaccttacacgtaattttattccgcaaaaccaatacgcaactcttaacctaaataaaatttaccaaattataacaacaaaaaaacccttacgactactttacacattaaaaacccaacccgctactacctaaactactaaaaaaaaactaaacgtaatttttcattcttctccgaaacatttccgaaaaaaaattaattcaacaaacaacccttcacccctttcccccttctttctctcctaacgactaaattaacgaacaatcaaaaaaataacaaaacttttcctatccccaacccgaaaaccctaaaactccacaaaatttccactaatactatatataaatctcgaattaaaaaacaatacttacgcccactacattacctccctacaccaaaacaatcaaaaattccgctccaaaccttaacgacacaaaacaatcatcctataaaaaatatccctaaattaaaacgcgaatacaaaacgaaattcaaactcgcaaccctacgctctcgaatctaaacgacctcatactaaaacgcaactcctcaaaaaacaaacttaaacgaaaacgctaccgaactaaaacatacacctatccctaacgcgatcgactacgactataaccattcactccctctcccttccttcttcgtcaacctaaacgtcaaccaaaactaaaaacgcgtctcaaaaaaatttatacccgctaaaattactctaatttcttaaaaaaaacccacaaattaactttcaaaatccgtaaacacctcgcccgtaattccgcaaaaccgaacgaactaaccgcaataacgaaaacccgccccccttaatccccaacgatcaaaaaccgaaaaccccgcgcaaaaaatcctaaaacaacacccccaaccaccctactctcactttcacaaaaatcctacaacattcgtttaacaaaaacttccttcaaaaacattaaaaaaaaaaaaacacccgccgaacaataacaaaaaacctaaaaatcctacccgcattcgctttactaaacccaaacgcccaaaactacaattaccttattccgcaaaaatcctaaaactaaacacgtctctaaaactttaactaaaaaaaactaaaaaaaccgaaaaccctaaaacccacaaacgcccctaaaactctaatatcccgactcctctcctaccgcgaaaactccaaacgccgaacacgcgaaaacgaacgctcaacaaccccgaatcctacacgccgaaaacgataaacctcgctcgcgactccctcgctaaatctaaaccaaaaacaactaaaaacgtaccgaaaaaaaaccgaactccgactcactcatcccttaacaccgaaccg

SEQ ID NO: 65 - DNA, 2615 bp, Artificial Sequence
  converted
cggttcggtgttaagggatgagtgagtcggagttcggttttttttcggtacgtttttagttgtttttggtttagatttagcgagggagtcgcgagcgaggtttatcgttttcggcgtgtaggattcggggttgttgagcgttcgttttcgcgtgttcggcgtttggagttttcgcggtaggagaggagtcgggatattagagttttaggggcgtttgtgggttttagggttttcggtttttttagttttttttagttaaagttttagagacgtgtttagttttaggatttttgcggaataaggtaattgtagttttgggcgtttgggtttagtaaagcgaatgcgggtaggatttttaggttttttgttattgttcggcgggtgtttttttttttttaatgtttttgaaggaagtttttgttaaacgaatgttgtaggatttttgtgaaagtgagagtagggtggttgggggtgttgttttaggattttttgcgcggggttttcggtttttgatcgttggggattaaggggggcgggttttcgttattgcggttagttcgttcggttttgcggaattacgggcgaggtgtttacggattttgaaagttaatttgtgggttttttttaagaaattagagtaattttagcgggtataaatttttttgagacgcgtttttagttttggttgacgtttaggttgacgaagaaggaagggagagggagtgaatggttatagtcgtagtcgatcgcgttagggataggtgtatgttttagttcggtagcgttttcgtttaagtttgttttttgaggagttgcgttttagtatgaggtcgtttagattcgagagcgtagggttgcgagtttgaatttcgttttgtattcgcgttttaatttagggatattttttataggatgattgttttgtgtcgttaaggtttggagcggaatttttgattgttttggtgtagggaggtaatgtagtgggcgtaagtattgttttttaattcgagatttatatatagtattagtggaaattttgtggagttttagggttttcgggttggggataggaaaagttttgttatttttttgattgttcgttaatttagtcgttaggagagaaagaagggggaaaggggtgaagggttgtttgttgaattaattttttttcggaaatgtttcggagaagaatgaaaaattacgtttagtttttttttagtagtttaggtagtagcgggttgggtttttaatgtgtaaagtagtcgtaagggtttttttgttgttataatttggtaaattttatttaggttaagagttgcgtattggttttgcggaataaaattacgtgtaaggttaggtgttttaggtttataattttttagggaagagaagcgttggaggggattagtagcgatcggcggcgtgtagatgttttattttaggtttttcgaggtaattgttgttttattaagattggggaatggattatcgaggacgtagcggatatttaagtgtagtcggatgatcgttttgttaatgggttcgttagatttcgtttgttgggagaatgttatagaagtattcggtgtcggttagttttttttacgggggttcggacgtttgttattattcgttggagattcgatgttaaggttgatttttttgggttgttgttgggtttttttaggcgtaaattcgtggagttcgttttcgtttttattaatgtaagtagttaatttgtttacgtttaattagtttcgaataaattattttaatagattttgtatattttattaatgtttagtataggattggtcgtagatttgggattggcgttttatcgcgcgagggacgcgagtgttcgtattgcgtagggattcgggatttcgagcgcgaattgcgtttgttgtgcgtggtcgggtttttttattttttttcgtaggttttgaggtttaagttgtttgaggaatgggtgtttagtttggggaagggttttttattttgtgtttaaagatcgaaagtattcgttttggggtttcgcggtttgggaaatcggagtcggaggagtcgggggtttatttattttttttggttggagaggttagggtgagattgtgttttttttttaatcgattttttgaaatcggatcgtgttttcggtttatttttgatattagtgtaaagagttacgtttcgaatgaatttcgttttggggaaagtcgttggcgggagtttggggagtattggttttcgaatgttttagaaaataaggaacgggttgggtcgcggaagaaaataaatgtagtgttggggaaaataagatcgggtagatagttcggaaaggttggtttttaggaattattatagttgtaagtatttttttttttcggtattttttacggagtttaagacggtttcggttttggttttaaggaagaaggcgaggaaaggtttagcgaggttggtggttttttttatttaggtacggagcgggttcgggatagtgtgtaggttagagttttatagttacggtcgttggtttggttttggataggttaagtatttagaggagtagggggaggttttcgcggcgtttagcggggttcgggattcgg

SEQ ID NO: 66 - DNA, 1095 bp, Homo sapiens
cattcctcaaacagcttgggcctcaggacctgcgggaaagaataaggggacccgaccacgcacagcagacgcaattcgcgctcgggatcccgagtccctgcgcagtgcgggcactcgcgtccctcgcgcggtggagcgccaatcccaggtctgcggccagtcctatgctgggcattaatgaagtgtgcagagtctattaaagtggtttattcggggctaattgagcgtgagcaagttaactgcttgcattaatgagaacgggagcgaactccacgagtttgcgcctgggggagcccagcagcaacccaagaaaatcagccttgacatcgaatctccaacgagtggtgacaggcgtccggacccccgtgaagaggactgaccggcaccggatacttctatagcattctcccaacaaacgagatctaacgaacccattggcaaggcggtcatccggctgcacttaaatgtccgctgcgtcctcggtgatccattccccaatcttaataaaacagcaattacctcgaggagcctgggatggaacatctacacgccgccggtcgctgctagtcccctccagcgcttctcttccctagggggttgtgaacctgggacacctagccttgcacgtggttttgttccgcagagccaatgcgcagctcttagcctgggtgaaatttaccaaattgtggcaacaaagaaacccttgcggctactttacacattgagaacccaacccgctactgcctgagctgctgaaaaaggactaaacgtggtttttcattcttctccgagacatttccgaggagaaattagttcagcaggcagcccttcacccctttcccccttctttctctcctgacggctggattagcggacagtcaggggagtaacagaacttttcctgtccccagcccggagaccctagggctccacagagtttccactagtgctgtgtgtgggtctcgaattggaaagcagtgcttgcgcccactgcattgcctccctgcaccaggacaatcaagggttccgctccaggccttgacgacacagagcaatcatcctatggagaatatccctaagttagagcgcgagtgc

SEQ ID NO: 67 - DNA, 1095 bp, Artificial Sequence
  converted
tattttttaaatagtttgggttttaggatttgcgggaaagaataaggggattcgattacgtatagtagacgtaattcgcgttcgggatttcgagtttttgcgtagtgcgggtattcgcgtttttcgcgcggtggagcgttaattttaggtttgcggttagttttatgttgggtattaatgaagtgtgtagagtttattaaagtggtttattcggggttaattgagcgtgagtaagttaattgtttgtattaatgagaacgggagcgaattttacgagtttgcgtttgggggagtttagtagtaatttaagaaaattagttttgatatcgaatttttaacgagtggtgataggcgttcggattttcgtgaagaggattgatcggtatcggatatttttatagtatttttttaataaacgagatttaacgaatttattggtaaggcggttattcggttgtatttaaatgttcgttgcgttttcggtgatttattttttaattttaataaaatagtaattatttcgaggagtttgggatggaatatttatacgtcgtcggtcgttgttagttttttttagcgttttttttttttagggggttgtgaatttgggatatttagttttgtacgtggttttgtttcgtagagttaatgcgtagtttttagtttgggtgaaatttattaaattgtggtaataaagaaatttttgcggttattttatatattgagaatttaattcgttattgtttgagttgttgaaaaaggattaaacgtggttttttattttttttcgagatattttcgaggagaaattagtttagtaggtagttttttatttttttttttttttttttttttttgacggttggattagcggatagttaggggagtaatagaattttttttgtttttagttcggagattttagggttttatagagtttttattagtgttgtgtgtgggtttcgaattggaaagtagtgtttgcgtttattgtattgtttttttgtattaggataattaagggtttcgttttaggttttgacgatatagagtaattattttatggagaatatttttaagttagagcgcgagtgt

SEQ ID NO: 68 - DNA, 1095 bp, Artificial Sequence
  converted
acactcgcgctctaacttaaaaatattctccataaaataattactctatatcgtcaaaacctaaaacgaaacccttaattatcctaatacaaaaaaacaatacaataaacgcaaacactactttccaattcgaaacccacacacaacactaataaaaactctataaaaccctaaaatctccgaactaaaaacaaaaaaaattctattactcccctaactatccgctaatccaaccgtcaaaaaaaaaaaaaaaaaaaaaaaaataaaaaactacctactaaactaatttctcctcgaaaatatctcgaaaaaaaataaaaaaccacgtttaatcctttttcaacaactcaaacaataacgaattaaattctcaatatataaaataaccgcaaaaatttctttattaccacaatttaataaatttcacccaaactaaaaactacgcattaactctacgaaacaaaaccacgtacaaaactaaatatcccaaattcacaaccccctaaaaaaaaaaaacgctaaaaaaaactaacaacgaccgacgacgtataaatattccatcccaaactcctcgaaataattactattttattaaaattaaaaaataaatcaccgaaaacgcaacgaacatttaaatacaaccgaataaccgccttaccaataaattcgttaaatctcgtttattaaaaaaatactataaaaatatccgataccgatcaatcctcttcacgaaaatccgaacgcctatcaccactcgttaaaaattcgatatcaaaactaattttcttaaattactactaaactcccccaaacgcaaactcgtaaaattcgctcccgttctcattaatacaaacaattaacttactcacgctcaattaaccccgaataaaccactttaataaactctacacacttcattaatacccaacataaaactaaccgcaaacctaaaattaacgctccaccgcgcgaaaaacgcgaatacccgcactacgcaaaaactcgaaatcccgaacgcgaattacgtctactatacgtaatcgaatccccttattctttcccgcaaatcctaaaacccaaactatttaaaaaata

SEQ ID NO: 69 - DNA, 1095 bp, Artificial Sequence
  converted
cattcctcaaacaacttaaacctcaaaacctacgaaaaaaaataaaaaaacccgaccacgcacaacaaacgcaattcgcgctcgaaatcccgaatccctacgcaatacgaacactcgcgtccctcgcgcgataaaacgccaatcccaaatctacgaccaatcctatactaaacattaataaaatatacaaaatctattaaaataatttattcgaaactaattaaacgtaaacaaattaactacttacattaataaaaacgaaaacgaactccacgaatttacgcctaaaaaaacccaacaacaacccaaaaaaatcaaccttaacatcgaatctccaacgaataataacaaacgtccgaacccccgtaaaaaaaactaaccgacaccgaatacttctataacattctcccaacaaacgaaatctaacgaacccattaacaaaacgatcatccgactacacttaaatatccgctacgtcctcgataatccattccccaatcttaataaaacaacaattacctcgaaaaacctaaaataaaacatctacacgccgccgatcgctactaatcccctccaacgcttctcttccctaaaaaattataaacctaaaacacctaaccttacacgtaattttattccgcaaaaccaatacgcaactcttaacctaaataaaatttaccaaattataacaacaaaaaaacccttacgactactttacacattaaaaacccaacccgctactacctaaactactaaaaaaaaactaaacgtaatttttcattcttctccgaaacatttccgaaaaaaaattaattcaacaaacaacccttcacccctttcccccttctttctctcctaacgactaaattaacgaacaatcaaaaaaataacaaaacttttcctatccccaacccgaaaaccctaaaactccacaaaatttccactaatactatatataaatctcgaattaaaaaacaatacttacgcccactacattacctccctacaccaaaacaatcaaaaattccgctccaaaccttaacgacacaaaacaatcatcctataaaaaatatccctaaattaaaacgcgaatac

SEQ ID NO: 70 - DNA, 1095 bp, Artificial Sequence
  converted
gtattcgcgttttaatttagggatattttttataggatgattgttttgtgtcgttaaggtttggagcggaatttttgattgttttggtgtagggaggtaatgtagtgggcgtaagtattgttttttaattcgagatttatatatagtattagtggaaattttgtggagttttagggttttcgggttggggataggaaaagttttgttatttttttgattgttcgttaatttagtcgttaggagagaaagaagggggaaaggggtgaagggttgtttgttgaattaattttttttcggaaatgtttcggagaagaatgaaaaattacgtttagtttttttttagtagtttaggtagtagcgggttgggtttttaatgtgtaaagtagtcgtaagggtttttttgttgttataatttggtaaattttatttaggttaagagttgcgtattggttttgcggaataaaattacgtgtaaggttaggtgttttaggtttataattttttagggaagagaagcgttggaggggattagtagcgatcggcggcgtgtagatgttttattttaggtttttcgaggtaattgttgttttattaagattggggaatggattatcgaggacgtagcggatatttaagtgtagtcggatgatcgttttgttaatgggttcgttagatttcgtttgttgggagaatgttatagaagtattcggtgtcggttagttttttttacgggggttcggacgtttgttattattcgttggagattcgatgttaaggttgatttttttgggttgttgttgggtttttttaggcgtaaattcgtggagttcgttttcgtttttattaatgtaagtagttaatttgtttacgtttaattagtttcgaataaattattttaatagattttgtatattttattaatgtttagtataggattggtcgtagatttgggattggcgttttatcgcgcgagggacgcgagtgttcgtattgcgtagggattcgggatttcgagcgcgaattgcgtttgttgtgcgtggtcgggtttttttattttttttcgtaggttttgaggtttaagttgtttgaggaatg

SEQ ID NO: 71 - DNA, 95 bp, Homo sapiens
ttaataaaacagcaattacctcgaggagcctgggatggaacatctacacgccgccggtcgctgctagtcccctccagcgcttctcttccctaggg

SEQ ID NO: 72 - DNA, 95 bp, Artificial Sequence
  converted
ttaataaaatagtaattatttcgaggagtttgggatggaatatttatacgtcgtcggtcgttgttagttttttttagcgttttttttttttaggg

SEQ ID NO: 73 - DNA, 95 bp, Artificial Sequence
  converted
ccctaaaaaaaaaaaacgctaaaaaaaactaacaacgaccgacgacgtataaatattccatcccaaactcctcgaaataattactattttattaa

SEQ ID NO: 74 - DNA, 95 bp, Artificial Sequence
  converted
ttaataaaacaacaattacctcgaaaaacctaaaataaaacatctacacgccgccgatcgctactaatcccctccaacgcttctcttccctaaaa

SEQ ID NO: 75 - DNA, 95 bp, Artificial Sequence
  converted
ttttagggaagagaagcgttggaggggattagtagcgatcggcggcgtgtagatgttttattttaggtttttcgaggtaattgttgttttattaa

SEQ ID NO: 76 - DNA, 2110 bp, Homo sapiens
acgcctaggtatgcgcgcccatgagccggcactcacttgagtccctgcacatgcccacctgggccatacaggcgccagtagtacccctcgggggacacctgtattctcgcgtgcacacctgggttccacgtacatctggatctctgcgcgctcccacctgtgacccactcagctcacgacccgctcggttcccgcccgcccccgcccccgcccccgccgccgccgccgccgccgccgccgccaccgcccgggtgtacctggatgttgagcttgcgctcacaggccggtcccgtgccctgcaccacgctgtcgagcaccgccaccacgtcgggcgccggttccacgaagcaggcggtgcgcgcagctcggatggcggctcgcacgtcggcgaagcggaaggcgcagagtgcggccggagcagcgcgggccgcgggggacccctggggccgctcgaagacagcaaagagccgctcccgggctgggaagaccgacaccaggcggctgtagaggtcgccgcggcccgcgccgcccgcgcactgcaagcccaactggatgtaggactcggtgagcttcttggcgtcgccgccggcgccgtggggcaggcagatgcgcgccagcaggctccgcgcctggctctccttgtcgcccgcgcgcgcctcgctgttgagcgccaggtacgcgtaggactgtgcacccggcggcgggtcggacgggtgcaggaaggcgctcacgaagcccagcttgtgctgctccttggcgccctgcttgatcttgaggatgttgtcgtcggaggggttgaggtcgaaggtgaagagcttggccaggtcgccgcgcgtgtccagggagcggatggcgatctcgggcgtgttctcgaagcggtggtcctccaggctgcggttgcgcgggaagaaggagctgccgtaaccggtgtacgtggcgcccacgagcaggcggctgccccccgcgcccgcggcgggaggcagaactagccccacggtggacgcgttcgggtggttggccgccacgttcagcatgctggggaacaccgtgacgggctcggcgggcggcgcggcgggcgggaagcgcacggccacggccgagatgttgccccggcgccgcagctggcagaagccctggtagatggacccgcacacgactaccaggccctggccggggtccagctgcaggatcttgttgtagttgtccgtgaggcgccgcgggtgctcgcacgaggcctgcggcagctgcggagcgtgacacagcgggctgtcgggcaccgggcccacggccgcctcggcctccaggctcaggttggcgcccgacagctgatagaggcggttgacggccgccaggtacacggtccccgccgcgccgtccagggcgaagttgttggtgggcgtgggcgaggggaaccgacgctggatctccagggcgccggcccgcgccgcccccaggagcagcagcaacagcagcggcaccgggcaccgcggcggcgtctggaacggcggggggctggcggcggcggcccgggcgctaaggggtgcgccgcccgcggcgcgaggagccatccgggcgtgcgcgggctgcgcggcgcggcgagtgcatggggcgaggcgcggccgggagccgggagcccggaggcggcgggaggcggggggcgggcccgggccgcgatgcgcttcctgcccgcgccagccgcccccgaccccggcgcccgggcccgggtcagcctcgccgcgcagcccgggggcggggcgggggcggctccgctgcggacacgccctcactccccgccccggccccgcccgcccccgggacccccgcctgcccctggccgccctgggccacgccccgccgtccgcgggtcccgccgcccgcgccctccaggacccgcccgcggccccagggtccctccccgtagccgccgccgccgtcgctcgctctcctcgctctttcctcccacttggccgcttggcctgcctcgctcgcgcctgttttctcttttccgccctctcccccaccccgtctctccctctctctgcgtctctcggtctctgcgtctctccgtctctgtccccaccccaaactccgcgcgtgtggcgcttctccg

SEQ ID NO: 77 - DNA, 2110 bp, Artificial Sequence
  converted
acgtttaggtatgcgcgtttatgagtcggtatttatttgagtttttgtatatgtttatttgggttatataggcgttagtagtatttttcgggggatatttgtattttcgcgtgtatatttgggttttacgtatatttggatttttgcgcgtttttatttgtgatttatttagtttacgattcgttcggttttcgttcgttttcgttttcgttttcgtcgtcgtcgtcgtcgtcgtcgtcgttatcgttcgggtgtatttggatgttgagtttgcgtttataggtcggtttcgtgttttgtattacgttgtcgagtatcgttattacgtcgggcgtcggttttacgaagtaggcggtgcgcgtagttcggatggcggttcgtacgtcggcgaagcggaaggcgtagagtgcggtcggagtagcgcgggtcgcgggggatttttggggtcgttcgaagatagtaaagagtcgttttcgggttgggaagatcgatattaggcggttgtagaggtcgtcgcggttcgcgtcgttcgcgtattgtaagtttaattggatgtaggattcggtgagttttttggcgtcgtcgtcggcgtcgtggggtaggtagatgcgcgttagtaggtttcgcgtttggttttttttgtcgttcgcgcgcgtttcgttgttgagcgttaggtacgcgtaggattgtgtattcggcggcgggtcggacgggtgtaggaaggcgtttacgaagtttagtttgtgttgttttttggcgttttgtttgattttgaggatgttgtcgtcggaggggttgaggtcgaaggtgaagagtttggttaggtcgtcgcgcgtgtttagggagcggatggcgatttcgggcgtgttttcgaagcggtggttttttaggttgcggttgcgcgggaagaaggagttgtcgtaatcggtgtacgtggcgtttacgagtaggcggttgtttttcgcgttcgcggcgggaggtagaattagttttacggtggacgcgttcgggtggttggtcgttacgtttagtatgttggggaatatcgtgacgggttcggcgggcggcgcggcgggcgggaagcgtacggttacggtcgagatgttgtttcggcgtcgtagttggtagaagttttggtagatggattcgtatacgattattaggttttggtcggggtttagttgtaggattttgttgtagttgttcgtgaggcgtcgcgggtgttcgtacgaggtttgcggtagttgcggagcgtgatatagcgggttgtcgggtatcgggtttacggtcgtttcggtttttaggtttaggttggcgttcgatagttgatagaggcggttgacggtcgttaggtatacggttttcgtcgcgtcgtttagggcgaagttgttggtgggcgtgggcgaggggaatcgacgttggatttttagggcgtcggttcgcgtcgtttttaggagtagtagtaatagtagcggtatcgggtatcgcggcggcgtttggaacggcggggggttggcggcggcggttcgggcgttaaggggtgcgtcgttcgcggcgcgaggagttattcgggcgtgcgcgggttgcgcggcgcggcgagtgtatggggcgaggcgcggtcgggagtcgggagttcggaggcggcgggaggcggggggcgggttcgggtcgcgatgcgttttttgttcgcgttagtcgttttcgatttcggcgttcgggttcgggttagtttcgtcgcgtagttcgggggcggggcgggggcggtttcgttgcggatacgtttttatttttcgtttcggtttcgttcgttttcgggattttcgtttgtttttggtcgttttgggttacgtttcgtcgttcgcgggtttcgtcgttcgcgttttttaggattcgttcgcggttttagggttttttttcgtagtcgtcgtcgtcgtcgttcgtttttttcgttttttttttttatttggtcgtttggtttgtttcgttcgcgtttgttttttttttttcgtttttttttttatttcgtttttttttttttttgcgtttttcggtttttgcgttttttcgtttttgtttttattttaaatttcgcgcgtgtggcgttttttcg

SEQ ID NO: 78 - DNA, 2110 bp, Artificial Sequence
  converted
cgaaaaaacgccacacgcgcgaaatttaaaataaaaacaaaaacgaaaaaacgcaaaaaccgaaaaacgcaaaaaaaaaaaaaaacgaaataaaaaaaaaaacgaaaaaaaaaaaacaaacgcgaacgaaacaaaccaaacgaccaaataaaaaaaaaaaacgaaaaaaacgaacgacgacgacgacgactacgaaaaaaaaccctaaaaccgcgaacgaatcctaaaaaacgcgaacgacgaaacccgcgaacgacgaaacgtaacccaaaacgaccaaaaacaaacgaaaatcccgaaaacgaacgaaaccgaaacgaaaaataaaaacgtatccgcaacgaaaccgcccccgccccgcccccgaactacgcgacgaaactaacccgaacccgaacgccgaaatcgaaaacgactaacgcgaacaaaaaacgcatcgcgacccgaacccgccccccgcctcccgccgcctccgaactcccgactcccgaccgcgcctcgccccatacactcgccgcgccgcgcaacccgcgcacgcccgaataactcctcgcgccgcgaacgacgcaccccttaacgcccgaaccgccgccgccaaccccccgccgttccaaacgccgccgcgatacccgataccgctactattactactactcctaaaaacgacgcgaaccgacgccctaaaaatccaacgtcgattcccctcgcccacgcccaccaacaacttcgccctaaacgacgcgacgaaaaccgtatacctaacgaccgtcaaccgcctctatcaactatcgaacgccaacctaaacctaaaaaccgaaacgaccgtaaacccgatacccgacaacccgctatatcacgctccgcaactaccgcaaacctcgtacgaacacccgcgacgcctcacgaacaactacaacaaaatcctacaactaaaccccgaccaaaacctaataatcgtatacgaatccatctaccaaaacttctaccaactacgacgccgaaacaacatctcgaccgtaaccgtacgcttcccgcccgccgcgccgcccgccgaacccgtcacgatattccccaacatactaaacgtaacgaccaaccacccgaacgcgtccaccgtaaaactaattctacctcccgccgcgaacgcgaaaaacaaccgcctactcgtaaacgccacgtacaccgattacgacaactccttcttcccgcgcaaccgcaacctaaaaaaccaccgcttcgaaaacacgcccgaaatcgccatccgctccctaaacacgcgcgacgacctaaccaaactcttcaccttcgacctcaacccctccgacgacaacatcctcaaaatcaaacaaaacgccaaaaaacaacacaaactaaacttcgtaaacgccttcctacacccgtccgacccgccgccgaatacacaatcctacgcgtacctaacgctcaacaacgaaacgcgcgcgaacgacaaaaaaaaccaaacgcgaaacctactaacgcgcatctacctaccccacgacgccgacgacgacgccaaaaaactcaccgaatcctacatccaattaaacttacaatacgcgaacgacgcgaaccgcgacgacctctacaaccgcctaatatcgatcttcccaacccgaaaacgactctttactatcttcgaacgaccccaaaaatcccccgcgacccgcgctactccgaccgcactctacgccttccgcttcgccgacgtacgaaccgccatccgaactacgcgcaccgcctacttcgtaaaaccgacgcccgacgtaataacgatactcgacaacgtaatacaaaacacgaaaccgacctataaacgcaaactcaacatccaaatacacccgaacgataacgacgacgacgacgacgacgacgacgaaaacgaaaacgaaaacgaacgaaaaccgaacgaatcgtaaactaaataaatcacaaataaaaacgcgcaaaaatccaaatatacgtaaaacccaaatatacacgcgaaaatacaaatatcccccgaaaaatactactaacgcctatataacccaaataaacatatacaaaaactcaaataaataccgactcataaacgcgcatacctaaacgt

SEQ ID NO: 79 - DNA, 2110 bp, Artificial Sequence
  converted
acgcctaaatatacgcgcccataaaccgacactcacttaaatccctacacatacccacctaaaccatacaaacgccaataatacccctcgaaaaacacctatattctcgcgtacacacctaaattccacgtacatctaaatctctacgcgctcccacctataacccactcaactcacgacccgctcgattcccgcccgcccccgcccccgcccccgccgccgccgccgccgccgccgccgccaccgcccgaatatacctaaatattaaacttacgctcacaaaccgatcccgtaccctacaccacgctatcgaacaccgccaccacgtcgaacgccgattccacgaaacaaacgatacgcgcaactcgaataacgactcgcacgtcgacgaaacgaaaaacgcaaaatacgaccgaaacaacgcgaaccgcgaaaaacccctaaaaccgctcgaaaacaacaaaaaaccgctcccgaactaaaaaaaccgacaccaaacgactataaaaatcgccgcgacccgcgccgcccgcgcactacaaacccaactaaatataaaactcgataaacttcttaacgtcgccgccgacgccgtaaaacaaacaaatacgcgccaacaaactccgcgcctaactctccttatcgcccgcgcgcgcctcgctattaaacgccaaatacgcgtaaaactatacacccgacgacgaatcgaacgaatacaaaaaaacgctcacgaaacccaacttatactactccttaacgccctacttaatcttaaaaatattatcgtcgaaaaaattaaaatcgaaaataaaaaacttaaccaaatcgccgcgcgtatccaaaaaacgaataacgatctcgaacgtattctcgaaacgataatcctccaaactacgattacgcgaaaaaaaaaaactaccgtaaccgatatacgtaacgcccacgaacaaacgactaccccccgcgcccgcgacgaaaaacaaaactaaccccacgataaacgcgttcgaataattaaccgccacgttcaacatactaaaaaacaccgtaacgaactcgacgaacgacgcgacgaacgaaaaacgcacgaccacgaccgaaatattaccccgacgccgcaactaacaaaaaccctaataaataaacccgcacacgactaccaaaccctaaccgaaatccaactacaaaatcttattataattatccgtaaaacgccgcgaatactcgcacgaaacctacgacaactacgaaacgtaacacaacgaactatcgaacaccgaacccacgaccgcctcgacctccaaactcaaattaacgcccgacaactaataaaaacgattaacgaccgccaaatacacgatccccgccgcgccgtccaaaacgaaattattaataaacgtaaacgaaaaaaaccgacgctaaatctccaaaacgccgacccgcgccgcccccaaaaacaacaacaacaacaacgacaccgaacaccgcgacgacgtctaaaacgacgaaaaactaacgacgacgacccgaacgctaaaaaatacgccgcccgcgacgcgaaaaaccatccgaacgtacgcgaactacgcgacgcgacgaatacataaaacgaaacgcgaccgaaaaccgaaaacccgaaaacgacgaaaaacgaaaaacgaacccgaaccgcgatacgcttcctacccgcgccaaccgcccccgaccccgacgcccgaacccgaatcaacctcgccgcgcaacccgaaaacgaaacgaaaacgactccgctacgaacacgccctcactccccgccccgaccccgcccgcccccgaaacccccgcctacccctaaccgccctaaaccacgccccgccgtccgcgaatcccgccgcccgcgccctccaaaacccgcccgcgaccccaaaatccctccccgtaaccgccgccgccgtcgctcgctctcctcgctctttcctcccacttaaccgcttaacctacctcgctcgcgcctattttctcttttccgccctctcccccaccccgtctctccctctctctacgtctctcgatctctacgtctctccgtctctatccccaccccaaactccgcgcgtataacgcttctccg

SEQ ID NO: 80 - DNA, 2110 bp, Artificial Sequence
  converted
cggagaagcgttatacgcgcggagtttggggtggggatagagacggagagacgtagagatcgagagacgtagagagagggagagacggggtgggggagagggcggaaaagagaaaataggcgcgagcgaggtaggttaagcggttaagtgggaggaaagagcgaggagagcgagcgacggcggcggcggttacggggagggattttggggtcgcgggcgggttttggagggcgcgggcggcgggattcgcggacggcggggcgtggtttagggcggttaggggtaggcgggggtttcgggggcgggcggggtcggggcggggagtgagggcgtgttcgtagcggagtcgttttcgtttcgttttcgggttgcgcggcgaggttgattcgggttcgggcgtcggggtcgggggcggttggcgcgggtaggaagcgtatcgcggttcgggttcgtttttcgtttttcgtcgttttcgggttttcggttttcggtcgcgtttcgttttatgtattcgtcgcgtcgcgtagttcgcgtacgttcggatggtttttcgcgtcgcgggcggcgtattttttagcgttcgggtcgtcgtcgttagtttttcgtcgttttagacgtcgtcgcggtgttcggtgtcgttgttgttgttgttgtttttgggggcggcgcgggtcggcgttttggagatttagcgtcggtttttttcgtttacgtttattaataatttcgttttggacggcgcggcggggatcgtgtatttggcggtcgttaatcgtttttattagttgtcgggcgttaatttgagtttggaggtcgaggcggtcgtgggttcggtgttcgatagttcgttgtgttacgtttcgtagttgtcgtaggtttcgtgcgagtattcgcggcgttttacggataattataataagattttgtagttggatttcggttagggtttggtagtcgtgtgcgggtttatttattagggtttttgttagttgcggcgtcggggtaatatttcggtcgtggtcgtgcgtttttcgttcgtcgcgtcgttcgtcgagttcgttacggtgttttttagtatgttgaacgtggcggttaattattcgaacgcgtttatcgtggggttagttttgtttttcgtcgcgggcgcggggggtagtcgtttgttcgtgggcgttacgtatatcggttacggtagtttttttttttcgcgtaatcgtagtttggaggattatcgtttcgagaatacgttcgagatcgttattcgttttttggatacgcgcggcgatttggttaagttttttattttcgattttaattttttcgacgataatatttttaagattaagtagggcgttaaggagtagtataagttgggtttcgtgagcgtttttttgtattcgttcgattcgtcgtcgggtgtatagttttacgcgtatttggcgtttaatagcgaggcgcgcgcgggcgataaggagagttaggcgcggagtttgttggcgcgtatttgtttgttttacggcgtcggcggcgacgttaagaagtttatcgagttttatatttagttgggtttgtagtgcgcgggcggcgcgggtcgcggcgatttttatagtcgtttggtgtcggtttttttagttcgggagcggttttttgttgttttcgagcggttttaggggtttttcgcggttcgcgttgtttcggtcgtattttgcgtttttcgtttcgtcgacgtgcgagtcgttattcgagttgcgcgtatcgtttgtttcgtggaatcggcgttcgacgtggtggcggtgttcgatagcgtggtgtagggtacgggatcggtttgtgagcgtaagtttaatatttaggtatattcgggcggtggcggcggcggcggcggcggcggcggcgggggcgggggcgggggcgggcgggaatcgagcgggtcgtgagttgagtgggttataggtgggagcgcgtagagatttagatgtacgtggaatttaggtgtgtacgcgagaatataggtgtttttcgaggggtattattggcgtttgtatggtttaggtgggtatgtgtagggatttaagtgagtgtcggtttatgggcgcgtatatttaggcgt

SEQ ID NO: 81 - DNA, 1085 bp, Homo sapiens
tcgagcaccgccaccacgtcgggcgccggttccacgaagcaggcggtgcgcgcagctcggatggcggctcgcacgtcggcgaagcggaaggcgcagagtgcggccggagcagcgcgggccgcgggggacccctggggccgctcgaagacagcaaagagccgctcccgggctgggaagaccgacaccaggcggctgtagaggtcgccgcggcccgcgccgcccgcgcactgcaagcccaactggatgtaggactcggtgagcttcttggcgtcgccgccggcgccgtggggcaggcagatgcgcgccagcaggctccgcgcctggctctccttgtcgcccgcgcgcgcctcgctgttgagcgccaggtacgcgtaggactgtgcacccggcggcgggtcggacgggtgcaggaaggcgctcacgaagcccagcttgtgctgctccttggcgccctgcttgatcttgaggatgttgtcgtcggaggggttgaggtcgaaggtgaagagcttggccaggtcgccgcgcgtgtccagggagcggatggcgatctcgggcgtgttctcgaagcggtggtcctccaggctgcggttgcgcgggaagaaggagctgccgtaaccggtgtacgtggcgcccacgagcaggcggctgccccccgcgcccgcggcgggaggcagaactagccccacggtggacgcgttcgggtggttggccgccacgttcagcatgctggggaacaccgtgacgggctcggcgggcggcgcggcgggcgggaagcgcacggccacggccgagatgttgccccggcgccgcagctggcagaagccctggtagatggacccgcacacgactaccaggccctggccggggtccagctgcaggatcttgttgtagttgtccgtgaggcgccgcgggtgctcgcacgaggcctgcggcagctgcggagcgtgacacagcgggctgtcgggcaccgggcccacggccgcctcggcctccaggctcaggttggcgcccgacagctgatagaggcggttgacggccgccaggtacacggtccccgccgcgccgtccagggcgaagttgttggt

SEQ ID NO: 82 - DNA, 1085 bp, Artificial Sequence
  converted
tcgagtatcgttattacgtcgggcgtcggttttacgaagtaggcggtgcgcgtagttcggatggcggttcgtacgtcggcgaagcggaaggcgtagagtgcggtcggagtagcgcgggtcgcgggggatttttggggtcgttcgaagatagtaaagagtcgttttcgggttgggaagatcgatattaggcggttgtagaggtcgtcgcggttcgcgtcgttcgcgtattgtaagtttaattggatgtaggattcggtgagttttttggcgtcgtcgtcggcgtcgtggggtaggtagatgcgcgttagtaggtttcgcgtttggttttttttgtcgttcgcgcgcgtttcgttgttgagcgttaggtacgcgtaggattgtgtattcggcggcgggtcggacgggtgtaggaaggcgtttacgaagtttagtttgtgttgttttttggcgttttgtttgattttgaggatgttgtcgtcggaggggttgaggtcgaaggtgaagagtttggttaggtcgtcgcgcgtgtttagggagcggatggcgatttcgggcgtgttttcgaagcggtggttttttaggttgcggttgcgcgggaagaaggagttgtcgtaatcggtgtacgtggcgtttacgagtaggcggttgtttttcgcgttcgcggcgggaggtagaattagttttacggtggacgcgttcgggtggttggtcgttacgtttagtatgttggggaatatcgtgacgggttcggcgggcggcgcggcgggcgggaagcgtacggttacggtcgagatgttgtttcggcgtcgtagttggtagaagttttggtagatggattcgtatacgattattaggttttggtcggggtttagttgtaggattttgttgtagttgttcgtgaggcgtcgcgggtgttcgtacgaggtttgcggtagttgcggagcgtgatatagcgggttgtcgggtatcgggtttacggtcgtttcggtttttaggtttaggttggcgttcgatagttgatagaggcggttgacggtcgttaggtatacggttttcgtcgcgtcgtttagggcgaagttgttggt

SEQ ID NO: 83 - DNA, 1085 bp, Artificial Sequence
  converted
accaacaacttcgccctaaacgacgcgacgaaaaccgtatacctaacgaccgtcaaccgcctctatcaactatcgaacgccaacctaaacctaaaaaccgaaacgaccgtaaacccgatacccgacaacccgctatatcacgctccgcaactaccgcaaacctcgtacgaacacccgcgacgcctcacgaacaactacaacaaaatcctacaactaaaccccgaccaaaacctaataatcgtatacgaatccatctaccaaaacttctaccaactacgacgccgaaacaacatctcgaccgtaaccgtacgcttcccgcccgccgcgccgcccgccgaacccgtcacgatattccccaacatactaaacgtaacgaccaaccacccgaacgcgtccaccgtaaaactaattctacctcccgccgcgaacgcgaaaaacaaccgcctactcgtaaacgccacgtacaccgattacgacaactccttcttcccgcgcaaccgcaacctaaaaaaccaccgcttcgaaaacacgcccgaaatcgccatccgctccctaaacacgcgcgacgacctaaccaaactcttcaccttcgacctcaacccctccgacgacaacatcctcaaaatcaaacaaaacgccaaaaaacaacacaaactaaacttcgtaaacgccttcctacacccgtccgacccgccgccgaatacacaatcctacgcgtacctaacgctcaacaacgaaacgcgcgcgaacgacaaaaaaaaccaaacgcgaaacctactaacgcgcatctacctaccccacgacgccgacgacgacgccaaaaaactcaccgaatcctacatccaattaaacttacaatacgcgaacgacgcgaaccgcgacgacctctacaaccgcctaatatcgatcttcccaacccgaaaacgactctttactatcttcgaacgaccccaaaaatcccccgcgacccgcgctactccgaccgcactctacgccttccgcttcgccgacgtacgaaccgccatccgaactacgcgcaccgcctacttcgtaaaaccgacgcccgacgtaataacgatactcga

SEQ ID NO: 84 - DNA, 1085 bp, Artificial Sequence
  converted
tcgaacaccgccaccacgtcgaacgccgattccacgaaacaaacgatacgcgcaactcgaataacgactcgcacgtcgacgaaacgaaaaacgcaaaatacgaccgaaacaacgcgaaccgcgaaaaacccctaaaaccgctcgaaaacaacaaaaaaccgctcccgaactaaaaaaaccgacaccaaacgactataaaaatcgccgcgacccgcgccgcccgcgcactacaaacccaactaaatataaaactcgataaacttcttaacgtcgccgccgacgccgtaaaacaaacaaatacgcgccaacaaactccgcgcctaactctccttatcgcccgcgcgcgcctcgctattaaacgccaaatacgcgtaaaactatacacccgacgacgaatcgaacgaatacaaaaaaacgctcacgaaacccaacttatactactccttaacgccctacttaatcttaaaaatattatcgtcgaaaaaattaaaatcgaaaataaaaaacttaaccaaatcgccgcgcgtatccaaaaaacgaataacgatctcgaacgtattctcgaaacgataatcctccaaactacgattacgcgaaaaaaaaaaactaccgtaaccgatatacgtaacgcccacgaacaaacgactaccccccgcgcccgcgacgaaaaacaaaactaaccccacgataaacgcgttcgaataattaaccgccacgttcaacatactaaaaaacaccgtaacgaactcgacgaacgacgcgacgaacgaaaaacgcacgaccacgaccgaaatattaccccgacgccgcaactaacaaaaaccctaataaataaacccgcacacgactaccaaaccctaaccgaaatccaactacaaaatcttattataattatccgtaaaacgccgcgaatactcgcacgaaacctacgacaactacgaaacgtaacacaacgaactatcgaacaccgaacccacgaccgcctcgacctccaaactcaaattaacgcccgacaactaataaaaacgattaacgaccgccaaatacacgatccccgccgcgccgtccaaaacgaaattattaat

SEQ ID NO: 85 - DNA, 1085 bp, Artificial Sequence
  converted
attaataatttcgttttggacggcgcggcggggatcgtgtatttggcggtcgttaatcgtttttattagttgtcgggcgttaatttgagtttggaggtcgaggcggtcgtgggttcggtgttcgatagttcgttgtgttacgtttcgtagttgtcgtaggtttcgtgcgagtattcgcggcgttttacggataattataataagattttgtagttggatttcggttagggtttggtagtcgtgtgcgggtttatttattagggtttttgttagttgcggcgtcggggtaatatttcggtcgtggtcgtgcgtttttcgttcgtcgcgtcgttcgtcgagttcgttacggtgttttttagtatgttgaacgtggcggttaattattcgaacgcgtttatcgtggggttagttttgtttttcgtcgcgggcgcggggggtagtcgtttgttcgtgggcgttacgtatatcggttacggtagtttttttttttcgcgtaatcgtagtttggaggattatcgtttcgagaatacgttcgagatcgttattcgttttttggatacgcgcggcgatttggttaagttttttattttcgattttaattttttcgacgataatatttttaagattaagtagggcgttaaggagtagtataagttgggtttcgtgagcgtttttttgtattcgttcgattcgtcgtcgggtgtatagttttacgcgtatttggcgtttaatagcgaggcgcgcgcgggcgataaggagagttaggcgcggagtttgttggcgcgtatttgtttgttttacggcgtcggcggcgacgttaagaagtttatcgagttttatatttagttgggtttgtagtgcgcgggcggcgcgggtcgcggcgatttttatagtcgtttggtgtcggtttttttagttcgggagcggttttttgttgttttcgagcggttttaggggtttttcgcggttcgcgttgtttcggtcgtattttgcgtttttcgtttcgtcgacgtgcgagtcgttattcgagttgcgcgtatcgtttgtttcgtggaatcggcgttcgacgtggtggcggtgttcga

SEQ ID NO: 86 - DNA, 85 bp, Homo sapiens
gaagagcttggccaggtcgccgcgcgtgtccagggagcggatggcgatctcgggcgtgttctcgaagcggtggtcctccaggctg

SEQ ID NO: 87 - DNA, 85 bp, Artificial Sequence
  converted
gaagagtttggttaggtcgtcgcgcgtgtttagggagcggatggcgatttcgggcgtgttttcgaagcggtggttttttaggttg

SEQ ID NO: 88 - DNA, 85 bp, Artificial Sequence
  converted
caacctaaaaaaccaccgcttcgaaaacacgcccgaaatcgccatccgctccctaaacacgcgcgacgacctaaccaaactcttc

SEQ ID NO: 89 - DNA, 85 bp, Artificial Sequence
  converted
aaaaaacttaaccaaatcgccgcgcgtatccaaaaaacgaataacgatctcgaacgtattctcgaaacgataatcctccaaacta

SEQ ID NO: 90 - DNA, 85 bp, Artificial Sequence
  converted
tagtttggaggattatcgtttcgagaatacgttcgagatcgttattcgttttttggatacgcgcggcgatttggttaagtttttt

SEQ ID NO: 91 - DNA, 1120 bp, Homo sapiens
ccgaaaaacacgttctaggcgccgggattccagatacctgggaaatagagtgcacgcagctgttgagaggcctcgcgcttggcttctcctatcactgaggcgcagaggtgctgtggacagcccagacccacacggcgcccgaggtgaaacagaaccctcagtctccctatgaggccactggcactctcggctgtccccagagctctccgacttagagctgaatgcaaagtaagcgctcgaaatgcagaagtagccggggccgcccacggcacctgcctcgctcggggcgagagaagacgccaggctgaggtcccagcgacctcaggcaccagctccgaaggagggcggggagaccgcaaaggggaagtgcccggagggccaacggcccccgcgcaccctgcgcccctctgaagcgcgccgcctccccgcgccggggactgggacctgcctctggggaatccgcctagaagacggcggcggactggggtcgggcactctccagggctgtcaggccctccccagccctgcacctgccgcgccgccccacctcgccaggaagtctcagagaccccggggatggggtgggagcgccttcccatcgcgggctcaaaaagaaggaaggacgcccccaggggtcgtagaaggaggactagctccaagccacaactttcttcggacccaaggcaggccggctggggctccgcgcctacacggcccctggcgggggtccgcgcgccccgggagccccgcggctcggggaggaaagaggagacaagagacaggcgaggattacggggctgacccagccggggtagggaccatcgtggaaaaactttggcgaggtggggggacgcggaaagagagcggcccgcgccctgcaccttgcgccgggcatcccgcgccagtgcctcgctcccagtgccccgcgccccgcgccccgcgccttgccttcaccccgggccagctgcatcgcgcccgcgccgcaggaaccgtggagttggaaagtgggggcgccgcggctggggggctgcttcagctgcgcctcggccagcgatcggcgggccgggctcaaatccagccaggctgggcaggcggtggccgcgcgactggggaccgggcg

SEQ ID NO: 92 - DNA, 1120 bp, Artificial Sequence
  converted
tcgaaaaatacgttttaggcgtcgggattttagatatttgggaaatagagtgtacgtagttgttgagaggtttcgcgtttggttttttttattattgaggcgtagaggtgttgtggatagtttagatttatacggcgttcgaggtgaaatagaatttttagtttttttatgaggttattggtattttcggttgtttttagagtttttcgatttagagttgaatgtaaagtaagcgttcgaaatgtagaagtagtcggggtcgtttacggtatttgtttcgttcggggcgagagaagacgttaggttgaggttttagcgattttaggtattagtttcgaaggagggcggggagatcgtaaaggggaagtgttcggagggttaacggttttcgcgtattttgcgtttttttgaagcgcgtcgttttttcgcgtcggggattgggatttgtttttggggaattcgtttagaagacggcggcggattggggtcgggtattttttagggttgttaggttttttttagttttgtatttgtcgcgtcgttttatttcgttaggaagttttagagatttcggggatggggtgggagcgtttttttatcgcgggtttaaaaagaaggaaggacgtttttaggggtcgtagaaggaggattagttttaagttataatttttttcggatttaaggtaggtcggttggggtttcgcgtttatacggtttttggcgggggttcgcgcgtttcgggagtttcgcggttcggggaggaaagaggagataagagataggcgaggattacggggttgatttagtcggggtagggattatcgtggaaaaattttggcgaggtggggggacgcggaaagagagcggttcgcgttttgtattttgcgtcgggtatttcgcgttagtgtttcgtttttagtgtttcgcgtttcgcgtttcgcgttttgtttttatttcgggttagttgtatcgcgttcgcgtcgtaggaatcgtggagttggaaagtgggggcgtcgcggttggggggttgttttagttgcgtttcggttagcgatcggcgggtcgggtttaaatttagttaggttgggtaggcggtggtcgcgcgattggggatcgggcg

SEQ ID NO: 93 - DNA, 1120 bp, Artificial Sequence
  converted
cgcccgatccccaatcgcgcgaccaccgcctacccaacctaactaaatttaaacccgacccgccgatcgctaaccgaaacgcaactaaaacaaccccccaaccgcgacgcccccactttccaactccacgattcctacgacgcgaacgcgatacaactaacccgaaataaaaacaaaacgcgaaacgcgaaacgcgaaacactaaaaacgaaacactaacgcgaaatacccgacgcaaaatacaaaacgcgaaccgctctctttccgcgtccccccacctcgccaaaatttttccacgataatccctaccccgactaaatcaaccccgtaatcctcgcctatctcttatctcctctttcctccccgaaccgcgaaactcccgaaacgcgcgaacccccgccaaaaaccgtataaacgcgaaaccccaaccgacctaccttaaatccgaaaaaaattataacttaaaactaatcctccttctacgacccctaaaaacgtccttccttctttttaaacccgcgataaaaaaacgctcccaccccatccccgaaatctctaaaacttcctaacgaaataaaacgacgcgacaaatacaaaactaaaaaaaacctaacaaccctaaaaaatacccgaccccaatccgccgccgtcttctaaacgaattccccaaaaacaaatcccaatccccgacgcgaaaaaacgacgcgcttcaaaaaaacgcaaaatacgcgaaaaccgttaaccctccgaacacttcccctttacgatctccccgccctccttcgaaactaatacctaaaatcgctaaaacctcaacctaacgtcttctctcgccccgaacgaaacaaataccgtaaacgaccccgactacttctacatttcgaacgcttactttacattcaactctaaatcgaaaaactctaaaaacaaccgaaaataccaataacctcataaaaaaactaaaaattctatttcacctcgaacgccgtataaatctaaactatccacaacacctctacgcctcaataataaaaaaaaccaaacgcgaaacctctcaacaactacgtacactctatttcccaaatatctaaaatcccgacgcctaaaacgtatttttcga

SEQ ID NO: 94 - DNA, 1120 bp, Artificial Sequence
  converted
ccgaaaaacacgttctaaacgccgaaattccaaatacctaaaaaataaaatacacgcaactattaaaaaacctcgcgcttaacttctcctatcactaaaacgcaaaaatactataaacaacccaaacccacacgacgcccgaaataaaacaaaaccctcaatctccctataaaaccactaacactctcgactatccccaaaactctccgacttaaaactaaatacaaaataaacgctcgaaatacaaaaataaccgaaaccgcccacgacacctacctcgctcgaaacgaaaaaaaacgccaaactaaaatcccaacgacctcaaacaccaactccgaaaaaaaacgaaaaaaccgcaaaaaaaaaatacccgaaaaaccaacgacccccgcgcaccctacgcccctctaaaacgcgccgcctccccgcgccgaaaactaaaacctacctctaaaaaatccgcctaaaaaacgacgacgaactaaaatcgaacactctccaaaactatcaaaccctccccaaccctacacctaccgcgccgccccacctcgccaaaaaatctcaaaaaccccgaaaataaaataaaaacgccttcccatcgcgaactcaaaaaaaaaaaaaaacgcccccaaaaatcgtaaaaaaaaaactaactccaaaccacaactttcttcgaacccaaaacaaaccgactaaaactccgcgcctacacgacccctaacgaaaatccgcgcgccccgaaaaccccgcgactcgaaaaaaaaaaaaaaaacaaaaaacaaacgaaaattacgaaactaacccaaccgaaataaaaaccatcgtaaaaaaactttaacgaaataaaaaaacgcgaaaaaaaaacgacccgcgccctacaccttacgccgaacatcccgcgccaatacctcgctcccaataccccgcgccccgcgccccgcgccttaccttcaccccgaaccaactacatcgcgcccgcgccgcaaaaaccgtaaaattaaaaaataaaaacgccgcgactaaaaaactacttcaactacgcctcgaccaacgatcgacgaaccgaactcaaatccaaccaaactaaacaaacgataaccgcgcgactaaaaaccgaacg

SEQ ID NO: 95 - DNA, 1120 bp, Artificial Sequence
  converted
cgttcggtttttagtcgcgcggttatcgtttgtttagtttggttggatttgagttcggttcgtcgatcgttggtcgaggcgtagttgaagtagttttttagtcgcggcgtttttattttttaattttacggtttttgcggcgcgggcgcgatgtagttggttcggggtgaaggtaaggcgcggggcgcggggcgcggggtattgggagcgaggtattggcgcgggatgttcggcgtaaggtgtagggcgcgggtcgtttttttttcgcgtttttttatttcgttaaagtttttttacgatggtttttatttcggttgggttagtttcgtaattttcgtttgttttttgttttttttttttttttcgagtcgcggggttttcggggcgcgcggattttcgttaggggtcgtgtaggcgcggagttttagtcggtttgttttgggttcgaagaaagttgtggtttggagttagttttttttttacgatttttgggggcgtttttttttttttttgagttcgcgatgggaaggcgtttttattttattttcggggtttttgagattttttggcgaggtggggcggcgcggtaggtgtagggttggggagggtttgatagttttggagagtgttcgattttagttcgtcgtcgttttttaggcggattttttagaggtaggttttagttttcggcgcggggaggcggcgcgttttagaggggcgtagggtgcgcgggggtcgttggtttttcgggtattttttttttgcggttttttcgttttttttcggagttggtgtttgaggtcgttgggattttagtttggcgttttttttcgtttcgagcgaggtaggtgtcgtgggcggtttcggttatttttgtatttcgagcgtttattttgtatttagttttaagtcggagagttttggggatagtcgagagtgttagtggttttatagggagattgagggttttgttttatttcgggcgtcgtgtgggtttgggttgtttatagtatttttgcgttttagtgataggagaagttaagcgcgaggttttttaatagttgcgtgtattttattttttaggtatttggaatttcggcgtttagaacgtgtttttcgg

SEQ ID NO: 96 - DNA, 1103 bp, Homo sapiens
aaagtgcctggtaagtaaatgtggatttattagtaaaataagcctagctgcagattcctcatccagatgttcctgaaagaaggcccctggctctgtcttcccggctgtttaaaagaatcattgtttctaattctttcctcccattgtactaagatcaactgtggcaggaactgggtccccctccaggacaaattgttgtttgtcagctgattggttcatacattcattcatcatttacaaggtacttactatatccccaggctgccgtagacacagcctttgctctcccgaaaaacacgttctaggcgccgggattccagatacctgggaaatagagtgcacgcagctgttgagaggcctcgcgcttggcttctcctatcactgaggcgcagaggtgctgtggacagcccagacccacacggcgcccgaggtgaaacagaaccctcagtctccctatgaggccactggcactctcggctgtccccagagctctccgacttagagctgaatgcaaagtaagcgctcgaaatgcagaagtagccggggccgcccacggcacctgcctcgctcggggcgagagaagacgccaggctgaggtcccagcgacctcaggcaccagctccgaaggagggcggggagaccgcaaaggggaagtgcccggagggccaacggcccccgcgcaccctgcgcccctctgaagcgcgccgcctccccgcgccggggactgggacctgcctctggggaatccgcctagaagacggcggcggactggggtcgggcactctccagggctgtcaggccctccccagccctgcacctgccgcgccgccccacctcgccaggaagtctcagagaccccggggatggggtgggagcgccttcccatcgcgggctcaaaaagaaggaaggacgcccccaggggtcgtagaaggaggactagctccaagccacaactttcttcggacccaaggcaggccggctggggctccgcgcctacacggcccctggcgggggtccgcgcgccccgggagccccgcggctcggggaggaaagaggagacaagagacaggcgaggattacggggctgacccagccggggtagg

SEQ ID NO: 97 - DNA, 1103 bp, Artificial Sequence
  converted
aaagtgtttggtaagtaaatgtggatttattagtaaaataagtttagttgtagattttttatttagatgtttttgaaagaaggtttttggttttgttttttcggttgtttaaaagaattattgtttttaattttttttttttattgtattaagattaattgtggtaggaattgggtttttttttaggataaattgttgtttgttagttgattggtttatatatttatttattatttataaggtatttattatatttttaggttgtcgtagatatagtttttgttttttcgaaaaatacgttttaggcgtcgggattttagatatttgggaaatagagtgtacgtagttgttgagaggtttcgcgtttggttttttttattattgaggcgtagaggtgttgtggatagtttagatttatacggcgttcgaggtgaaatagaatttttagtttttttatgaggttattggtattttcggttgtttttagagtttttcgatttagagttgaatgtaaagtaagcgttcgaaatgtagaagtagtcggggtcgtttacggtatttgtttcgttcggggcgagagaagacgttaggttgaggttttagcgattttaggtattagtttcgaaggagggcggggagatcgtaaaggggaagtgttcggagggttaacggttttcgcgtattttgcgtttttttgaagcgcgtcgttttttcgcgtcggggattgggatttgtttttggggaattcgtttagaagacggcggcggattggggtcgggtattttttagggttgttaggttttttttagttttgtatttgtcgcgtcgttttatttcgttaggaagttttagagatttcggggatggggtgggagcgtttttttatcgcgggtttaaaaagaaggaaggacgtttttaggggtcgtagaaggaggattagttttaagttataatttttttcggatttaaggtaggtcggttggggtttcgcgtttatacggtttttggcgggggttcgcgcgtttcgggagtttcgcggttcggggaggaaagaggagataagagataggcgaggattacggggttgatttagtcggggtagg

SEQ ID NO: 98 - DNA, 1103 bp, Artificial Sequence
  converted
cctaccccgactaaatcaaccccgtaatcctcgcctatctcttatctcctctttcctccccgaaccgcgaaactcccgaaacgcgcgaacccccgccaaaaaccgtataaacgcgaaaccccaaccgacctaccttaaatccgaaaaaaattataacttaaaactaatcctccttctacgacccctaaaaacgtccttccttctttttaaacccgcgataaaaaaacgctcccaccccatccccgaaatctctaaaacttcctaacgaaataaaacgacgcgacaaatacaaaactaaaaaaaacctaacaaccctaaaaaatacccgaccccaatccgccgccgtcttctaaacgaattccccaaaaacaaatcccaatccccgacgcgaaaaaacgacgcgcttcaaaaaaacgcaaaatacgcgaaaaccgttaaccctccgaacacttcccctttacgatctccccgccctccttcgaaactaatacctaaaatcgctaaaacctcaacctaacgtcttctctcgccccgaacgaaacaaataccgtaaacgaccccgactacttctacatttcgaacgcttactttacattcaactctaaatcgaaaaactctaaaaacaaccgaaaataccaataacctcataaaaaaactaaaaattctatttcacctcgaacgccgtataaatctaaactatccacaacacctctacgcctcaataataaaaaaaaccaaacgcgaaacctctcaacaactacgtacactctatttcccaaatatctaaaatcccgacgcctaaaacgtatttttcgaaaaaacaaaaactatatctacgacaacctaaaaatataataaataccttataaataataaataaatatataaaccaatcaactaacaaacaacaatttatcctaaaaaaaaacccaattcctaccacaattaatcttaatacaataaaaaaaaaaaattaaaaacaataattcttttaaacaaccgaaaaaacaaaaccaaaaaccttctttcaaaaacatctaaataaaaaatctacaactaaacttattttactaataaatccacatttacttaccaaacacttt

SEQ ID NO: 99 - DNA, 1103 bp, Artificial Sequence
  converted
aaaatacctaataaataaatataaatttattaataaaataaacctaactacaaattcctcatccaaatattcctaaaaaaaaacccctaactctatcttcccgactatttaaaaaaatcattatttctaattctttcctcccattatactaaaatcaactataacaaaaactaaatccccctccaaaacaaattattatttatcaactaattaattcatacattcattcatcatttacaaaatacttactatatccccaaactaccgtaaacacaacctttactctcccgaaaaacacgttctaaacgccgaaattccaaatacctaaaaaataaaatacacgcaactattaaaaaacctcgcgcttaacttctcctatcactaaaacgcaaaaatactataaacaacccaaacccacacgacgcccgaaataaaacaaaaccctcaatctccctataaaaccactaacactctcgactatccccaaaactctccgacttaaaactaaatacaaaataaacgctcgaaatacaaaaataaccgaaaccgcccacgacacctacctcgctcgaaacgaaaaaaaacgccaaactaaaatcccaacgacctcaaacaccaactccgaaaaaaaacgaaaaaaccgcaaaaaaaaaatacccgaaaaaccaacgacccccgcgcaccctacgcccctctaaaacgcgccgcctccccgcgccgaaaactaaaacctacctctaaaaaatccgcctaaaaaacgacgacgaactaaaatcgaacactctccaaaactatcaaaccctccccaaccctacacctaccgcgccgccccacctcgccaaaaaatctcaaaaaccccgaaaataaaataaaaacgccttcccatcgcgaactcaaaaaaaaaaaaaaacgcccccaaaaatcgtaaaaaaaaaactaactccaaaccacaactttcttcgaacccaaaacaaaccgactaaaactccgcgcctacacgacccctaacgaaaatccgcgcgccccgaaaaccccgcgactcgaaaaaaaaaaaaaaaacaaaaaacaaacgaaaattacgaaactaacccaaccgaaataaa

SEQ ID NO: 100 - DNA, 1103 bp, Artificial Sequence
  converted
tttatttcggttgggttagtttcgtaattttcgtttgttttttgttttttttttttttttcgagtcgcggggttttcggggcgcgcggattttcgttaggggtcgtgtaggcgcggagttttagtcggtttgttttgggttcgaagaaagttgtggtttggagttagttttttttttacgatttttgggggcgtttttttttttttttgagttcgcgatgggaaggcgtttttattttattttcggggtttttgagattttttggcgaggtggggcggcgcggtaggtgtagggttggggagggtttgatagttttggagagtgttcgattttagttcgtcgtcgttttttaggcggattttttagaggtaggttttagttttcggcgcggggaggcggcgcgttttagaggggcgtagggtgcgcgggggtcgttggtttttcgggtattttttttttgcggttttttcgttttttttcggagttggtgtttgaggtcgttgggattttagtttggcgttttttttcgtttcgagcgaggtaggtgtcgtgggcggtttcggttatttttgtatttcgagcgtttattttgtatttagttttaagtcggagagttttggggatagtcgagagtgttagtggttttatagggagattgagggttttgttttatttcgggcgtcgtgtgggtttgggttgtttatagtatttttgcgttttagtgataggagaagttaagcgcgaggttttttaatagttgcgtgtattttattttttaggtatttggaatttcggcgtttagaacgtgtttttcgggagagtaaaggttgtgtttacggtagtttggggatatagtaagtattttgtaaatgatgaatgaatgtatgaattaattagttgataaataataatttgttttggagggggatttagtttttgttatagttgattttagtataatgggaggaaagaattagaaataatgatttttttaaatagtcgggaagatagagttaggggtttttttttaggaatatttggatgaggaatttgtagttaggtttattttattaataaatttatatttatttattaggtatttt

SEQ ID NO: 101 - DNA, 103 bp, Homo sapiens
agagctgaatgcaaagtaagcgctcgaaatgcagaagtagccggggccgcccacggcacctgcctcgctcggggcgagagaagacgccaggctgaggtcccag

SEQ ID NO: 102 - DNA, 103 bp, Artificial Sequence
  converted
agagttgaatgtaaagtaagcgttcgaaatgtagaagtagtcggggtcgtttacggtatttgtttcgttcggggcgagagaagacgttaggttgaggttttag

SEQ ID NO: 103 - DNA, 103 bp, Artificial Sequence
  converted
ctaaaacctcaacctaacgtcttctctcgccccgaacgaaacaaataccgtaaacgaccccgactacttctacatttcgaacgcttactttacattcaactct

SEQ ID NO: 104 - DNA, 103 bp, Artificial Sequence
  converted
aaaactaaatacaaaataaacgctcgaaatacaaaaataaccgaaaccgcccacgacacctacctcgctcgaaacgaaaaaaaacgccaaactaaaatcccaa

SEQ ID NO: 105 - DNA, 103 bp, Artificial Sequence
  converted
ttgggattttagtttggcgttttttttcgtttcgagcgaggtaggtgtcgtgggcggtttcggttatttttgtatttcgagcgtttattttgtatttagtttt

SEQ ID NO: 106 - DNA, 19 bp, Artificial Sequence
  primer
tgtttagttttttgttggc

SEQ ID NO: 107 - DNA, 20 bp, Artificial Sequence
  primer
tggagatttttgtaatagtc

SEQ ID NO: 108 - DNA, 19 bp, Artificial Sequence
  primer
tgttttgttttttaggagc

SEQ ID NO: 109 - DNA, 18 bp, Artificial Sequence
  primer
tagggagaggtgggtagc

SEQ ID NO: 110 - DNA, 22 bp, Artificial Sequence
  primer
ttaataaaacaacaattacctc

SEQ ID NO: 111 - DNA, 18 bp, Artificial Sequence
  primer
gaagagtttggttaggtc

SEQ ID NO: 112 - DNA, 19 bp, Artificial Sequence
  primer
ctaaaacctcaacctaacg

SEQ ID NO: 113 - DNA, 20 bp, Artificial Sequence
  primer
taaacttaaacttccaaacg

SEQ ID NO: 114 - DNA, 17 bp, Artificial Sequence
  primer
ccctccttacaaaaccg

SEQ ID NO: 115 - DNA, 17 bp, Artificial Sequence
  primer
actaccaaccttccccg

SEQ ID NO: 116 - DNA, 20 bp, Artificial Sequence
  primer
atatacaaaacaacttcacg

SEQ ID NO: 117 - DNA, 18 bp, Artificial Sequence
  primer
ttttagggaagagaagcg

SEQ ID NO: 118 - DNA, 18 bp, Artificial Sequence
  primer
caacctaaaaaaccaccg

SEQ ID NO: 119 - DNA, 21 bp, Artificial Sequence
  primer
agagttgaatgtaaagtaagc

(19) United States
(12) Patent Application Publication
(10) Pub. No.: US 2023/0287509 A1
(43) Pub. Date: Sep. 14, 2023

(54) METHODS FOR DETECTING LIVER CANCER

(71) Applicant: EPIGENOMICS AG, Berlin (DE)

(72) Inventors: Jörn LEWIN, Berlin (DE); Denise KOTTWITZ, Berlin (DE)

(21) Appl. No.: 18/015,912

(22) PCT Filed: Jul. 21, 2021
(86) PCT No.: PCT/EP2021/070362

§ 371 Date: Jan. 12, 2023

(30) Foreign Application Priority Data
Jul. 21, 2020 (EP) 20187072.2

(51) Int. Cl. C12Q 1/6886

(52) U.S. Cl. CPC C12Q 1/6886; C12Q 2600/154; C12Q 2600/16

(57) ABSTRACT

The present invention relates to the field of pharmacogenomics and in particular to detecting the presence or absence of methylated genomic DNA derived from liver cancer cells in biological samples such as body fluids that contain circulating DNA from the cancer cells. This detection is useful for an early and reliable diagnosis of liver cancer and the invention provides methods and oligonucleotides suitable for this purpose.

### RELATED APPLICATIONS

This application is a 35 U.S.C. § 371 filing of International Patent Application No. PCT/EP2021/070362, filed Jul. 21, 2021, which claims priority to European Patent Application No. 20187072.2, filed Jul. 21, 2020, the entire disclosures of which are hereby incorporated herein by reference.

### SEQUENCE LISTING

The instant application contains a Sequence Listing which has been submitted electronically in ASCII format and is hereby incorporated by reference in its entirety. Said ASCII copy, created on Jan. 9, 2023, is named 738110_EPG9-077_US_ST25.txt and is 144,183 bytes in size.

### FIELD OF THE INVENTION

The present invention relates to the field of pharmacogenomics and in particular to detecting the presence or absence of methylated genomic DNA derived from liver cancer cells in biological samples such as body fluids that contain circulating DNA from the cancer cells. This detection is useful for an early and reliable diagnosis of liver cancer and the invention provides methods and oligonucleotides suitable for this purpose.

### BACKGROUND OF THE INVENTION

Liver cancer (LC) encompasses tumors originating from the liver, and its most common type is hepatocellular carcinoma (HCC), which is the most common cause of death in patients with cirrhosis. It is the sixth most common cancer worldwide, and the usual outcome is poor, because only 10-20% of carcinomas can be removed completely by surgery. Without complete removal of the carcinoma, patients usually die within 3 to 6 months. There is currently no standard or routine test for liver cancer, the most commonly used tests are ultrasound, CT scan and biomarker (alpha-fetoprotein) tests. While tests like ultrasound and CT scan are prone to miss early stages of liver cancer, alpha-fetoprotein is also elevated in cirrhotic liver tissue, making distinguishing between liver cancer and liver cirrhosis difficult.

DNA methylation patterns are largely modified in cancer cells and can therefore be used to distinguish cancer cells from normal tissues. As such, DNA methylation patterns are being used to diagnose all sorts of cancers. One of the challenges is identifying genes or genomic regions that (i) are abnormally methylated in LC and (ii) provide for a diagnostic power that is suitable for detecting LC, i.e. which provide for a sufficient sensitivity and specificity.

It was the goal of the inventors to provide further genes or genomic regions that are abnormally methylated in LC and that also have good and ideally improved sensitivity and/or specificity. It was also the goal of the inventors to provide combinations of such genes or genomic regions that are particularly suitable for detecting LC. Particular emphasis was thereby put on detection using body fluid samples, since their use allows minimally invasive screening of large, e.g. at-risk, populations.

The less advanced LC is, the better the treatment options and the chances of curing the patient are. Thus, it is highly desirable to diagnose it as early and reliably as possible with tests subjects do not hesitate to undergo.

### SUMMARY OF THE INVENTION

In a first aspect, the present invention relates to a method of detecting DNA methylation, comprising the step of detecting DNA methylation within at least one genomic DNA polynucleotide selected from the group consisting of polynucleotides having a sequence comprised in SEQ ID NO: 1 (mASCL2), SEQ ID NO: 21 (mLDHB), SEQ ID NO: 36 (mLGALS3), SEQ ID NO: 46 (mLOXL3), SEQ ID NO: 61 (mOSR1), SEQ ID NO: 76 (mPLXND1), or SEQ ID NO: 91 and/or SEQ ID NO: 96 (mRASSF2) in a subject's biological sample comprising genomic DNA, wherein the genomic DNA may comprise DNA derived from liver cancer (LC) cells.

In a second aspect, the invention relates to a method for detecting the presence or absence of LC in a subject, comprising detecting DNA methylation according to the method of the first aspect, wherein the presence of detected methylated genomic DNA indicates the presence of LC and the absence of detected methylated genomic DNA indicates the absence of LC.

In a third aspect, the present invention relates to an oligonucleotide selected from the group consisting of a primer and a probe, comprising a sequence that is substantially identical to a stretch of contiguous nucleotides of one of SEQ ID NOs 2-5 (mASCL2), 22-25 (mLDHB), 37-40 (mLGALS3), 47-50 (mLOXL3), 62-65 (mOSR1), 77-80 (mPLXND1), or 92-95 and/or 97-100 (mRASSF2).

In a fourth aspect, the present invention relates to a kit comprising at least a first and a second oligonucleotide of the third aspect.

In a fifth aspect, the present invention relates to the use of the method of the first aspect, of the oligonucleotide of the third aspect or of the kit the fourth aspect for the detection of LC or for monitoring a subject having an increased risk of developing LC, suspected of having LC or that has had LC.

In a sixth aspect, the present invention relates to the method of the first or the second aspect, or the use of the fifth aspect, comprising a step of treating LC of a subject for which the DNA methylation is detected in its biological sample.

### LEGENDS TO THE FIGURES

FIG. 1: Map of target regions. See Table 3 for an explanation of the SEQ ID NOs.

FIG. 2: Single marker performance and methylation differences. Grey squares show comethylation for marker A-G (CoM number of completely methylated fragments in relation to all amplified DNA in an assay as detected by reads matching an assay) normalized to a range of 0 to 1 in a linear scale by greyscale color or in a logarithmic scale by size as laid out in the legend at the bottom. Plasma samples for 41 hepatocellular carcinoma (HCC) patients and 46 individuals with no evidence of HCC but with liver cirrhosis (LCi) are vertically grouped into their two diagnostic groups. Numbers at the bottom are area under the curves from receiver operating characteristic curves. Grey bars and numbers on the right are the sum of all fully methylated molecules (rounded to 1000) as amplified in the PCR and normalized by total amount of amplified DNA measured for a sample. Markers are A: mASCL2, B: mLDHB, C: mLGALS3, D: mLOXL3, E: mOSR1, F: mPLXND1, G: mRASSF2.

FIG. 3: Receiver operating characteristic curves (ROCs) for seven markers and two exemplary marker combinations by logistic regression analysis. The curves show the relation of the sensitivity (y-axis) to the specificity (x-axis). Areas under the curve (AUC) are written at the bottom right of the plotting area.

FIG. 4: ROC curves for seven markers combination as measured in an NGS panel (straight line), AFP (dashed line) and combination of 7 marker NGS panel with AFP (dotted line) by logistic regression for 60 HCC vs. 102 controls in a testing setting (Example 2). The curves show the relation of the true positive rate (TPR) on the y-axis to the false positive rate (FPR) on the x-axis. Areas under the curve (AUC) are written at the bottom right of the plotting area, selected sensitivity/specificity pairs on the curves are reported for 3+/7 markers positive, AFP>=20 ng/mL and some corresponding points at FPR of 3% (Specificity of 97%). The grey point describes the sensitivity/specificity for the combination of 3+/7 positive NGS markers OR AFP>=20 ng/mL.

FIG. 5: ROC curves for four markers combination as measured by Real-time PCR (straight line), AFP (dashed line) and combination of 4 marker Real-time PCR with AFP by logistic regression (dotted line) for 60 HCC vs. 102 controls in a testing setting (Example 3). The curves show the relation of the true positive rate (TPR) on the y-axis to the false positive rate (FPR) on the x-axis. Areas under the curve (AUC) are written at the bottom right of the plotting area. The grey point describes the sensitivity of 75% at specificity of 86% for the combination of 2+/4 called Real-time PCR markers OR AFP>=20 ng/mL.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations) “, Leuenberger, H. G. W, Nagel, B. and Kolb′, H. eds. (1995), Helvetica Chimica Acta, CH-4010 Basel, Switzerland).

Several documents are cited throughout the text of this specification. Each of the documents cited herein (including all patents, patent applications, scientific publications, manufacturers' specifications, instructions etc.), whether supra or infra, is hereby incorporated by reference in its entirety.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step. In preferred embodiments, “comprise” can mean “consist of′. As used in this specification and the appended claims, the singular forms “a”, “an”, and “the” include plural referents, unless the content clearly dictates otherwise.

### Aspects of the Invention and Particular Embodiments Thereof

In a first aspect, the present invention relates to a method of detecting DNA methylation, comprising the step of detecting DNA methylation within at least one genomic DNA polynucleotide selected from the group consisting of polynucleotides having a sequence comprised in SEQ ID NO: 1 (mASCL2), SEQ ID NO: 21 (mLDHB), SEQ ID NO: 36 (mLGALS3), SEQ ID NO: 46 (mLOXL3), SEQ ID NO: 61 (mOSR1), SEQ ID NO: 76 (mPLXND1), or SEQ ID NO: 91 and/or SEQ ID NO: 96 (mRASSF2) in a subject's biological sample comprising genomic DNA. Specifically, the genomic DNA may comprise DNA derived from liver cancer (LC) cells. Preferably, the genomic DNA, in particular the genomic DNA derived from LC cells, is cell-free DNA. The phrase “the genomic DNA may comprise DNA derived from liver cancer (LC) cells” does, in a preferred embodiment, mean that the subject has an increased risk of LC, is suspected of having LC or has had LC (i.e. has been treated to remove any detectable sign of LC, but is suspected to relapse). In a preferred embodiment, an increased risk of LC (i.e. increased risk of developing LC) means that the subject has one or more LC risk factors. Preferably, LC risk factors are selected from the group consisting of chronic infection with HBV and/or HCV, cirrhosis, liver disease (preferably an inheritable liver disease, e.g. hemochromatosis, Wilson's disease tyrosinemia, alpha1-antitrypsin deficiency, porphyria cutanea tarda, or glycogen storage disease), diabetes (preferably type 2), nonalcoholic fatty liver disease, exposure to aflatoxins, and heavy alcohol consumption (i.e. bringing the blood alcohol concentration level to at least 0.08 g/dL at least 5 times per month). A subject at increased risk herein is most preferably a subject having cirrhosis.

Preferably, the method is an in vitro method.

In a preferred embodiment,
the polynucleotide having a sequence comprised in SEQ ID NO: 1 has a sequence comprised in SEQ ID NO: 6, preferably in SEQ ID NO: 11,
the polynucleotide having a sequence comprised in SEQ ID NO: 21 has a sequence comprised in SEQ ID NO: 16, preferably in SEQ ID NO: 26,
the polynucleotide having a sequence comprised in SEQ ID NO: 36 has a sequence comprised in SEQ ID NO: 31, preferably in SEQ ID NO: 41,
the polynucleotide having a sequence comprised in SEQ ID NO: 46 has a sequence comprised in SEQ ID NO: 51, preferably in SEQ ID NO: 56,
the polynucleotide having a sequence comprised in SEQ ID NO: 61 has a sequence comprised in SEQ ID NO: 66, preferably in SEQ ID NO: 71,
the polynucleotide having a sequence comprised in SEQ ID NO: 76 has a sequence comprised in SEQ ID NO: 81, preferably in SEQ ID NO: 86, and/or
the polynucleotide having a sequence comprised in SEQ ID NO: 91 and/or SEQ ID NO: 96 has a sequence comprised in SEQ ID NO: 101.

Preferably, DNA methylation is detected within at least two, more preferably at least three (or at least 4, 5, 6 or in all, wherein larger numbers are preferred to smaller numbers) genomic DNA polynucleotides selected from said group (each polynucleotide corresponding to a different methylation marker). In specific preferred embodiments, methylation is detected for a combination of two markers according to Table 1 or three markers according to Table 2 (the tables showing advantageous AUC values), and optionally one or more further markers of the group consisting of mASCL2, mLDHB, mLGALS3, mLOXL3, mOSR1, mPLXND1 and mRASSF2 (sequences recited as above, including preferred ones). Of the combinations recited in Table 1, those are particularly preferred for which an AUC of at least 0.75, preferably at least 0.80, 0.81, 0.82, 0.83, or 0.84 (higher AUCs preferred to lower ones) is shown in Table 1. Of the combinations recited in Table 2, those are particularly preferred for which an AUC of at least 0.75, preferably at least 0.80, 0.81, 0.82, 0.83, 0.84, 0.85 or 0.86 (higher AUCs preferred to lower ones) is shown in Table 2.

The sequence the polynucleotide has is also referred to herein as the target region or target DNA and may be the sequence of the entire SEQ ID NO, or may be a sequence with a length as specified below in the section “Definitions and further embodiments of the invention”.

In a preferred embodiment, the genomic target DNA (the DNA region within which methylation is detected) comprises at least one CpG dinucleotide, preferably at least 2, 3, 4, or 5, most preferably at least 6 (e.g. at least 10, 15 or 30) CpG dinucleotides. Generally, the methylation of at least one CpG dinucleotide comprised in the genomic DNA is detected, preferably of at least 2, 3, 4, or 5, most preferably at least 6 (e.g. at least 10, 15 or 30) CpG dinucleotides. Furthermore, the methylation of usually all CpG dinucleotides comprised in the genomic target DNA is detected. Nevertheless, it is possible that the methylation detection of a part of the CpG dinucleotides is omitted (a part meaning up to 3, 2 or preferably 1, but never all), for example if the species the subject belongs to (preferably human) has a single polynucleotide polymorphism (SNP) at one or both positions of the CpG dinucleotide.

In one embodiment, the method of the first aspect comprises the steps of
(a) converting cytosine unmethylated in the 5-position to uracil or another base that does not hybridize to guanine in the genomic DNA of the biological sample; and
(b) detecting DNA methylation within the genomic DNA by detecting unconverted cytosine in the converted DNA of step (a).

A preferred way of carrying out the method comprises the steps of
(a) converting cytosine unmethylated in the 5-position to uracil or another base that does not hybridize to guanine in the genomic DNA;
(b) amplifying methylation-specifically a region of the converted DNA;
(c) detecting the presence or absence of DNA amplified in step (b);
wherein the presence or absence of amplified DNA indicates the presence or absence, respectively, of methylated genomic DNA.

In a preferred embodiment, step b) of amplifying comprises the use of at least one oligonucleotide according to the fourth aspect, preferably as a primer. More preferably, it comprises the use of oligonucleotides as comprised in the kit of the fifth aspect.

In a preferred embodiment of the method of the first aspect, the detecting of the DNA methylation comprises determining the amount of methylated genomic DNA. Any means known in the art can be used to detect DNA methylation or determine its amount (see also below for art-known and preferred means). It is preferred that methylation is detected or the amount of methylated genomic DNA is determined by sequencing, in particular next-generation-sequencing (NGS), by real-time PCR or by digital PCR.

Markers mASCL2, mLDHB, mLGALS3, mLOXL3, mOSR1, mPLXND1 and mRASSF2 show consistent comethylation and, thus, the amount of methylation can be determined simply by counting the number of methylated sequences (reads) when determining the amount of methylation by sequencing.

In a preferred embodiment, the biological sample is a liver tissue sample or a liquid biopsy, preferably a blood sample, a sample comprising cell-free DNA from blood (e.g. a urine sample), a blood-derived sample or a saliva sample.

In another preferred embodiment, the subject has an increased risk of developing LC, is suspected of having LC, has had LC or has LC.

In a preferred embodiment, the method further comprises obtaining the alpha-fetoprotein (AFP) blood (preferably plasma) level of the subject. AFP is a major plasma protein that is used as a biomarker for Down syndrome, neural tube defects and other chromosomal abnormalities (all in maternal blood) and for other conditions such as LC including hepatocellular carcinoma, germ cell tumors, yolk sac tumor and ataxia telangiectasia. Used by itself, it lacks the discriminatory power for a reliable diagnosis in particular of LC. The term “obtaining” in the context of the AFP level comprises obtaining pre-existing AFP test results of the subject, and alternatively in vitro determining the AFP level in blood (preferably the biological sample of the subject if it is blood or a blood-derived sample). AFP levels are routinely determined clinically, and the way of determining is not particularly limited, an example is by using an AFP antibody, e.g. in an AFP ELISA. See for instance Shahangian et al., Clin Chem. 1987 April; 33(4):583-6).

Definitions and embodiments described below, in particular under the header ‘Definitions and further embodiments of the invention’ apply to the method of the first aspect.

In a second aspect, the invention relates to a method for detecting the presence or absence of LC in a subject, comprising detecting DNA methylation according to the method of the first aspect, wherein the presence of detected methylated genomic DNA indicates the presence of LC and the absence of detected methylated genomic DNA indicates the absence of LC. Thus, the method of the second aspect useful as a method for diagnosis of LC. The method is also useful as a method for screening a population of subjects for LC.

Preferably, the method is an in vitro method.

The cancer may be of any subtype and stage as defined below, i.e. the presence or absence of any subtype and/or stage can be detected. In a preferred embodiment, the liver cancer (LC) is hepatocellular carcinoma (HCC), i.e. all references herein to liver cancer (LC) are preferably understood as references to hepatocellular carcinoma (HCC).

In a preferred embodiment, the presence of a significant amount of methylated genomic DNA, or of an amount larger than in a control, indicates the presence of LC, and the absence of a significant amount of methylated genomic DNA, or of an amount equal to or smaller than in a control, indicates the absence of LC.

In a particular embodiment, the method of the second aspect further comprises confirming the detection of LC by using one or more further means for detecting LC. The further means may be a cancer marker (or “biomarker”) or a conventional (non-marker) detection means. The cancer marker can for example be a DNA methylation marker, a mutation marker (e.g. SNP), an antigen marker, a protein marker, a miRNA marker, a cancer specific metabolite, or an expression marker (e.g. RNA or protein expression). The conventional means can for example be a biopsy (e.g. visual biopsy examination with or without staining methods for example for protein or expression markers), an imaging technique (e.g. X-ray imaging, CT scan, nuclear imaging such as PET and SPECT, ultrasound, magnetic resonance imaging (MRI), thermography, or endoscopy) or a physical, e.g. tactile examination. It is preferred that it is a biopsy or other means that removes and examines a solid tissue sample of the subject from the tissue for which LC is indicated (i.e. no liquid tissue such as blood).

In a preferred embodiment, the method of the second aspect is for monitoring a subject having an increased risk of developing LC, suspected of having or developing LC or that has had LC, comprising detecting DNA methylation repeatedly, wherein the presence of detected methylated genomic DNA indicates the presence of LC and the absence of detected methylated genomic DNA indicates the absence of LC. Preferably, the detecting of the DNA methylation comprises determining the amount of methylated genomic DNA, wherein an increased amount of methylated genomic DNA in one or more repeated detections of DNA methylation indicates the presence of LC and a constant or decreased amount in repeated detections of DNA methylation indicates the absence of LC.

In a preferred embodiment, the method of the second aspect comprises assessing the AFP blood level of the subject, wherein the presence of detected methylated genomic DNA in combination with an increased AFP blood level indicates the presence of LC and the absence of detected methylated genomic DNA in combination with a normal AFP blood level indicates the absence of LC. Assessing the AFP blood level of a subject is routine in the art, including for the detection and monitoring of LC, see e.g. Chan et al. (Clin Chem., 1986 Jul;32(7):1318-22). Generally, an “increased” level means increased above normal, i.e. “abnormally increased”. Normal therein refers to the AFP level of a person (or average or median of a plurality of persons) not having LC (but optionally having liver cirrhosis), and preferably (i) not having another disorder associated with an increased AFP level (specifically those listed with regard to the first aspect) and/or (ii) not being pregnant. If the subject is pregnant, normal refers to the AFP level of a pregnant person (or average or median of a plurality of pregnant persons). An exemplary value for an increased AFP blood level is >10 μg/L (non-pregnant subject) and, if the subject is pregnant, >420 μg/L. The corresponding exemplary value for a normal AFP blood level is <10 μg/L (non-pregnant subject) and, if the subject is pregnant, ≤420 μg/L. For increasing the diagnostic efficiency, a higher value for an increased AFP blood level can be used, in particular to discriminate LC from liver cirrhosis, e.g. ≥20, ≥50, ≥100, ≥200 or preferably >400 μg/L (non-pregnant subject, corresponding normal levels≤20, ≤50, ≤100, ≤200 or preferably <400 μg/L). Of these, a commonly used and herein preferred value is >20 μg/L (increased)/<20 μg/L (normal). As indicated above, specificity and sensitivity of using AFP levels for detecting LC are not satisfactory, in fact using an AFP test is only optional in the American Association for the Study of Liver Diseases (AASLD) guidelines and not recommended by the European Association for the Study of the Liver (EASL) guidelines due to suboptimal performance (Foerster and Galle, THEP Reports, Vol. 1, Issue 2, 2019, p. 114-119). The inventors found that the combination of the methylation markers of the invention with the AFP blood level increases unexpectedly both specificity and sensitivity, even in a synergistic manner to a surprising extent.

Definitions given and embodiments described with respect to the first aspect apply also to the second aspect, in as far as they are applicable. Also, definitions and embodiments described below, in particular under the header ‘Definitions and further embodiments of the invention’ apply to the method of the second aspect.

In a third aspect, the present invention relates to an oligonucleotide selected from the group consisting of a primer and a probe, comprising a sequence that is substantially identical to a stretch of contiguous nucleotides of one of SEQ ID NOs 2-5 (mASCL2), one of 22-25 (mLDHB), one of 37-40 (mLGALS3), one of 47-50 (mLOXL3), one of 62-65 (mOSR1), one of 77-80 (mPLXND1), or one of 92-95 and/or one of 97-100 (mRASSF2).

In a preferred embodiment,
the sequence that is substantially identical to a stretch of contiguous nucleotides of one of SEQ ID NOs 2-5 is substantially identical to a stretch of contiguous nucleotides of one of SEQ ID NOs 7-10, preferably one of SEQ ID NOs 12-15,
the sequence that is substantially identical to a stretch of contiguous nucleotides of one of SEQ ID NOs 22-25 is substantially identical to a stretch of contiguous nucleotides of one of SEQ ID NOs 17-20, preferably one of SEQ ID NOs 27-30,
the sequence that is substantially identical to a stretch of contiguous nucleotides of one of SEQ ID NOs 37-40 is substantially identical to a stretch of contiguous nucleotides of one of SEQ ID NOs 32-35, preferably one of SEQ ID NOs 42-45,
the sequence that is substantially identical to a stretch of contiguous nucleotides of one of SEQ ID NOs 47-50 is substantially identical to a stretch of contiguous nucleotides of one of SEQ ID NOs 52-55, preferably one of SEQ ID NOs 57-60,
the sequence that is substantially identical to a stretch of contiguous nucleotides of one of SEQ ID NOs 62-65 is substantially identical to a stretch of contiguous nucleotides of one of SEQ ID NOs 67-70, preferably one of SEQ ID NOs 72-75,
the sequence that is substantially identical to a stretch of contiguous nucleotides of one of SEQ ID NOs 77-80 is substantially identical to a stretch of contiguous nucleotides of one of SEQ ID NOs 82-85, preferably one of SEQ ID NOs 87-90, and/or
the sequence that is substantially identical to a stretch of contiguous nucleotides of one of SEQ ID NOs 92-95 and/or one of SEQ ID NOs 97-100 is substantially identical to a stretch of contiguous nucleotides of one of SEQ ID NOs 102-105.

Herein, a sequence that is substantially identical to a stretch of contiguous nucleotides of two (or more) SEQ ID NOs, e.g. of one of SEQ ID NOs 92-95 and one of SEQ ID NOs 97-100, is identical to two (or more) corresponding SEQ ID NOs. “Corresponding” means of the same type of the same methylation marker (e.g. mASCL2) according to Table 3 (the types are genomic reference, C to T (bis 1), rc C to T (bis 1), G to A (bis2 rc) and G to A (bis2 rc) rc).

Generally, the oligonucleotide is bisulfite-specific. Preferably, the oligonucleotide is methylation-specific, more preferably positive methylation-specific.

The oligonucleotide may be a primer or a probe oligonucleotide, preferably it is a primer oligonucleotide. A probe preferably has one or more modifications selected from the group consisting of a detectable label and a quencher, and/or a length of 5-40 nucleotides. A primer preferably has a priming region with a length of 10-40 nucleotides.

Definitions given and embodiments described with respect to the first and second aspect apply also to the third aspect, in as far as they are applicable. Also, definitions and embodiments described below, in particular under the header ‘Definitions and further embodiments of the invention’ apply to the oligonucleotide of the third aspect.

In a fourth aspect, the present invention relates to a kit comprising at least a first and a second oligonucleotide of the third aspect.

In a preferred embodiment, the first and second oligonucleotides are primers forming a primer pair suitable for amplification of DNA having a sequence comprised in one of SEQ ID NOs 2-5 (mASCL2), one of SEQ ID NOs 22-25 (mLDHB), one of SEQ ID NOs 37-40 (mLGALS3), one of SEQ ID NOs 47-50 (mLOXL3), one of SEQ ID NOs 62-65 (mOSR1), one of SEQ ID NOs 77-80 (mPLXND1), or one of SEQ ID NOs 92-95 or one of SEQ ID NOs 97-100 (mRASSF2).

Preferably,
the sequence comprised in one of SEQ ID NOs 2-5 is comprised in one of SEQ ID NOs 7-10, preferably one of SEQ ID NOs 12-15,
the sequence comprised in one of SEQ ID NOs 22-25 is comprised in one of SEQ ID NOs 17-20, preferably one of SEQ ID NOs 27-30,
the sequence comprised in one of SEQ ID NOs 37-40 is comprised in one of SEQ ID NOs 32-35, preferably one of SEQ ID NOs 42-45,
the sequence comprised in one of SEQ ID NOs 47-50 is comprised in one of SEQ ID NOs 52-55, preferably one of SEQ ID NOs 57-60,
the sequence comprised in one of SEQ ID NOs 62-65 is comprised in one of SEQ ID NOs 67-70, preferably one of SEQ ID NOs 72-75,
the sequence comprised in one of SEQ ID NOs 77-80 is comprised in one of SEQ ID NOs 82-85, preferably one of SEQ ID NOs 87-90, and/or
the sequence comprised in one of SEQ ID NOs 92-95 and/or one of SEQ ID NOs 97-100 is comprised in one of SEQ ID NOs 102-105.

Herein, a sequence that is comprised in two (or more) SEQ ID NOs, e.g. of one of SEQ ID NOs 92-95 and/or one of SEQ ID NOs 97-100, is comprised to two (or more) corresponding SEQ ID NOs. “Corresponding” means of the same type of the same methylation marker according to Table 3.

In another preferred embodiment, the kit comprises polynucleotides forming at least two, preferably at least three (or at least 4, 5, 6 or in all, wherein larger numbers are preferred to smaller numbers) such primer pairs, wherein each primer pair is suitable for amplification of DNA having a sequence of a different marker selected from the group consisting of mASCL2, mLDHB, mLGALS3, mLOXL3, mOSR1, mPLXND1 and mRASSF2.

In specific preferred embodiments, the kit comprises polynucleotides forming primer pairs for markers of a combination of two markers according to Table 1 or three markers according to Table 2 (for which advantageous AUC values are shown), and optionally one or more further marker of the group consisting of mASCL2, mLDHB, mLGALS3, mLOXL3, mOSR1, mPLXND1 and mRASSF2.

Of the combinations recited in Table 1, those are particularly preferred for which an AUC of at least 0.75, preferably at least 0.80, 0.81, 0.82, 0.83, or 0.84 (higher AUCs preferred to lower ones) is shown in Table 1. Of the combinations recited in Table 2, those are particularly preferred for which an AUC of at least 0.75, preferably at least 0.80, 0.81, 0.82, 0.83, 0.84, 0.85 or 0.86 (higher AUCs preferred to lower ones) is shown in Table 2.

In a preferred embodiment, the kit also comprises a compound suitable for detecting the AFP level in a sample, e.g. an AFP antibody.

Definitions given and embodiments described with respect to the first, second and third aspect apply also to the fourth aspect, in as far as they are applicable. Also, definitions and embodiments described below, in particular under the header ‘Definitions and further embodiments of the invention’ apply to the kit of the fourth aspect.

In a fifth aspect, the present invention relates to the use of the method of the first aspect, of the oligonucleotide of the third aspect or of the kit the fourth aspect for the detection of LC or for monitoring a subject having an increased risk of developing LC, suspected of having or developing LC or who has had LC. Preferably, the use is an in vitro use.

Definitions given and embodiments described with respect to the first, second, third and fourth aspect apply also to the fifth aspect, in as far as they are applicable. Also, definitions and embodiments described below, in particular under the header ‘Definitions and further embodiments of the invention’ apply to the use of the fifth aspect.

In a sixth aspect, the present invention relates to the method of the first or the second aspect, or the use of the fifth aspect, comprising a step of treating LC of a subject for which the DNA methylation is detected in its biological sample. In other words, the method of the sixth aspect can be described as a method of treatment, comprising the method of the first or the second aspect, or the use of the fifth aspect and a step of treating LC of a subject for which the DNA methylation is detected in its biological sample. It can also be described as a method of treatment, comprising treating LC in a subject for which DNA methylation has been detected according to the method of the first or the second aspect, or the use of the fifth aspect.

Definitions given and embodiments described with respect to the first, second, third, fourth and fifth aspect apply also to the sixth aspect, in as far as they are applicable. Also, definitions and embodiments described below, in particular under the header ‘Definitions and further embodiments of the invention apply to the method of the sixth aspect.

TABLE 1
Combinations of at least two markers comprising markers 1 and 2

| Marker 1 | Marker 2 | AUC |
|---|---|---|
| mASCL2 | mLDHB | 0.743 |
| mASCL2 | mLGALS3 | 0.741 |
| mASCL2 | mLOXL3 | 0.840 |
| mASCL2 | mOSR1 | 0.797 |
| mASCL2 | mPLXND1 | 0.763 |
| mASCL2 | mRASSF2 | 0.784 |
| mLDHB | mLGALS3 | 0.732 |
| mLDHB | mLOXL3 | 0.800 |
| mLDHB | mOSR1 | 0.747 |
| mLDHB | mPLXND1 | 0.729 |
| mLDHB | mRASSF2 | 0.745 |
| mLGALS3 | mLOXL3 | 0.822 |
| mLGALS3 | mOSR1 | 0.758 |
| mLGALS3 | mPLXND1 | 0.740 |
| mLGALS3 | mRASSF2 | 0.785 |
| mLOXL3 | mOSR1 | 0.809 |
| mLOXL3 | mPLXND1 | 0.818 |
| mLOXL3 | mRASSF2 | 0.831 |
| mOSR1 | mPLXND1 | 0.776 |
| mOSR1 | mRASSF2 | 0.754 |
| mPLXND1 | mRASSF2 | 0.771 |

TABLE 2
Combinations of at least three markers
comprising markers 1, 2 and 3

| Marker 1 | Marker 2 | Marker 3 | AUC |
|---|---|---|---|
| mASCL2 | mLDHB | mLGALS3 | 0.745 |
| mASCL2 | mLDHB | mLOXL3 | 0.846 |
| mASCL2 | mLDHB | mOSR1 | 0.800 |
| mASCL2 | mLDHB | mPLXND1 | 0.766 |
| mASCL2 | mLDHB | mRASSF2 | 0.799 |
| mASCL2 | mLGALS3 | mLOXL3 | 0.841 |
| mASCL2 | mLGALS3 | mOSR1 | 0.802 |
| mASCL2 | mLGALS3 | mPLXND1 | 0.767 |
| mASCL2 | mLGALS3 | mRASSF2 | 0.789 |
| mASCL2 | mLOXL3 | mOSR1 | 0.847 |
| mASCL2 | mLOXL3 | mPLXND1 | 0.844 |
| mASCL2 | mLOXL3 | mRASSF2 | 0.862 |
| mASCL2 | mOSR1 | mPLXND1 | 0.814 |
| mASCL2 | mOSR1 | mRASSF2 | 0.812 |
| mASCL2 | mPLXND1 | mRASSF2 | 0.790 |
| mLDHB | mLGALS3 | mLOXL3 | 0.820 |
| mLDHB | mLGALS3 | mOSR1 | 0.770 |
| mLDHB | mLGALS3 | mPLXND1 | 0.752 |
| mLDHB | mLGALS3 | mRASSF2 | 0.777 |
| mLDHB | mLOXL3 | mOSR1 | 0.808 |
| mLDHB | mLOXL3 | mPLXND1 | 0.819 |
| mLDHB | mLOXL3 | mRASSF2 | 0.825 |
| mLDHB | mOSR1 | mPLXND1 | 0.776 |
| mLDHB | mOSR1 | mRASSF2 | 0.766 |
| mLDHB | mPLXND1 | mRASSF2 | 0.765 |
| mLGALS3 | mLOXL3 | mOSR1 | 0.821 |
| mLGALS3 | mLOXL3 | mPLXND1 | 0.834 |
| mLGALS3 | mLOXL3 | mRASSF2 | 0.840 |
| mLGALS3 | mOSR1 | mPLXND1 | 0.786 |
| mLGALS3 | mOSR1 | mRASSF2 | 0.781 |
| mLGALS3 | mPLXND1 | mRASSF2 | 0.777 |
| mLOXL3 | mOSR1 | mPLXND1 | 0.817 |
| mLOXL3 | mOSR1 | mRASSF2 | 0.820 |
| mLOXL3 | mPLXND1 | mRASSF2 | 0.829 |
| mOSR1 | mPLXND1 | mRASSF2 | 0.788 |

### Definitions and Further Embodiments of the Invention

The specification uses a variety of terms and phrases, which have certain meanings as defined below. Preferred meanings are to be construed as preferred embodiments of the aspects of the invention described herein. As such, they and also further embodiments described in the following can be combined with any embodiment of the aspects of the invention and in particular any preferred embodiment of the aspects of the invention described above.

The term “methylated” as used herein refers to a biochemical process involving the addition of a methyl group to cytosine DNA nucleotides. DNA methylation at the 5 position of cytosine, especially in promoter regions, can have the effect of reducing gene expression and has been found in every vertebrate examined. In adult non-gamete cells, DNA methylation typically occurs in a CpG site. The term “CpG site” or “CpG dinucleotide”, as used herein, refers to regions of DNA where a cytosine nucleotide occurs next to a guanine nucleotide in the linear sequence of bases along its length. “CpG” is shorthand for “C-phosphate-G”, that is cytosine and guanine separated by only one phosphate; phosphate links any two nucleosides together in DNA. The “CpG” notation is used to distinguish this linear sequence from the CG base-pairing of cytosine and guanine. Cytosines in CpG dinucleotides can be methylated to form 5-methylcytosine. The term “CpG site” or “CpG site of genomic DNA” is also used with respect to the site of a former (unmethylated) CpG site in DNA in which the unmethylated C of the CpG site was converted to another as described herein (e.g. by bisulfite to uracil). The application provides the genomic sequence of each relevant DNA region as well as the bisulfite converted sequences of each converted strand. CpG sites referred to are always the positions of the CpG sites of the genomic sequence, even if the converted sequence does no longer contain these CpG sites due to the conversion. Specifically, methylation in the context of the present invention means hypermethylation. The term “hypermethylation” refers to an aberrant methylation pattern or status (i.e. the presence or absence of methylation of one or more nucleotides), wherein one or more nucleotides, preferably C(s) of a CpG site(s), are methylated compared to the same genomic DNA of a control, i.e. from a non-cancer cell of the subject or a subject not suffering or having suffered from the cancer the subject is treated for, preferably any cancer (healthy control). The term “control” can also refer to the methylation status, pattern or amount which is the average or median known of or determined from a group of at least 5, preferably at least 10 subjects. In particular, it refers to an increased presence of 5-mCyt at one or a plurality of CpG dinucleotides within a DNA sequence of a test DNA sample, relative to the amount of 5-mCyt found at corresponding CpG dinucleotides within a (healthy) control DNA sample, both samples preferably being of the same type, e.g. both blood plasma, both blood serum, both saliva, or both urine. Hypermethylation as a methylation status/pattern can be determined at one or more CpG site(s). If more than one CpG site is used, hypermethylation can be determined at each site separately or as an average of the CpG sites taken together. Alternatively, all assessed CpG sites must be methylated (comethylation) such that the requirement hypermethylation is fulfilled.

The term “detecting DNA methylation” as used herein refers to at least qualitatively analysing for the presence or absence of methylated target DNA. “Target DNA” refers to a sequence within the genomic DNA polynucleotide (region) that is generally limited in length, but is preferably a length suitable for PCR amplification, e.g. at least 30 to 1000, more preferably 50 to 300 and even more preferably 75 to 200 or 75 to 150 nucleotides long. This includes primer binding sites if the target region is amplified using primers. Methylation is preferably determined at 1 or more, 2 or more, 3 or more, 4 or more, or 5 or more, most preferably 6 or more (e.g. 10 or more, 15 or more, or 30 or more) CpG sites of the target DNA. Usually, the CpG sites analysed are comethylated in cancer, such that also CpG sites of neighbouring DNA are methylated and can be analysed in addition or instead. “At least qualitatively” means that also a quantitative determination of methylated target DNA, if present, can be performed. In fact, it is preferred that detecting of the DNA methylation comprises determining the amount of methylated genomic DNA.

DNA methylation can be detected or its amount can be determined by various means known in the art, e.g. autoradiography, silver staining or ethidium bromide staining, methylation sensitive single nucleotide extension (MS-SNUPE), methyl-binding proteins, antibodies for methylated DNA, methylation-sensitive restriction enzymes etc., preferably by sequencing, e.g. next-generation-sequencing (NGS), or by real-time PCR, e.g. multiplex real-time PCR, or by digital PCR (dPCR). In particular if 3 or more (e.g. 4 or more or 5 or more) different target DNAs (i.e. markers) are examined in parallel, it is preferred that the presence or absence of methylated DNA is detected by sequencing, preferably by NGS.

In a real-time PCR, this is done by detecting a methylation-specific oligonucleotide probe during amplifying the converted (e.g. bisulfite converted) target DNA methylation-specifically using methylation-specific primers or a methylation-specific blocker with methylation-specific primers or preferably methylation-unspecific primers.

Digital PCR (dPCR) is a quantitative PCR in which a PCR reaction mixture is partitioned into individual compartments (e.g. wells or water-in-oil emulsion droplets) resulting in either 1 or 0 targets being present in each compartment. Following PCR amplification, the number of positive vs negative reactions is determined and the quantification is by derived from this result statistically, preferably using Poisson statistics. A preferred dPCR is BEAMing (Beads, Emulsion, Amplification, Magnetics), in which DNA templates (which may be pre-amplified) are amplified using primers bound to magnetic beads present compartmentalized in water-in-oil emulsion droplets. Amplification results in the beads being covered with amplified DNA. The beads are then pooled and amplification is analysed, e.g. using methylation-specific fluorescent probes which can be analyzed by flow cytometry. See for instance Yokoi et al. (Int J Sci. 2017 April; 18(4):735). Applied to methylation analysis, the method is also known as Methyl BEAMing.

A detection by sequencing is preferably a detection by NGS. Therein, the converted methylated target DNA is amplified, preferably methylation-specifically (the target DNA is amplified if it is methylated, in other words if cytosines of the CpG sites are not converted). This can be achieved by bisulfite-specific primers which are methylation-specific. Then, the amplified sequences are sequenced and subsequently counted. The ratio of sequences derived from converted methylated DNA (identified in the sequences by CpG sites) and sequences derived from converted unmethylated DNA is calculated, resulting in a (relative) amount of methylated target DNA.

The term “next-generation-sequencing” (NGS, also known as 2^{nd} or 3^{rd} generation sequencing) refers to a sequencing the bases of a small fragment of DNA are sequentially identified from signals emitted as each fragment is re-synthesized from a DNA template strand. NGS extends this process across millions of reactions in a massively parallel fashion, rather than being limited to a single or a few DNA fragments. This advance enables rapid sequencing of the amplified DNA, with the latest instruments capable of producing hundreds of gigabases of data in a single sequencing run. See, e.g., Shendure and Ji, Nature Biotechnology 26, 1135-1145 (2008) or Mardis, Annu Rev Genomics Hum Genet. 2008; 9:387-402. Suitable NGS platforms are available commercially, e.g. the Roche 454 platform, the Roche 454 Junior platform, the Illumina HiSeq or MiSeq platforms, or the Life Technologies SOLiD 5500 or Ion Torrent platforms.

Generally, a quantification (e.g. determining the amount of methylated target DNA) may be absolute, e.g. in pg per mL or ng per mL sample, copies per mL sample, number of PCR cycles etc., or it may be relative, e.g. 10 fold higher than in a control sample or as percentage of methylation of a reference control (preferably fully methylated DNA). Determining the amount of methylated target DNA in the sample may comprise normalizing for the amount of total DNA in the sample. Normalizing for the amount of total DNA in the test sample preferably comprises calculating the ratio of the amount of methylated target DNA and (i) the amount of DNA of a reference site or (ii) the amount of total DNA of the target (e.g. the amount of methylated target DNA plus the amount of unmethylated target DNA, the latter preferably measured on the reverse strand). A reference site can be any genomic site and does not have to be a gene. It is preferred that the number of occurrences of the sequence of the reference site is stable or expected to be stable (i.e. constant) over a large population (e.g. is not in a repeat, i.e. in repetitive DNA). The reference site can, for instance be a housekeeping gene such as beta-Actin.

As mentioned above, the amount of methylated target DNA in the sample may be expressed as the proportion of the amount of methylated target DNA relative to the amount of methylated target DNA (reference control) in a reference sample comprising substantially fully methylated genomic DNA. Preferably, determining the proportion of methylated target DNA comprises determining the amount of methylated DNA of the same target in a reference sample, inter sample normalization of total methylated DNA, preferably by using the methylation unspecific measurement of a reference site, and dividing the ratio derived from the test sample by the corresponding ratio derived from the reference sample. The proportion can be expressed as a percentage or PMR (Percentage of Methylated Reference) by multiplying the result of the division by 100. The determination of the PMR is described in detail in Ogino et al. (JMD May 2006, Vol. 8, No. 2).

The term “amplifying” or “generating an amplicon” as used herein refers to an increase in the number of copies of the target nucleic acid and its complementary sequence, or particularly a region thereof. The target can be a double-stranded or single-stranded DNA template. The amplification may be performed by using any method known in the art, typically with a polymerase chain reaction (PCR). An “amplicon” is a double-stranded fragment of DNA according to said defined region. The amplification is preferably performed by methylation-specific PCR (i.e. an amplicon is produced depending on whether one or more CpG sites are converted or not) using (i) methylation-specific primers, or (ii) primers which are methylation-unspecific, but specific to bisulfite-converted DNA (i.e. hybridize only to converted DNA by covering at least one converted C not in a CpG context). Methylation-specificity with (ii) is achieved by using methylation-specific blocker oligonucleotides, which hybridize specifically to converted or non-converted CpG sites and thereby terminate the PCR polymerization. For example, the step of amplifying comprises a real-time PCR, in particular HeavyMethyl™ or HeavyMethyl™-MethyLight™.

The term “genomic DNA” as used herein refers to chromosomal DNA and is used to distinguish from coding DNA. As such, it includes exons, introns as well as regulatory sequences, in particular promoters, belonging to a gene.

The phrase “converting, in DNA, cytosine unmethylated in the 5-position to uracil or another base that does not hybridize to guanine” as used herein refers to a process of chemically treating the DNA in such a way that all or substantially all of the unmethylated cytosine bases are converted to uracil bases, or another base which is dissimilar to cytosine in terms of base pairing behaviour, while the 5-methylcytosine bases remain unchanged. The conversion of unmethylated, but not methylated, cytosine bases within the DNA sample is conducted with a converting agent. The term “converting agent” as used herein relates to a reagent capable of converting an unmethylated cytosine to uracil or to another base that is detectably dissimilar to cytosine in terms of hybridization properties. The converting agent is preferably a bisulfite such as disulfite, or hydrogen sulfite. The reaction is performed according to standard procedures (Frommer et al., 1992, Proc Natl Acad Sci USA 89:1827-31; Olek, 1996, Nucleic Acids Res 24:5064-6; EP 1394172). It is also possible to conduct the conversion enzymatically, e.g by use of methylation specific cytidine deaminases. Most preferably, the converting agent is sodium bisulfite, ammonium bisulfite or bisulfite.

The term “bisulfite-specific” means specific for bisulfite-converted DNA. Bisulfite-converted DNA is DNA in which at least one C not in a CpG context (e.g. of a CpC, CpA or CpT dinucleotide), preferably all, has/have been converted into a T or U (chemically converted into U, which by DNA amplification becomes T). With respect to an oligonucleotide, it means that the oligonucleotide covers or hybridizes to at least one nucleotide derived from conversion of a C not in a CpG context (e.g. of a CpC, CpA or CpT dinucleotide) or its complement into a T.

The term “methylation-specific” as used herein refers generally to the dependency from the presence or absence of CpG methylation.

The term “methylation-specific” as used herein with respect to an oligonucleotide means that the oligonucleotide does or does not anneal to a single-strand of DNA (in which cytosine unmethylated in the 5-position has been converted to uracil or another base that does not hybridize to guanine, and where it comprises at least one CpG site before conversion) without a mismatch regarding the position of the C in the at least one CpG site, depending on whether the C of the at least one CpG sites was unmethylated or methylated prior to the conversion, i.e. on whether the C has been converted or not. The methylation-specificity can be either positive (the oligonucleotide anneals without said mismatch if the C was not converted) or negative (the oligonucleotide anneals without said mismatch if the C was converted). To prevent annealing of the oligonucleotide contrary to its specificity, it preferably covers at least 2, 3, 4, 5 or 6 and preferably 3 to 6 CpG sites before conversion.

The term “methylation-unspecific” as used herein refers generally to the independency from the presence or absence of CpG methylation.

The term “methylation-unspecific” as used herein with respect to an oligonucleotide means that the oligonucleotide does anneal to a single-strand of DNA (in which cytosine unmethylated in the 5-position has been converted to uracil or another base that does not hybridize to guanine, and where it may or may not comprise at least one CpG site before conversion) irrespective of whether the C of the at least one CpG site was unmethylated or methylated prior to the conversion, i.e. of whether the C has been converted or not. In one case, the region of the single-strand of DNA the oligonucleotide anneals to does not comprise any CpG sites (before and after conversion) and the oligonuclotide is methylation-unspecific solely for this reason. While a methylation-unspecific oligonucleotide may cover one or more CpG dinucleotides, it does so with mismatches and/or spacers. The term “mismatch” as used herein refers to base-pair mismatch in DNA, more specifically a base-pair that is unable to form normal base-pairing interactions (i.e., other than “A” with “T” or “U”, or “G” with “C”).

Methylation is detected within the at least one genomic DNA polynucleotide, i.e. in a particular region of the DNA according to the SEQ ID NO. referred to (the “target DNA”). The term “target DNA” as used herein refers to a genomic nucleotide sequence at a specific chromosomal location. In the context of the present invention, it is typically a genetic marker that is known to be methylated in the state of disease (for example in cancer cells vs. non-cancer cells). A genetic marker can be a coding or non-coding region of genomic DNA.

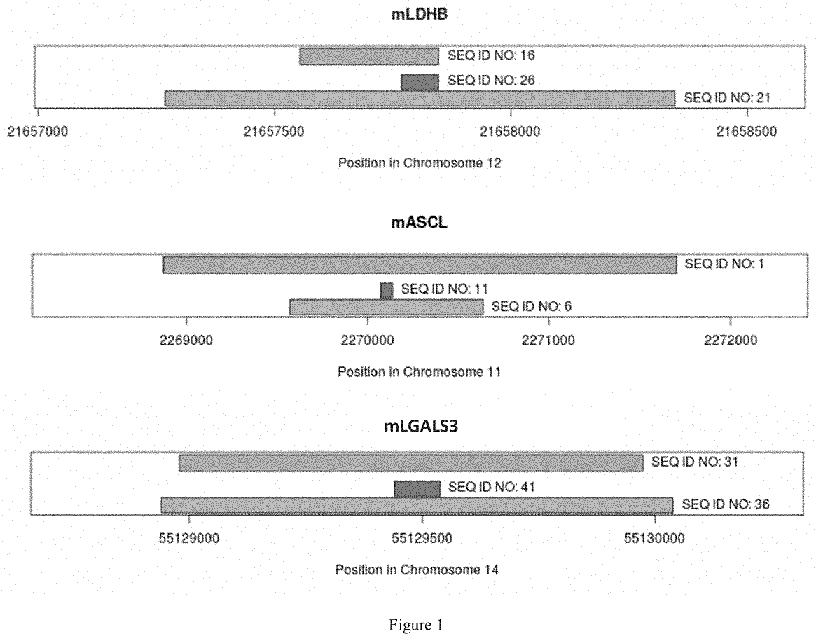

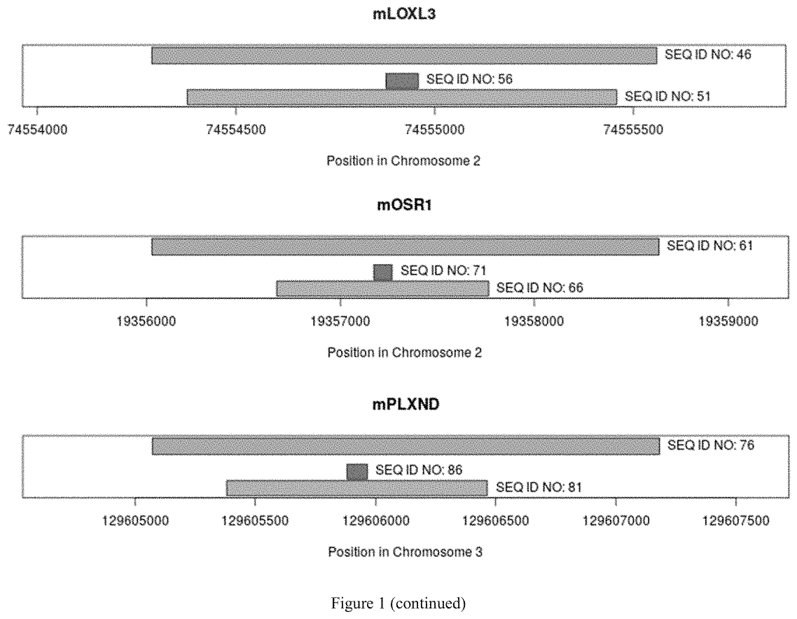

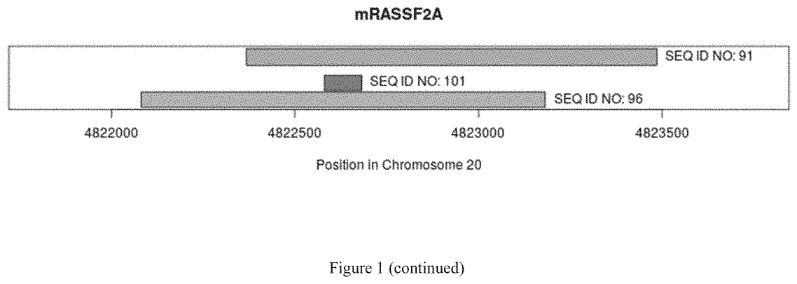

The term “region of the target DNA” or “region of the converted DNA” as used herein refers to a part of the target DNA which is to be analysed. Preferably, the region is at least 40, 50, 60, 70, 80, 90, 100, 150, or 200 or 300 base pairs (bp) long and/or not longer than 500, 600, 700, 800, 900 or 1000 bp (e.g. 25-500, 50-250 or 75-150 bp). In particular, it is a region comprising at least 3, 4, 5, 6, 7, 8, 9, 10, 11, 12, 13, 14, 15 or 16 CpG sites of the genomic DNA. The target DNAs of the invention are given in FIG. 1 and Table 3.

For an amplification of the target region with at least one methylation-specific primer, it is preferred that the at least one methylation-specific primer covers at least 1, at least 2 or preferably at least 3 CpG sites (e.g. 2-8 or preferably 3-6 CpG sites) of the target region. Preferably, at least 1, at least 2 or preferably at least 3 CpG sites of these CpG sites are covered by the 3' third of the primer (and/or one of these CpG sites is covered by the 3′ end of the primer (last three nucleotides of the primer).

The term “covering a CpG site” as used herein with respect to an oligonucleotide refers to the oligonucleotide annealing to a region of DNA comprising this CpG site, before or after conversion of the C of the CpG site (i.e. the CpG site of the corresponding genomic DNA when it is referred to a bisulfite converted sequence). The annealing may, with respect to the CpG site (or former CpG site if the C was converted), be methylation-specific or methylation-unspecific as described herein.

The term “annealing”, when used with respect to an oligonucleotide, is to be understood as a bond of an oligonucleotide to an at least substantially complementary sequence along the lines of the Watson-Crick base pairings in the sample DNA, forming a duplex structure, under moderate or stringent hybridization conditions. When it is used with respect to a single nucleotide or base, it refers to the binding according to Watson-Crick base pairings, e.g. C-G, A-T and A-U. Stringent hybridization conditions involve hybridizing at 68° C. in 5×SSC/5×Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderate conditions involve washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The cancer of the specification includes the following stages (as defined by the corresponding TNM classification(s) in brackets) of the cancer and each of its subtypes: stage 0 (T is, N0, M0), stage I (T1, N0, M0), stage II (T2, N0, M0), stage III (T3, N0, M0; or T1 to T3, N1, M0), stage IVA (T4a, N0 or N1, M0; or T1 to T4a, N2, M0), stage IVB (T4b, any N, MO or any T, N3, M0), and stage IVC (any T, any N, M1). The TNM classification is a staging system for malignant cancer. As used herein the term “TNM classification” refers to the 6th edition of the TNM stage grouping as defined in Sobin et al. (International Union Against Cancer (UICC), TNM Classification of Malignant tumors, 6th ed. New York; Springer, 2002, pp. 191-203).

The term “subject” as used herein refers to a human individual.

The term “biological sample” as used herein refers to material obtained from a subject and comprises genomic DNA from all chromosomes, preferably genomic DNA covering the whole genome. Preferably, the sample comprises cell-free genomic DNA (including the target DNA), preferably circulating genomic DNA. If a subject has cancer, the cell-free (preferably circulating) genomic DNA comprises cell-free (preferably circulating) genomic DNA from cancer cells, i.e. preferably ctDNA.

The term “liquid biopsy” as used herein refers to a body fluid sample comprising cell-free (preferably circulating) genomic DNA. It is envisaged that it is a body liquid in which cell-free (preferably circulating) genomic DNA from cells of the cancer of the specification can be found if the subject has the cancer. A “blood-derived sample” is any sample that is derived by in vitro processing from blood, e.g. plasma or serum. “A sample comprising cell-free DNA from blood” can be any such sample. For example, urine comprises cell-free DNA from blood.

The term “cell-free DNA” as used herein or its synonyms “cfDNA”, and “extracellular DNA”, “circulating DNA” and “free circulating DNA” refers to DNA that is not comprised within an intact cell in the respective body fluid which is the sample or from which the sample is derived, but which is free in the body liquid sample. Cell-free DNA usually is genomic DNA that is fragmented as described below.

The term “circulating DNA” or “free circulating DNA” as used herein refers to cell-free DNA in a body liquid (in particular blood) which circulates in the body.

The term “circulating tumor DNA” or “ctDNA” as used herein refers to circulating DNA that is derived from a tumor (i.e. cell-free DNA derived from tumor cells).

Typically, in samples comprising the target DNA, especially extracellular target DNA, from cancer cells, there is also target DNA from non-cancer cells which is not methylated contrary to the target DNA from cancer cells. Usually, said target DNA from non-cancer cells exceeds the amount from diseased cells by at least 10-fold, at least 100-fold, at least 1,000-fold or at least 10,000-fold. Generally, the genomic DNA comprised in the sample is at least partially fragmented. “At least partially fragmented” means that at least the extracellular DNA, in particular at least the extracellular target DNA, from cancer cells, is fragmented. The term “fragmented genomic DNA” refers to pieces of DNA of the genome of a cell, in particular a cancer cell, that are the result of a partial physical, chemical and/or biological break-up of the lengthy DNA into discrete fragments of shorter length. Particularly, “fragmented” means fragmentation of at least some of the genomic DNA, preferably the target DNA, into fragments shorter than 1,500 bp, 1,300 bp, 1,100 bp, 1,000 bp, 900 bp, 800 bp, 700 bp, 600 bp, 500 bp, 400 bp, 300 bp, 200 bp or 100 bp. “At least some” in this respect means at least 5%, 10%, 20%, 30%, 40%, 50% or 75%.

The term “cancer cell” as used herein refers to a cell that acquires a characteristic set of functional capabilities during their development, particularly one or more of the following: the ability to evade apoptosis, self-sufficiency in growth signals, insensitivity to anti-growth signals, tissue invasion/metastasis, significant growth potential, and/or sustained angiogenesis. The term is meant to encompass both pre-malignant and malignant cancer cells.

The term “a significant amount of methylated genomic DNA” as used herein refers to an amount of at least X molecules of the methylated target DNA per ml of the sample used, preferably per ml of blood, serum or plasma. X may be as low as 1 and is usually a value between and including 1 and 50, in particular at least 2, 3, 4, 5, 10, 15, 20, 25, 30 or 40. For determination whether there is such a significant amount, the methylated target DNA may be, but does not necessarily have to be quantified. The determination, if no quantification is performed, may also be made by comparison to a standard, for example a standard comprising genomic DNA and therein a certain amount of fully methylated DNA, e.g. the equivalence of X genomes, wherein X is as above. The term may also refer to an amount of at least Y % of methylated target DNA in the sample (wherein the sum of methylated and unmethylated target DNA is 100%), wherein Y may be as low as 0.05 and is usually a value between and including 0.05 and 5, preferably 0.05 and 1 and more preferably 0.05 and 0.5. For example, Y may be at least 0.05, 0.1, 0.2, 0.3, 0.5, 1.0, 1.5, 2.0, 2.5, 3.0, 4.0 or 5.0.

The term “tumor DNA” or “tumor DNA of a cancer cell” as used herein refers simply to DNA of a cancer cell. It is used only to distinguish DNA of a cancer cell more clearly from other DNA referred to herein. Thus, unless ambiguities are introduced, the term “DNA of a cancer cell” may be used instead.

The term “is indicative for” or “indicates” as used herein refers to an act of identifying or specifying the thing to be indicated. As will be understood by persons skilled in the art, such assessment normally may not be correct for 100% of the subjects, although it preferably is correct. The term, however, requires that a correct indication can be made for a statistically significant part of the subjects. Whether a part is statistically significant can be determined easily by the person skilled in the art using several well-known statistical evaluation tools, for example, determination of confidence intervals, determination of p values, Student's t-test, Mann-Whitney test, etc. Details are provided in Dowdy and Wearden, Statistics for Research, John Wiley & Sons, New York 1983. The preferred confidence intervals are at least 50%, at least 60%, at least 70%, at least 80%, at least 90%, at least 95%. The p values are preferably 0.05, 0.01, or 0.005.

The phrase “method for detecting the presence or absence” as used herein with regard to the cancer of the specification refers to a determination whether the subject has the cancer or not. As will be understood by persons skilled in the art, such assessment normally may not be correct for 100% of the subjects, although it preferably is correct. The term, however, requires that a correct indication can be made for a statistically significant part of the subjects. For a description of statistic significance and suitable confidence intervals and p values, see above.

The term “diagnosis” as used herein refers to a determination whether a subject does or does not have cancer. A diagnosis by methylation analysis of the target DNA as described herein may be supplemented with a further means as described herein to confirm the cancer detected with the methylation analysis. As will be understood by persons skilled in the art, the diagnosis normally may not be correct for 100% of the subjects, although it preferably is correct. The term, however, requires that a correct diagnosis can be made for a statistically significant part of the subjects. For a description of statistic significance and suitable confidence intervals and p values, see above.

The phrase “screening a population of subjects” as used herein with regard to the cancer of the specification refers to the use of the method of the first aspect with samples of a population of subjects. Preferably, the subjects have an increased risk for, are suspected of having, or have had the cancer. In particular, one or more of the risk factors recited herein can be attributed to the subjects of the population. In a specific embodiment, the same one or more risk factors can be attributed to all subjects of the population. For example, the population may consist of subjects characterized by one or more risk factors described herein. It is to be understood that the term “screening” refers to a diagnosis as described above for subjects of the population, and is preferably confirmed using a further means as described herein. As will be understood by persons skilled in the art, the screening result normally may not be correct for 100% of the subjects, although it preferably is correct. The term, however, requires that a correct screening result can be achieved for a statistically significant part of the subjects. For a description of statistic significance and suitable confidence intervals and p values, see above.

The term “monitoring” as used herein refers to the accompaniment of a diagnosed cancer during a treatment procedure or during a certain period of time, typically during at least 1 week, 2 weeks, 3 weeks, 4 weeks, 1 month, 2 months, 3 months, 4 months, 5 months, 6 months, 1 year, 2 years, 3 years, 5 years, 10 years, or any other period of time. The term “accompaniment” means that states of and, in particular, changes of these states of a cancer may be detected based on the amount of methylated target DNA, particular based on changes in the amount in any type of periodical time segment, determined e.g., daily or 1, 2, 3, 4, 5, 6, 7, 8, 9, 10, 11, 12, 13, 14 or 15 times per month (no more than one determination per day) over the course of the treatment, which may be up to 1, 2, 3, 4, 5, 6, 7, 8, 9, 10, 11, 12, 15 or 24 months. Amounts or changes in the amounts can also be determined at treatment specific events, e.g. before and/or after every treatment cycle or drug/therapy administration. A cycle is the time between one round of treatment until the start of the next round. Cancer treatment is usually not a single treatment, but a course of treatments. A course usually takes between 3 to 6 months, but can be more or less than that. During a course of treatment, there are usually between 4 to 8 cycles of treatment. Usually a cycle of treatment includes a treatment break to allow the body to recover. As will be understood by persons skilled in the art, the result of the monitoring normally may not be correct for 100% of the subjects, although it preferably is correct. The term, however, requires that a correct result of the monitoring can be achieved for a statistically significant part of the subjects. For a description of statistic significance and suitable confidence intervals and p values, see above.

“Substantially identical” means that an oligonucleotide does not need to be 100% identical to a reference sequence but can comprise mismatches and/or spacers as defined herein. It is preferred that a substantially identical oligonucleotide, if not 100% identical, comprises 1 to 3, i.e. 1, 2 or 3 mismatches and/or spacers, preferably one mismatch or spacer per oligonucleotide, such that the intended annealing does not fail due to the mismatches and/or spacers. To enable annealing despite mismatches and/or spacers, it is preferred that an oligonucleotide does not comprise more than 1 mismatch per 10 nucleotides (rounded up if the first decimal is 5 or higher, otherwise rounded down) of the oligonucleotide.

The mismatch or a spacer is preferably a mismatch with or a spacer covering an SNP in the genomic DNA of the subject. A mismatch with an SNP is preferably not complementary to any nucleotide at this position in the subject's species. The term “SNP” as used herein refers to the site of an SNP, i.e. a single nucleotide polymorphism, at a particular position in the (preferably human) genome that varies among a population of individuals. SNPs of the genomic DNA the present application refers to are known in the art and can be found in online databases such as dbSNP of NCBI (http://www.ncbi.nlm.nih.gov/snp).

The term “spacer” as used herein refers to a non-nucleotide spacer molecule, which increases, when joining two nucleotides, the distance between the two nucleotides to about the distance of one nucleotide (i.e. the distance the two nucleotides would be apart if they were joined by a third nucleotide). Non-limiting examples for spacers are Inosine, d-Uracil, halogenated bases, Amino-dT, C3, C12, Spacer 9, Spacer 18, and dSpacer.

The term “oligonucleotide” as used herein refers to a linear oligomer of 5 to 50 ribonucleotides or preferably deoxyribonucleotides. Preferably, it has the structure of a single-stranded DNA fragment. The “stretch of contiguous nucleotides” referred to herein preferably is as long as the oligonucleotide.

The term “primer oligonucleotide” as used herein refers to a single-stranded oligonucleotide sequence comprising at its 3′ end a priming region which is substantially complementary to a nucleic acid sequence sought to be copied (the template) and serves as a starting point for synthesis of a primer extension product. Preferably, the priming region is 10 to 40 nucleotides, more preferably 15-30 nucleotides and most preferably 19 to 25 nucleotides in length. The “stretch of contiguous nucleotides” referred to herein preferably corresponds to the priming region. The primer oligonucleotide may further comprise, at the 5′ end of the primer oligonucleotide, an overhang region. The overhang region consists of a sequence which is not complementary to the original template, but which is in a subsequent amplification cycle incorporated into the template by extension of the opposite strand. The overhang region has a length that does not prevent priming by the priming region (e.g. annealing of the primer via the priming region to the template). For example, it may be 1-200 nucleotides, preferably 4-100 or 4-50, more preferably 4-25 or most preferably 4-15 nucleotides long. The overhang region usually comprises one or more functional domains, i.e. it has a sequence which encodes (not in the sense of translation into a polypeptide) a function which is or can be used for the method of the first aspect. Examples of functional domains are restriction sites, ligation sites, universal priming sites (e.g. for NGS), annealing sites (not for annealing to the template to be amplified by extension of the priming region, but to other oligonucleotides), and index (barcode) sites. The overhang region does not comprise a “stretch of contiguous nucleotides” as referred to herein with respect to the methylation markers of the invention. It is, as indicated above, understood by the skilled person that the sequence of an overhang region incorporated into a new double-strand generated by amplification. Therefore, the overhang region could be considered part of the priming region for further amplification of the new double-strand. However, the term “priming region” is used herein to distinguish a region that is the priming region of the initial template, i.e. which has a sequence that substantially corresponds to a methylation marker sequence of Table 3, from an overhang region with respect to the same methylation marker sequence.

It is also understood by the skilled person that the term “template” in the context of amplification of bisulfite converted DNA comprises not only double-stranded DNA, but also a single strand that is the result of bisulfite conversion of genomic DNA (rendering it non-complementary to its previous opposite strand). In the first round of amplification, only one of the primers of a primer pair binds to this single-strand and is extended, thereby creating a new complementary opposite strand to which the other primer of the primer pair can bind. Table 3 provides the sequences of the strands that are the result of bisulfite conversion of the genomic DNA of the methylation markers of the invention (bis1 and bis2), as well as corresponding new complementary opposite strands in 5′-3′ orientation (rc).

The term “primer pair” as used herein refers to two oligonucleotides, namely a forward and a reverse primer, that have, with respect to a double-stranded nucleic acid molecule (including a single strand that is the result of bisulfite conversion plus the new complementary opposite strand to be created as explained above), sequences that are (at least substantially) identical to one strand each such that they each anneal to the complementary strand of the strand they are (at least substantially) identical to. The term “forward primer” refers to the primer which is (at least substantially) identical to the forward strand (as defined by the direction of the genomic reference sequence) of the double-stranded nucleic acid molecule, and the term “reverse primer” refers to the primer which is (at least substantially) identical to the reverse complementary strand of the forward strand in the double-stranded nucleic acid molecule. The distance between the sites where forward and reverse primer anneal to their template depends on the length of the amplicon the primers are supposed to allow generating. Typically, with respect to the present invention it is between 40 and 1000 bp. Preferred amplicon sizes are specified herein. In case of single-stranded DNA template that is to be amplified using a pair of primers, only one of the primers anneals to the single strand in the first amplification cycle. The other primer then binds to the newly generated complementary strand such that the result of amplification is a double-stranded DNA fragment.

The term “blocker” as used herein refers to a molecule which binds in a methylation-specific manner to a single-strand of DNA (i.e. it is specific for either the converted methylated or preferably for the converted unmethylated DNA or the amplified DNA derived from it) and prevents amplification of the DNA by binding to it, for example by preventing a primer to bind or by preventing primer extension where it binds. Non-limiting examples for blockers are sequence and/or methylation specific antibodies (blocking e.g. primer binding or the polymerase) and in particular blocker oligonucleotides.

A “blocker oligonucleotide” may be a blocker that prevents the extension of the primer located upstream of the blocker oligonucleotide. It comprises nucleosides/nucleotides having a backbone resistant to the 5′ nuclease activity of the polymerase. This may be achieved, for example, by comprising peptide nucleic acid (PNA), locked nucleic acid (LNA), Morpholino, glycol nucleic acid (GNA), threose nucleic acid (TNA), bridged nucleic acids (BNA), N3′-P5′ phosphoramidate (NP) oligomers, minor groove binder-linked-oligonucleotides (MGB-linked oligonucleotides), phosphorothioate (PS) oligomers, CrC₄alkylphosphonate oligomers, phosphoramidates, β-phosphodiester oligonucleotides, a-phosphodiester oligonucleotides or a combination thereof. Alternatively, it may be a non-extendable oligonucleotide with a binding site on the DNA single-strand that overlaps with the binding site of a primer oligonucleotide. When the blocker is bound, the primer cannot bind and therefore the amplicon is not generated. When the blocker is not bound, the primer-binding site is accessible and the amplicon is generated. For such an overlapping blocker, it is preferable that the affinity of the blocker is higher than the affinity of the primer for the DNA. A blocker oligonucleotide is typically 15 to 50, preferably 20 to 40 and more preferably 25 to 35 nucleotides long. “At least one blocker” refers in particular to a number of 1, 2, 3, 4 or 5 blockers, more particularly to 1-2 or 1-3 blockers. Also, a blocker oligonucleotide cannot by itself act as a primer (i.e. cannot be extended by a polymerase) due to a non-extensible 3′ end.

The term “probe oligonucleotide” or “probe” as used herein refers to an oligonucleotide that is used to detect an amplicon by annealing to one strand of the amplicon, usually not where any of the primer oligonucleotides binds (i.e. not to a sequence segment of the one strand which overlaps with a sequence segment a primer oligonucleotide anneals to). Preferably it anneals without a mismatch or spacer, in other words it is preferably complementary to one strand of the amplicon. A probe oligonucleotide is preferably 5-40 nucleotides, more preferably 10 to 25 and most preferably 15 to 20 nucleotides long. The “stretch of contiguous nucleotides” referred to herein preferably is as long as the probe oligonucleotide. Usually, the probe is linked, preferably covalently linked, to at least one detectable label which allows detection of the amplicon and/or at least one quencher which allows quenching the signal of a (preferably the) detectable label. The term “detectable label” as used herein does not exhibit any particular limitation. The detectable label may be selected from the group consisting of radioactive labels, luminescent labels, fluorescent dyes, compounds having an enzymatic activity, magnetic labels, antigens, and compounds having a high binding affinity for a detectable label. For example, fluorescent dyes linked to a probe may serve as a detection label, e.g. in a real-time PCR. Suitable radioactive markers are P-32, S-35, 1-125, and H-3, suitable luminescent markers are chemiluminescent compounds, preferably luminol, and suitable fluorescent markers are preferably dansyl chloride, fluorcein-5-isothiocyanate, and 4-fluor-7-nitrobenz-2-aza-1,3 diazole, in particular 6-Carboxyfluorescein (FAM), 6-Hexachlorofluorescein (HEX), 5(6)-Carboxytetramethylrhodamine (TAMRA), 5(6)-Carboxy-X-Rhodamine (ROX), Cyanin-5-Fluorophor (Cy5) and derivates thereof; suitable enzyme markers are horseradish peroxidase, alkaline phosphatase, a-galactosidase, acetylcholinesterase, or biotin. A probe may also be linked to a quencher. The term “quencher” as used herein refers to a molecule that deactivates or modulates the signal of a corresponding detectable label, e.g. by energy transfer, electron transfer, or by a chemical mechanism as defined by IUPAC (see compendium of chemical terminology 2^{nd} ed. 1997). In particular, the quencher modulates the light emission of a detectable label that is a fluorescent dye. In some cases, a quencher may itself be a fluorescent molecule that emits fluorescence at a characteristic wavelength distinct from the label whose fluorescence it is quenching. In other cases, the quencher does not itself fluoresce (i.e., the quencher is a “dark acceptor”). Such quenchers include, for example, dabcyl, methyl red, the QSY diarylrhodamine dyes, and the like.

The term “treatment” or “treating” with respect to cancer as used herein refers to a therapeutic treatment, wherein the goal is to reduce progression of cancer. Beneficial or desired clinical results include, but are not limited to, release of symptoms, reduction of the length of the disease, stabilized pathological state (specifically not deteriorated), slowing down of the disease's progression, improving the pathological state and/or remission (both partial and total), preferably detectable. A successful treatment does not necessarily mean cure, but it can also mean a prolonged survival, compared to the expected survival if the treatment is not applied. In a preferred embodiment, the treatment is a first line treatment, i.e. the cancer was not treated previously. Cancer treatment involves a treatment regimen.

The term “treatment regimen” as used herein refers to how the subject is treated in view of the disease and available procedures and medication. Non-limiting examples of cancer treatment regimens are chemotherapy, surgery and/or irradiation or combinations thereof. The early detection of cancer the present invention enables allows in particular for a surgical treatment, especially for a curative resection. In particular, the term “treatment regimen” refers to administering one or more anti-cancer agents or therapies as defined below. The term “anti-cancer agent or therapy” as used herein refers to chemical, physical or biological agents or therapies, or surgery, including combinations thereof, with antiproliferative, antioncogenic and/or carcinostatic properties.

A chemical anti-cancer agent or therapy may be selected from the group consisting of alkylating agents, antimetabolites, plant alkaloyds and terpenoids and topoisomerase inhibitors. Preferably, the alkylating agents are platinum-based compounds. In one embodiment, the platinum-based compounds are selected from the group consisting of cisplatin, oxaliplatin, eptaplatin, lobaplatin, nedaplatin, carboplatin, iproplatin, tetraplatin, lobaplatin, DCP, PLD-147, JM1 18, JM216, JM335, and satraplatin.

A physical anti-cancer agent or therapy may be selected from the group consisting of radiation therapy (e.g. curative radiotherapy, adjuvant radiotherapy, palliative radiotherapy, teleradiotherapy, brachytherapy or metabolic radiotherapy), phototherapy (using, e.g. hematoporphoryn or photofrin II), and hyperthermia.

Surgery may be a curative resection, palliative surgery, preventive surgery or cytoreductive surgery. Typically, it involves an excision, e.g. intracapsular excision, marginal, extensive excision or radical excision as described in Baron and Valin (Rec. Med. Vet, Special Canc. 1990; 11(166):999-1007).

A biological anti-cancer agent or therapy may be selected from the group consisting of antibodies (e.g. antibodies stimulating an immune response destroying cancer cells such as retuximab or alemtuzubab, antibodies stimulating an immune response by binding to receptors of immune cells an inhibiting signals that prevent the immune cell to attack “own” cells, such as ipilimumab, antibodies interfering with the action of proteins necessary for tumor growth such as bevacizumab, cetuximab or panitumumab, or antibodies conjugated to a drug, preferably a cell-killing substance like a toxin, chemotherapeutic or radioactive molecule, such as Y-ibritumomab tiuxetan, I-tositumomab or ado-trastuzumab emtansine), cytokines (e.g. interferons or interleukins such as INF-alpha and IL-2), vaccines (e.g. vaccines comprising cancer-associated antigens, such as sipuleucel-T), oncolytic viruses (e.g. naturally oncolytic viruses such as reovirus, Newcastle disease virus or mumps virus, or viruses genetically engineered viruses such as measles virus, adenovirus, vaccinia virus or herpes virus preferentially targeting cells carrying cancer-associated antigens), gene therapy agents (e.g. DNA or RNA replacing an altered tumor suppressor, blocking the expression of an oncogene, improving a subject's immune system, making cancer cells more sensitive to chemotherapy, radiotherapy or other treatments, inducing cellular suicide or conferring an anti-angiogenic effect) and adoptive T cells (e.g. subject-harvested tumor-invading T-cells selected for antitumor activity, or subject-harvested T-cells genetically modified to recognize a cancer-associated antigen).

In one embodiment, the one or more anti-cancer drugs is/are selected from the group consisting of Abiraterone Acetate, ABVD, ABVE, ABVE-PC, AC, AC-T, ADE, Ado-Trastuzumab Emtansine, Afatinib Dimaleate, Aldesleukin, Alemtuzumab, Aminolevulinic Acid, Anastrozole, Aprepitant, Arsenic Trioxide, Asparaginase Erwinia chrysanthemi, Axitinib, Azacitidine, BEACOPP, Belinostat, Bendamustine Hydrochloride, BEP, Bevacizumab, Bexarotene, Bicalutamide, Bleomycin, Bortezomib, Bosutinib, Brentuximab Vedotin, Busulfan, Cabazitaxel, Cabozantinib-S-Malate, CAFCapecitabine, CAPDX, Carboplatin, CARBOPLATIN-TAXOL, Carfilzomib, Carmustine, Carmustine Implant, Ceritinib, Cetuximab, Chlorambucil, CHLORAMBUCIL-PREDNISONE, CHOP, Cisplatin, Clofarabine, CMF, COPP, COPP-ABV, Crizotinib, CVP, Cyclophosphamide, Cytarabine, Cytarabine, Liposomal, Dabrafenib, Dacarbazine, Dactinomycin, Dasatinib, Daunorubicin Hydrochloride, Decitabine, Degarelix, Denileukin Diftitox, Denosumab, Dexrazoxane Hydrochloride, Docetaxel, Doxorubicin Hydrochloride, Doxorubicin Hydrochloride Liposome, Eltrombopag Olamine, Enzalutamide, Epirubicin Hydrochloride, EPOCH, Eribulin Mesylate, Erlotinib Hydrochloride, Etoposide Phosphate, Everolimus, Exemestane, FEC, Filgrastim, Fludarabine Phosphate, Fluorouracil, FU-LV, Fulvestrant, Gefitinib, Gemcitabine Hydrochloride, GEMCITABINE-CISPLATIN, GEMCITABINE-OXALIPLATIN, Gemtuzumab Ozogamicin, Glucarpidase, Goserelin Acetate, HPV Bivalent Vaccine, Recombinant HPV Quadrivalent Vaccine, Hyper-CVAD, Ibritumomab Tiuxetan, Ibrutinib, ICE, Idelalisib, Ifosfamide, Imatinib, Mesylate, Imiquimod, Iodine 131 Tositumomab and Tositumomab, Ipilimumab, Irinotecan Hydrochloride, Ixabepilone, Lapatinib Ditosylate, Lenalidomide, Letrozole, Leucovorin Calcium, Leuprolide Acetate, Liposomal Cytarabine, Lomustine, Mechlorethamine Hydrochloride, Megestrol Acetate, Mercaptopurine, Mesna, Methotrexate, Mitomycin C, Mitoxantrone Hydrochloride, MOPP, Nelarabine, Nilotinib, Obinutuzumab, Ofatumumab, Omacetaxine Mepesuccinate, OEPA, OFF, OPPA, Oxaliplatin, Paclitaxel, Paclitaxel Albumin-stabilized Nanoparticle Formulation, PAD, Palifermin, Palonosetron Hydrochloride, Pamidronate Disodium, Panitumumab, Pazopanib Hydrochloride, Pegaspargase, Peginterferon Alfa-2b, Pembrolizumab, Pemetrexed Disodium, Pertuzumab, Plerixafor, Pomalidomide, Ponatinib Hydrochloride, Pralatrexate, Prednisone, Procarbazine Hydrochloride, Radium 223 Dichloride, Raloxifene Hydrochloride, Ramucirumab, Rasburicase, R—CHOP, R—CVP, Recombinant HPV Bivalent Vaccine, Recombinant HPV Quadrivalent Vaccine, Recombinant Interferon Alfa-2b, Regorafenib, Rituximab, Romidepsin, Romiplostim, Ruxolitinib Phosphate, Siltuximab, Sipuleucel-T, Sorafenib Tosylate, STANFORD V, Sunitinib Malate, TAC, Talc, Tamoxifen Citrate, Temozolomide, Temsirolimus, Thalidomide, Topotecan Hydrochloride, Toremifene, Tositumomab and I 131 Iodine Tositumomab, TPF, Trametinib, Trastuzumab, Vandetanib, VAMP, VeIP, Vemurafenib, Vinblastine Sulfate, Vincristine Sulfate, Vincristine Sulfate Liposome, Vinorelbine Tartrate, Vismodegib, Vorinostat, XELOX, Ziv-Aflibercept, and Zoledronic Acid.

### SEQ IDs Referred to in the Application

The present application refers to SEQ ID NOs 1-119. An overview and explanation of these SED IDs is given in the following Table 3.

TABLE 3
SEQ ID NOs of the specification. m as first letter of the gene name
means methylated, rc means reverse complement, C to T or G to A means
converted by bisulfite conversion of cytosines outside of CpG context
into uracil and replaced by thymidine in subsequent amplification.
bis1 refers to the bisulfite converted forward strand (as recited
in the SEQ ID of the respective genomic DNA) and bis2 to the bisulfite
converted reverse complement strand of the forward strand (reverse
complement of the SEQ ID of the respective genomic DNA), whereby
the direction of the strand is defined by the direction of the genomic
reference sequence as e.g. obtained from the genome build (GRCh38).
For a mapping of the sequences, see FIG. 1.
mASCL2 Assay + CpG island 11: 2268874-2271702

| | |
|---|---|
| SEQ ID NO: 1 | genomic reference |
| SEQ ID NO: 2 | C to T (bis1) |
| SEQ ID NO: 3 | rc C to T (bis1) |
| SEQ ID NO: 4 | G to A (bis2 rc) |
| SEQ ID NO: 5 | rc G to A (bis2 rc) |

mASCL2 Extended Assay 11: 2269571-2270636

| | |
|---|---|
| SEQ ID NO: 6 | genomic reference |
| SEQ ID NO: 7 | C to T (bis1) |
| SEQ ID NO: 8 | rc C to T (bis1) |
| SEQ ID NO: 9 | G to A (bis2 rc) |
| SEQ ID NO: 10 | rc G to A (bis2 rc) |

mASCL2 Assay 11: 2270071-2270136

| | |
|---|---|
| SEQ ID NO: 11 | genomic reference |
| SEQ ID NO: 12 | C to T (bis1) |
| SEQ ID NO: 13 | rc C to T (bis1) |
| SEQ ID NO: 14 | G to A (bis2 rc) |
| SEQ ID NO: 15 | rc G to A (bis2 rc) |

mLDHB Assay + CpG island 12: 21657554-21657847

| | |
|---|---|
| SEQ ID NO: 16 | genomic reference |
| SEQ ID NO: 17 | C to T (bis1) |
| SEQ ID NO: 18 | rc C to T (bis1) |
| SEQ ID NO: 19 | G to A (bis2 rc) |
| SEQ ID NO: 20 | rc G to A (bis2 rc) |

mLDHB Extended Assay 12: 21657268-21658347

| | |
|---|---|
| SEQ ID NO: 21 | genomic reference |
| SEQ ID NO: 22 | C to T (bis1) |
| SEQ ID NO: 23 | rc C to T (bis1) |
| SEQ ID NO: 24 | G to A (bis2 rc) |
| SEQ ID NO: 25 | rc G to A (bis2 rc) |

mLDHB Assay 12: 21657768-21657847

| | |
|---|---|
| SEQ ID NO: 26 | genomic reference |
| SEQ ID NO: 27 | C to T (bis1) |
| SEQ ID NO: 28 | rc C to T (bis1) |
| SEQ ID NO: 29 | G to A (bis2 rc) |
| SEQ ID NO: 30 | rc G to A (bis2 rc) |

mLGALS3 Assay + CpG island 14: 55128979-55129974

| | |
|---|---|
| SEQ ID NO: 31 | genomic reference |
| SEQ ID NO: 32 | C to T (bis1) |
| SEQ ID NO: 33 | rc C to T (bis1) |
| SEQ ID NO: 34 | G to A (bis2 rc) |
| SEQ ID NO: 35 | rc G to A (bis2 rc) |

mLGALS3 Extended Assay 14: 55128940-55130038

| | |
|---|---|
| SEQ ID NO: 36 | genomic reference |
| SEQ ID NO: 37 | C to T (bis1) |
| SEQ ID NO: 38 | rc C to T (bis1) |
| SEQ ID NO: 39 | G to A (bis2 rc) |
| SEQ ID NO: 40 | rc G to A (bis2 rc) |

mLGALS3 Assay 14: 55129440-55129538

| | |
|---|---|
| SEQ ID NO: 41 | genomic reference |
| SEQ ID NO: 42 | C to T (bis1) |
| SEQ ID NO: 43 | rc C to T (bis1) |
| SEQ ID NO: 44 | G to A (bis2 rc) |
| SEQ ID NO: 45 | rc G to A (bis2 rc) |

mLOXL3 Assay + CpG island 2: 74554288-74555558

| | |
|---|---|
| SEQ ID NO: 46 | genomic reference |
| SEQ ID NO: 47 | C to T (bis1) |
| SEQ ID NO: 48 | rc C to T (bis1) |
| SEQ ID NO: 49 | G to A (bis2 rc) |
| SEQ ID NO: 50 | rc G to A (bis2 rc) |

mLOXL3 Extended Assay 2: 74554377-74555458

| | |
|---|---|
| SEQ ID NO: 51 | genomic reference |
| SEQ ID NO: 52 | C to T (bis1) |
| SEQ ID NO: 53 | rc C to T (bis1) |
| SEQ ID NO: 54 | G to A (bis2 rc) |
| SEQ ID NO: 55 | rc G to A (bis2 rc) |

mLOXL3 Assay 2: 74554877-74554958

| | |
|---|---|
| SEQ ID NO: 56 | genomic reference |
| SEQ ID NO: 57 | C to T (bis1) |
| SEQ ID NO: 58 | rc C to T (bis1) |
| SEQ ID NO: 59 | G to A (bis2 rc) |
| SEQ ID NO: 60 | rc G to A (bis2 rc) |

mOSR1 Assay + CpG island 2: 19356028-19358642

| | |
|---|---|
| SEQ ID NO: 61 | genomic reference |
| SEQ ID NO: 62 | C to T (bis1) |
| SEQ ID NO: 63 | rc C to T (bis1) |
| SEQ ID NO: 64 | G to A (bis2 rc) |
| SEQ ID NO: 65 | rc G to A (bis2 rc) |

mOSR1 Extended Assay 2: 19356672-19357766

| | |
|---|---|
| SEQ ID NO: 66 | genomic reference |
| SEQ ID NO: 67 | C to T (bis1) |
| SEQ ID NO: 68 | rc C to T (bis1) |
| SEQ ID NO: 69 | G to A (bis2 rc) |
| SEQ ID NO: 70 | rc G to A (bis2 rc) |

mOSR1 Assay 2: 19357172-19357266

| | |
|---|---|
| SEQ ID NO: 71 | genomic reference |
| SEQ ID NO: 72 | C to T (bis1) |
| SEQ ID NO: 73 | rc C to T (bis1) |
| SEQ ID NO: 74 | G to A (bis2 rc) |
| SEQ ID NO: 75 | rc G to A (bis2 rc) |

mPLXND1 Assay + CpG island 3: 129605072-129607181

| | |
|---|---|
| SEQ ID NO: 76 | genomic reference |
| SEQ ID NO: 77 | C to T (bis1) |
| SEQ ID NO: 78 | rc C to T (bis1) |
| SEQ ID NO: 79 | G to A (bis2 rc) |
| SEQ ID NO: 80 | rc G to A (bis2 rc) |

mPLXND1 Extended Assay 3: 129605381-129606465

| | |
|---|---|
| SEQ ID NO: 81 | genomic reference |
| SEQ ID NO: 82 | C to T (bis1) |
| SEQ ID NO: 83 | rc C to T (bis1) |
| SEQ ID NO: 84 | G to A (bis2 rc) |
| SEQ ID NO: 85 | rc G to A (bis2 rc) |

mPLXND1 Assay 3: 129605881-129605965

| | |
|---|---|
| SEQ ID NO: 86 | genomic reference |
| SEQ ID NO: 87 | C to T (bis1) |
| SEQ ID NO: 88 | rc C to T (bis1) |
| SEQ ID NO: 89 | G to A (bis2 rc) |
| SEQ ID NO: 90 | rc G to A (bis2 rc) |

mRASSF2 Assay + CpG island 20: 4822367-4823486

| | |
|---|---|
| SEQ ID NO: 91 | genomic reference |
| SEQ ID NO: 92 | C to T (bis1) |
| SEQ ID NO: 93 | rc C to T (bis1) |
| SEQ ID NO: 94 | G to A (bis2 rc) |
| SEQ ID NO: 95 | rc G to A (bis2 rc) |

mRASSF2 Extended Assay 20: 4822080-4823182

| | |
|---|---|
| SEQ ID NO: 96 | genomic reference |
| SEQ ID NO: 97 | C to T (bis1) |
| SEQ ID NO: 98 | rc C to T (bis1) |
| SEQ ID NO: 99 | G to A (bis2 rc) |
| SEQ ID NO: 100 | rc G to A (bis2 rc) |

mRASSF2 Assay 20: 4822580-4822682

| | |
|---|---|
| SEQ ID NO: 101 | genomic reference |
| SEQ ID NO: 102 | C to T (bis1) |
| SEQ ID NO: 103 | rc C to T (bis1) |
| SEQ ID NO: 104 | G to A (bis2 rc) |
| SEQ ID NO: 105 | rc G to A (bis2 rc) |
| SEQ ID NO: 106 | mASCL2-F |
| SEQ ID NO: 107 | mLDHB-F |
| SEQ ID NO: 108 | mLGALS3-F |
| SEQ ID NO: 109 | mLOXL3-F |
| SEQ ID NO: 110 | mOSR1-F |
| SEQ ID NO: 111 | mPLXND1-F |
| SEQ ID NO: 112 | mRASSF2-F |
| SEQ ID NO: 113 | mASCL2-R |
| SEQ ID NO: 114 | mLDHB-R |
| SEQ ID NO: 115 | mLGALS3-R |
| SEQ ID NO: 116 | mLOXL3-R |
| SEQ ID NO: 117 | mOSR1-R |
| SEQ ID NO: 118 | mPLXND1-R |
| SEQ ID NO: 119 | mRASSF2-R |

The invention is described by way of the following examples which are to be construed as merely illustrative and not limitative of the scope of the invention.

### Example 1

### Material and Methods

Blood plasma samples from hepatocellular carcinoma (HCC) patients and patients without HCC but with liver cirrhosis (LCi) were collected as defined in the instructions for use (IFU) of the Epi BiSKit (Epigenomics AG). Briefly, for EDTA plasma was prepared by two centrifugation steps. Until processing plasma samples were stored at −70° C.

DNA extraction from plasma samples and bisulfite conversion of DNA was performed with the Epi BiSKit Plasma Kit according to the workflow as defined in the instructions for use (IFU) of the Epi BiSKit (Epigenomics AG).

The PCR was set up with bisulfite DNA yield of an equivalent of about 1 ml plasma in a ready to use multiplex PCR kit (QIAGEN® Multiplex PCR) according to manufactures protocol. PCR oligos (sequences as shown in Table 3) were modified with a 5 ‘phosphate for NGS library preparation. The multiplex PCR profile used a protocol as follows: degeneration at 94° C. for 30 seconds, annealing at 56° C. for 90 seconds, extension step of 30 seconds at 72° C.; 45 cycles.

The PCR product was sequenced paired end with an Illumina MiSeq using a read length of 150 bp.

Fastq files were trimmed to insertions between sequencing adaptors, paired sequences were merged, and sequences filtered for those flanked by primers on both sides reflecting molecules amplified by PCR, called Inserts. Inserts that showed more cytosine than guanine outside of CpG context were turned to their reverse complement to enable assessment of methylation by taking cytosine positions of CpGs into account exclusively. Such inserts were aligned to reference sequences of the assays to assess DNA-methylation: For each assay/sample combination any methylation pattern at CpG sites was assessed by counting occurrence of cytosines and thymidines at CpG positions. Comethylation within a single insert read was defined by cytosine in all CpG positions or all except for one CpG position (allowing an exception one CpG to be different due to any error or SNP at a single CpG site). Quantitative comethylation of a marker in a sample was calculated as number of comethylated insert sequences divided by total number of all inserts found for a sample, normalized by the length of the sequences.

### Results

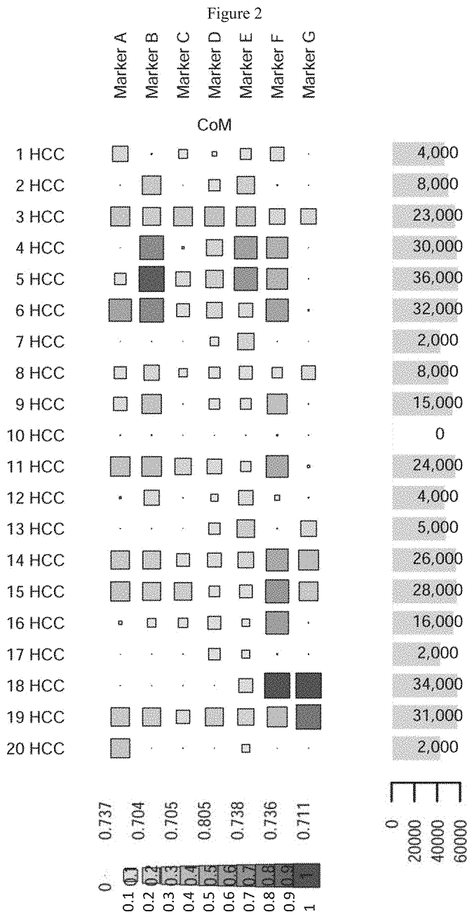

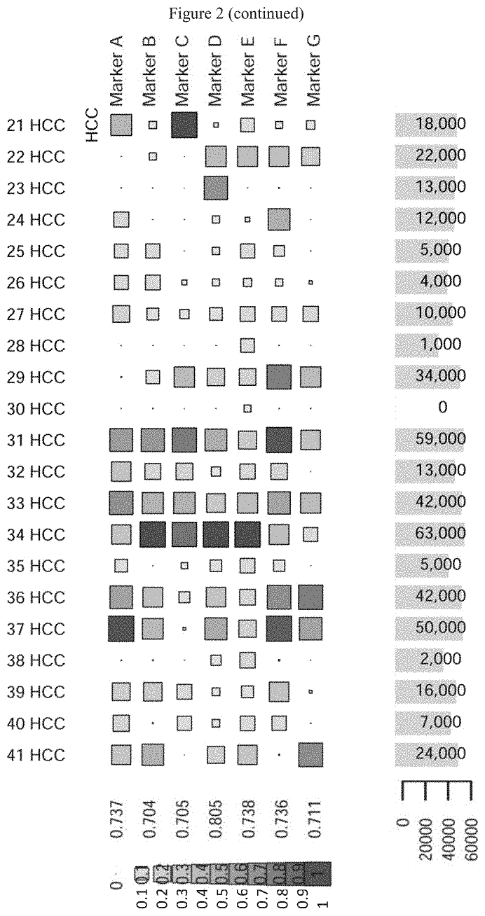

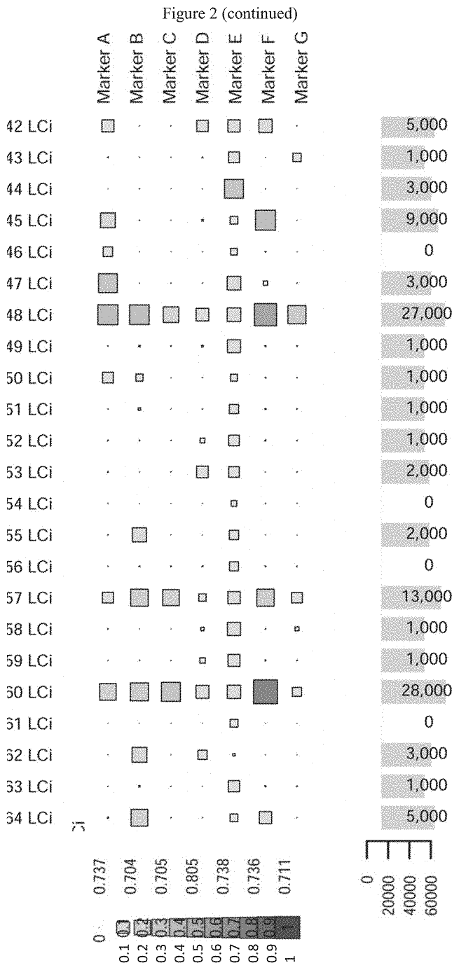

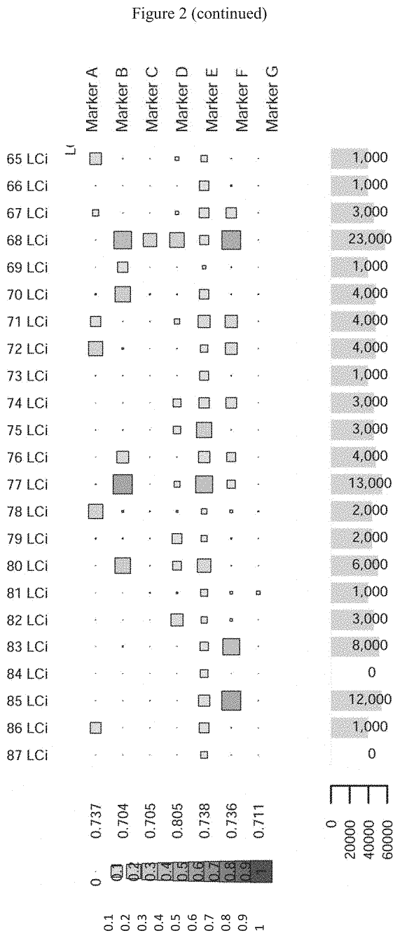

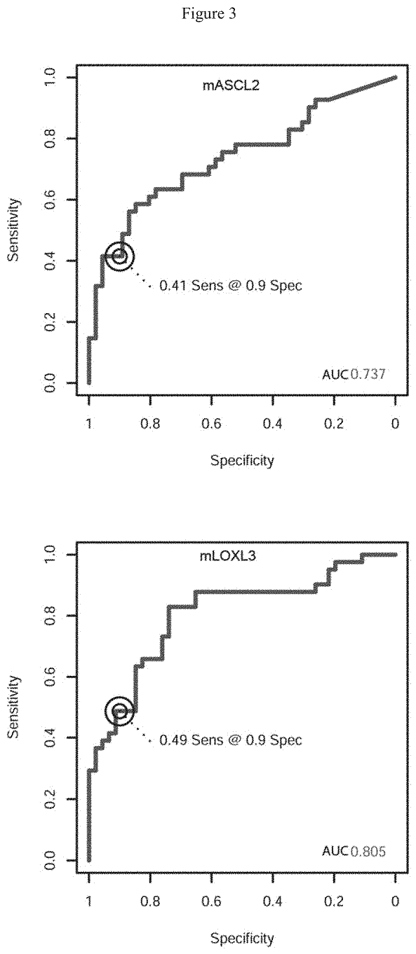

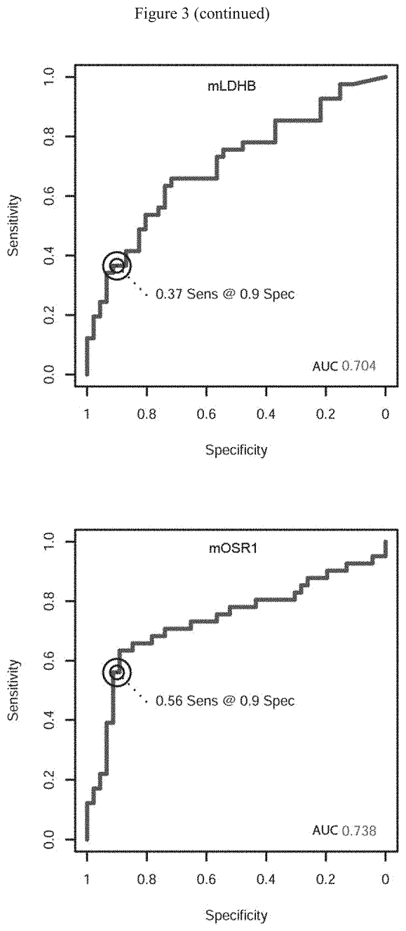

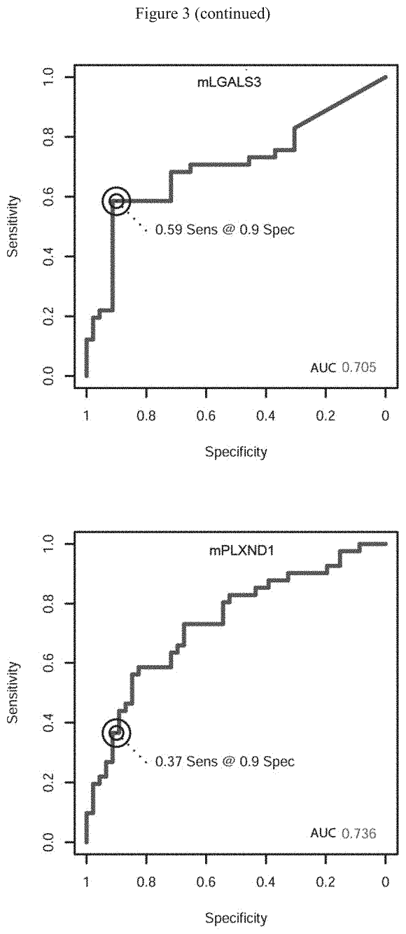

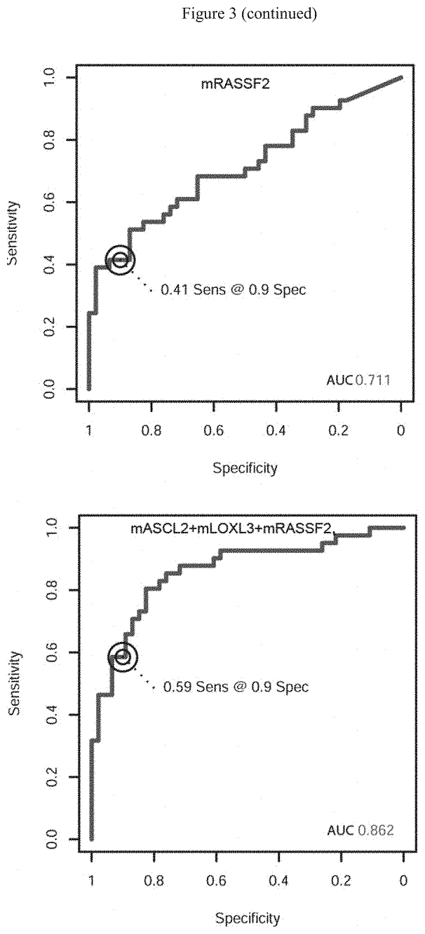

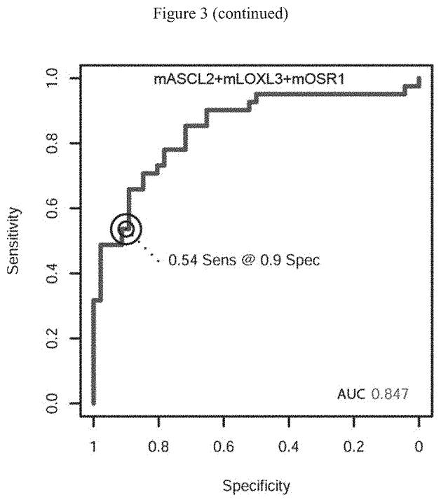

The univariate comparison of DNA-methylation levels found in blood plasma from HCC patients and LCi patients for the set of preselected cancer-markers showed that cancer specific methylation patterns from free circulating tumor cell DNA (ctDNA) can be used to distinguish both groups (summarized in FIG. 2 and in Table 4). The performance as determined by areas under the curves (AUC) of receiver operating characteristic (ROC) was higher than 0.70 for all markers, with good sensitivities at specificity of 90% (FIG. 3). All markers (mASCL2, mLDHB, mLGALS3, mLOXL3, mOSR1, mPLXND1 and mRASSF2show) had methylation patterns with a high grade of comethylation (methylation state of all CpGs within the region assessed is identical in the same molecule), which enables using the amount of reads from molecules with all CpGs methylated to reflect the amount of ctDNA molecules in the template. Within the data set, combination of two or three markers using logistic regression is able to increase the performance, for many combinations above AUC of 0.80 (see FIG. 3 and Tables 1 and 2).

TABLE 4
Data from single marker performance on 41 CRC vs. 46 LCi samples (Sample IDs
by type and number) for different types of data. The values represent the
number of comethylated copies, i.e. means the number of reads found containing
the exact sequence expected from completely methylated molecule.

| ID | mASCL2 | mLDHB | mLGALS3 | mLOXL3 | mOSR1 | mPLXND1 | mRASSF2 |
|---|---|---|---|---|---|---|---|
| 1 HCC | 788 | 29 | 518 | 237 | 582 | 2294 | 3 |
| 2 HCC | 2 | 4228 | 0 | 1270 | 2099 | 35 | 3 |
| 3 HCC | 1633 | 3648 | 3797 | 5990 | 2873 | 3464 | 1868 |
| 4 HCC | 0 | 10935 | 62 | 3341 | 5458 | 9935 | 2 |
| 5 HCC | 364 | 13988 | 1630 | 4293 | 6163 | 9474 | 7 |
| 6 HCC | 2931 | 11086 | 1213 | 3016 | 1044 | 12290 | 30 |
| 7 HCC | 0 | 7 | 0 | 682 | 1730 | 11 | 0 |
| 8 HCC | 403 | 2253 | 467 | 1205 | 886 | 1199 | 1350 |
| 9 HCC | 530 | 4869 | 10 | 1081 | 555 | 7633 | 10 |
| 10 HCC | 6 | 20 | 2 | 5 | 5 | 59 | 9 |
| 11 HCC | 1696 | 5318 | 2395 | 2494 | 496 | 11308 | 63 |
| 12 HCC | 22 | 2184 | 10 | 500 | 1174 | 320 | 18 |
| 13 HCC | 2 | 9 | 0 | 1275 | 2188 | 30 | 1959 |
| 14 HCC | 1376 | 3642 | 1170 | 2117 | 1468 | 11535 | 4406 |
| 15 HCC | 1628 | 3760 | 3057 | 1114 | 843 | 14063 | 3541 |
| 16 HCC | 42 | 556 | 579 | 1770 | 267 | 13062 | 13 |
| 17 HCC | 0 | 4 | 9 | 1334 | 295 | 46 | 9 |
| 18 HCC | 2 | 4 | 6 | 8 | 1057 | 20918 | 11815 |
| 19 HCC | 1419 | 3802 | 1351 | 4672 | 1724 | 7881 | 10131 |
| 20 HCC | 1556 | 0 | 9 | 3 | 286 | 3 | 3 |
| 21 HCC | 2235 | 421 | 12781 | 231 | 970 | 546 | 447 |
| 22 HCC | 2 | 392 | 0 | 7423 | 3406 | 7615 | 2998 |
| 23 HCC | 5 | 9 | 0 | 12596 | 4 | 34 | 6 |
| 24 HCC | 716 | 2 | 3 | 500 | 121 | 10628 | 0 |
| 25 HCC | 578 | 1791 | 2 | 513 | 1104 | 1251 | 5 |
| 26 HCC | 595 | 2015 | 189 | 482 | 313 | 590 | 100 |
| 27 HCC | 1023 | 1117 | 567 | 1636 | 1252 | 2817 | 1808 |
| 28 HCC | 2 | 2 | 0 | 4 | 1015 | 11 | 2 |
| 29 HCC | 8 | 1532 | 5086 | 4171 | 1812 | 16935 | 4728 |
| 30 HCC | 2 | 16 | 2 | 7 | 233 | 16 | 10 |
| 31 HCC | 3132 | 9515 | 10571 | 9454 | 2309 | 20373 | 3994 |
| 32 HCC | 1691 | 2467 | 2554 | 810 | 1354 | 3888 | 2 |
| 33 HCC | 3515 | 6807 | 6229 | 5344 | 3466 | 11913 | 4596 |
| 34 HCC | 1650 | 14535 | 10967 | 18239 | 8996 | 7632 | 1426 |
| 35 HCC | 449 | 4 | 285 | 1347 | 1237 | 1474 | 0 |
| 36 HCC | 3036 | 5169 | 816 | 6215 | 1487 | 15361 | 9679 |
| 37 HCC | 5006 | 6198 | 81 | 9935 | 1985 | 20131 | 6967 |
| 38 HCC | 7 | 24 | 4 | 985 | 1398 | 52 | 6 |
| 39 HCC | 1212 | 4077 | 1774 | 578 | 707 | 7123 | 78 |
| 40 HCC | 926 | 26 | 1558 | 560 | 1167 | 2688 | 12 |
| 41 HCC | 1605 | 7138 | 0 | 3968 | 2913 | 43 | 8762 |
| 42 LiC | 411 | 4 | 5 | 1279 | 752 | 2180 | 5 |
| 43 LiC | 6 | 7 | 3 | 23 | 566 | 0 | 424 |
| 44 LiC | 0 | 6 | 0 | 4 | 2737 | 6 | 0 |
| 45 LiC | 706 | 10 | 0 | 61 | 274 | 7802 | 0 |
| 46 LiC | 241 | 3 | 0 | 3 | 213 | 29 | 0 |
| 47 LiC | 1512 | 2 | 4 | 8 | 976 | 253 | 6 |
| 48 LiC | 1890 | 5134 | 2081 | 1684 | 1006 | 12204 | 3147 |
| 49 LiC | 3 | 46 | 13 | 67 | 857 | 42 | 15 |
| 50 LiC | 314 | 394 | 0 | 11 | 232 | 25 | 6 |
| 51 LiC | 2 | 86 | 2 | 10 | 397 | 38 | 11 |
| 52 LiC | 6 | 18 | 7 | 253 | 537 | 39 | 10 |
| 53 LiC | 7 | 5 | 5 | 1328 | 553 | 5 | 8 |
| 54 LiC | 2 | 2 | 0 | 4 | 166 | 16 | 5 |
| 55 LiC | 4 | 1772 | 0 | 2 | 410 | 4 | 0 |
| 56 LiC | 3 | 21 | 16 | 27 | 387 | 32 | 10 |
| 57 LiC | 341 | 3337 | 2473 | 516 | 789 | 4837 | 726 |
| 58 LiC | 5 | 0 | 0 | 156 | 899 | 23 | 127 |
| 59 LiC | 2 | 10 | 5 | 321 | 714 | 35 | 14 |
| 60 LiC | 987 | 3792 | 3951 | 1779 | 915 | 16128 | 507 |
| 61 LiC | 0 | 0 | 0 | 0 | 287 | 2 | 0 |
| 62 LiC | 0 | 2075 | 0 | 798 | 45 | 3 | 0 |
| 63 LiC | 2 | 40 | 5 | 0 | 603 | 43 | 14 |
| 64 LiC | 11 | 3014 | 8 | 9 | 275 | 1745 | 3 |
| 65 LiC | 529 | 19 | 8 | 216 | 255 | 23 | 6 |
| 66 LiC | 4 | 4 | 2 | 9 | 572 | 73 | 12 |
| 67 LiC | 138 | 7 | 3 | 196 | 611 | 1681 | 8 |
| 68 LiC | 2 | 5783 | 1972 | 3813 | 522 | 10728 | 8 |
| 69 LiC | 0 | 1152 | 0 | 10 | 100 | 34 | 0 |
| 70 LiC | 16 | 3674 | 37 | 37 | 588 | 37 | 19 |
| 71 LiC | 383 | 10 | 11 | 373 | 1069 | 2600 | 9 |
| 72 LiC | 968 | 58 | 0 | 22 | 338 | 2271 | 5 |
| 73 LiC | 6 | 14 | 4 | 12 | 516 | 32 | 2 |
| 74 LiC | 0 | 9 | 0 | 788 | 721 | 1736 | 2 |
| 75 LiC | 4 | 17 | 5 | 711 | 2182 | 20 | 2 |
| 76 LiC | 0 | 1605 | 5 | 14 | 903 | 1208 | 3 |
| 77 LiC | 9 | 8379 | 3 | 457 | 3130 | 971 | 8 |
| 78 LiC | 995 | 74 | 25 | 84 | 189 | 169 | 28 |
| 79 LiC | 11 | 33 | 15 | 1283 | 236 | 62 | 8 |
| 80 LiC | 2 | 3747 | 11 | 969 | 1449 | 23 | 4 |
| 81 LiC | 0 | 0 | 26 | 72 | 306 | 146 | 132 |
| 82 LiC | 3 | 4 | 2 | 2173 | 244 | 142 | 2 |
| 83 LiC | 0 | 39 | 3 | 7 | 447 | 7455 | 8 |
| 84 LiC | 0 | 0 | 0 | 4 | 369 | 0 | 0 |
| 85 LiC | 2 | 4 | 14 | 4 | 855 | 11596 | 10 |
| 86 LiC | 438 | 0 | 6 | 0 | 634 | 30 | 2 |
| 87 LiC | 0 | 3 | 0 | 5 | 280 | 14 | 2 |

### Example 2

### Material and Methods

DNA-methylation data obtained from blood plasma of HCC patients and LCi patients in Example 1 was used to train an algorithm to differentiate the diagnostic groups: Marker candidate performance was characterized by responder operator characteristic (ROC) differentiating HCC vs. LCi for the seven markers. For each of the seven markers a comethylation cutoff was determined at a specificity of 0.9 that was used to determine whether a single marker was classified positive or negative. Marker panel measurements based on the trained thresholds for a sample were defined as number of n positive markers. The 162 samples from sample set 2 were processed, measured and assessed blinded. For each sample each marker was binarized to be either positive or negative using the trained comethylation cutoffs leading to scores of N [0:7] positive markers for each sample. Patient group identity was then de-blinded and performance of the panel described as AUC and Sensitivity at Specificity based on a predefined cutoff of 3+/7. Alpha-fetoprotein (AFP) test results of the patients from which the blood plasma samples were taken were obtained and a combination of methylation with alpha-fetoprotein (AFP) data was assessed using logistic regression.

### Results

The sum of binarized positive call from all seven markers (mASCL2, mLDHB, mLGALS3, mLOXL3, mOSR1, mPLXND1 and mRASSF2) reflecting cancer specific methylation patterns from free circulating tumor cell DNA (ctDNA) could be used to distinguish both groups in the test set based on the trained data. The performance as determined by areas under the curves (AUC) of responder operator characteristic (ROC) was 0.847, with a sensitivity of 0.57 at a specificity of 97% (FIG. 4). A combination with AFP data further increased the discrimination: the performance as determined by areas under the curves (AUC) of responder operator characteristic (ROC) was 0.896, with a sensitivity of 0.68 at a specificity of 97% (FIG. 4). This was unexpected, because AFP is increased in cirrhotic liver tissue, and a combination of AFP with e.g. ultrasound has been found to increase false-positive rates. Also, the worse performing single marker AFP could not have been expected to improven the performance of the methylation marker. In addition the extent of the increase was far larger than could have been expected assuming an additive effect of the methylation markers and AFP.

### Example 3

### Material and Methods

For a subset of four methylation markers (mASCL2, mLGALS3, mLDHB, and mLOXL3), a multiplex Real-time PCR assay was carried out assessing the CpGs of the amplificates with a different method, namely using methylation specific PCR (MSP) and probes according to routine Real-time PCR methods. The assays were measured for the same sample set as in Example 2 using an AB7500 FastDX. Any called Real-time PCR curve was classified as a positive measurement. Measurements for each sample were summed to scores of N [0:4] positive markers for each sample. The performance of the panel is described as AUC. A combination of the n/4 methylation markers with alpha-fetoprotein (AFP) data was assessed using logistic regression and by an OR combination of 2+/4 markers OR 20 ng/mL AFP. In addition, ROCs were calculated for each of the 4 markers based on Cts from Real-time curves and each of four possible combination with AFP were assessed by logistic regression.

### Results

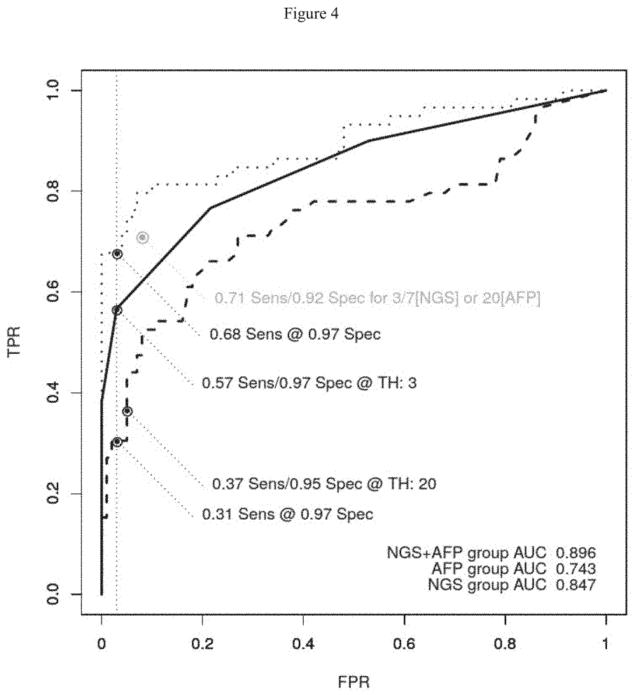

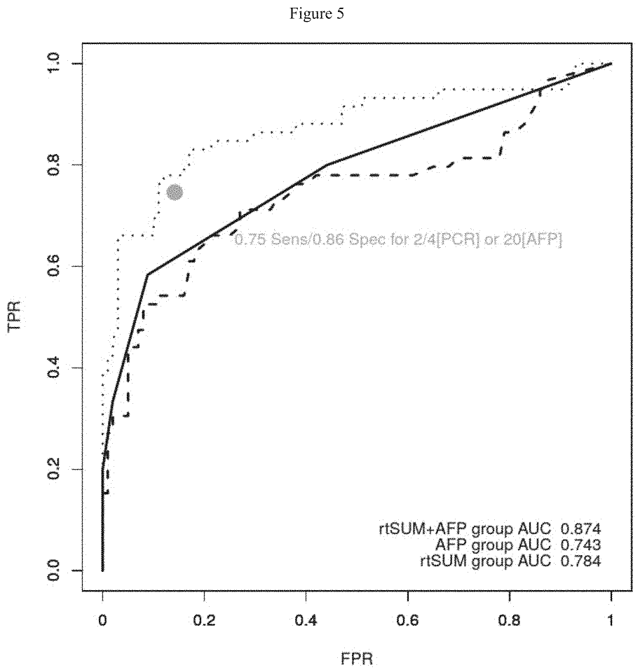

The sum of binarized positive call from all four markers measured with Real-time PCR (mASCL2, mLGALS3, mLDHB, and mLOXL3) reflecting cancer specific methylation patterns from free circulating tumor cell DNA (ctDNA) could be used to distinguish both groups in the test set based on the trained data. The performance as determined by areas under the curves (AUC) of responder operator characteristic (ROC) was 0.784 (FIG. 5). A combination with AFP data further increased the discrimination: the performance as determined by areas under the curves (AUC) of responder operator characteristic (ROC) was 0.874, a combination with fixed cutoffs yielded a Sensitivity of 75% at a Specificity of 86% (FIG. 4).

AUCs based on the four single marker Realtime-PCR Cts and their combination with AFP (AUC of 0.74 for AFP alone) were 0.74 (mASCL2), 0.83 (mASCL2+AFP), 0.69 (mLGALS3), 0.82 (mLGALs3+AFP), 0.65 (mLDHB), 0.83 (mLDHB+AFP), 0.69 (mLOXL3), and 0.85 (mLOXL3+AFP), which was on average a gain of 0.09 in comparison for AFP alone and of 0.14 in comparison to AUCs of single markers alone.

Thus, the unexpected results and the effect of the combination of methylation markers and AFP as seen in Example 2 could technically be confirmed on a smaller subset of markers, on single marker combinations with AFP and with a different technical method.

1. A method of detecting DNA methylation, comprising the step of detecting DNA methylation within at least one genomic DNA polynucleotide selected from the group consisting of polynucleotides having a sequence comprised in SEQ ID NO: 1 (mASCL2), SEQ 5 ID NO: 21 (mLDHB), SEQ ID NO: 36 (mLGALS3), SEQ ID NO: 46 (mLOXL3), SEQ ID NO: 61 (mOSR1), SEQ ID NO: 76 (mPLXND1), or SEQ ID NO: 91 and/or SEQ ID NO: 96 (mRASSF2) in a subject's biological sample comprising genomic DNA, wherein the genomic DNA may comprise DNA derived from liver cancer (LC) cells.

2. The method of claim 1, wherein DNA methylation is detected within at least two, preferably at least three, genomic DNA polynucleotides selected from said group.

3. The method of claim 1 or 2, wherein the method further comprises obtaining the alpha-fetoprotein (AFP) blood level of the subject.

4. The method of any one of claims 1 to 3, comprising the steps of
(a) converting cytosine unmethylated in the 5-position to uracil or another base that does not hybridize to guanine in the genomic DNA of the biological sample; and
(b) detecting DNA methylation within the genomic DNA by detecting unconverted cytosine in the converted DNA of step (a).

5. The method of any one of claims 1 to 4, wherein the detecting of the DNA methylation comprises determining the amount of methylated genomic DNA.

6. The method of any one of claims 1 to 5, wherein the biological sample is a liver tissue sample or a liquid biopsy, preferably a blood sample, a sample comprising cell-free DNA from blood, a blood-derived sample or a saliva sample.

7. The method of any one of claims 1 to 6, wherein the subject has an increased risk of developing LC, is suspected of having LC, has had LC, or has LC.

8. A method for detecting the presence or absence of liver cancer (LC) in a subject, comprising detecting DNA methylation according to any one of claims 1 to 7, wherein the presence of detected methylated genomic DNA indicates the presence of LC and the absence of detected methylated genomic DNA indicates the absence of LC.

9. A method for monitoring a subject having an increased risk of developing liver cancer (LC), suspected of having LC or who has had LC, comprising detecting DNA methylation according to claim 8 repeatedly, wherein the presence of detected methylated genomic DNA indicates the presence of LC and the absence of detected methylated genomic DNA indicates the absence of LC.

10. The method of claim 8 or 9, comprising assessing the AFP blood level of the subject, wherein the presence of detected methylated genomic DNA in combination with an increased AFP blood level indicates the presence of LC and the absence of detected methylated genomic DNA in combination with a normal AFP blood level indicates the absence of LC.

11. An oligonucleotide selected from the group consisting of a primer and probe, comprising a sequence that is substantially identical to a stretch of contiguous nucleotides of one of SEQ ID NOs 2-5 (mASCL2), one of SEQ ID NOs 22-25 (mLDHB), one of SEQ ID NOs 37-40 (mLGALS3), one of SEQ ID NOs 47-50 (mLOXL3), one of SEQ ID NOs 62-65 (mOSR1), one of SEQ ID NOs 77-80 (mPLXND1), one of SEQ ID NOs 92-95 and/or one of SEQ ID NOs 97-100 (mRASSF2), wherein the oligonucleotide preferably is methylation-specific.

12. A kit comprising at least a first and a second oligonucleotide of claim 11.

13. The kit of claim 12, wherein the first and second oligonucleotides are primers forming a primer pair suitable for amplification of DNA having a sequence comprised in one of SEQ ID NOs 2-5 (mASCL2), one of SEQ ID NOs 22-25 (mLDHB), one of SEQ ID NOs 37-40 (mLGALS3), one of SEQ ID NOs 47-50 (mLOXL3), one of SEQ ID NOs 62-65 (mOSR1), one of SEQ ID NOs 77-80 (mPLXND1), one of SEQ ID NOs 92-95 and/or one of SEQ ID NOs 97-100 (mRASSF2).

14. The kit of claim 12 or 13, wherein the kit comprises polynucleotides forming at least two, preferably at least three primer pairs, and wherein each primer pair is suitable for amplification of DNA having a sequence of a different marker mASCL2, mLDHB, mLGALS3, mLOXL3, mOSR1, mPLXND1 and mRASSF2.

15. Use of the method of any one of claims 1 to 7, of the oligonucleotide of claim 10, or of the kit of any one of claims 12 to 14 for the detection of liver cancer (LC), or for monitoring a subject having an increased risk of developing LC, suspected of having LC or that has had LC.
